(19) United States
(12) Patent Application Publication
(10) Pub. No.: US 2017/0290861 A1
(43) Pub. Date: Oct. 12, 2017

(54) TREATMENT OF DIABETIC FOOT ULCER USING PLACENTAL STEM CELLS

(71) Applicant: ANTHROGENESIS CORPORATION, Warren, NJ (US)

(72) Inventors: Steven A. FISCHKOFF, Short Hills, NJ (US); Denesh CHITKARA, East Brunswick, NJ (US); Uri HERZBERG, Bridgewater, NJ (US); Vladimir JANKOVIC, New York, NY (US)

(73) Assignee: Anthrogenesis Corporation, Warren, NJ (US)

(21) Appl. No.: 15/509,710

(22) PCT Filed: Sep. 25, 2015
(86) PCT No.: PCT/US2015/052276

§ 371 Date: Mar. 8, 2017

Related U.S. Application Data
(60) Provisional application No. 62/220,620, filed on Sep. 18, 2015.
(60) Provisional application No. 62/170,757, filed on Jun. 4, 2015.
(60) Provisional application No. 62/151,726, filed on Apr. 23, 2015.
(60) Provisional application No. 62/145,551, filed on Apr. 10, 2015.
(60) Provisional application No. 62/056,008, filed on Sep. 26, 2014.

(51) Int. Cl. A61K 35/50

(52) U.S. Cl. CPC A61K 35/50

(57) ABSTRACT

Provided herein are methods of using tissue culture plastic-adherent placental cells, e.g. placental stem cells, in the treatment of diabetic foot ulcer (DFU).

This application claims priority to U.S. Provisional Patent Application No. 62/056,008, filed Sep. 26, 2014, U.S. Provisional Patent Application No. 62/145,551, filed Apr. 10, 2015, U.S. Provisional Patent Application No. 62/151,726, filed Apr. 23, 2015, U.S. Provisional Patent Application No. 62/170,757, filed Jun. 4, 2015, and U.S. Provisional Patent Application No. 62/220,620, filed Sep. 18, 2015, the disclosures of each of which are incorporated herein by reference in their entireties.

### 1. FIELD

Provided herein are methods of using tissue culture plastic-adherent placental cells, e.g. placental stem cells, in the treatment of diabetic foot ulcer (DFU).

### 2. BACKGROUND

The placenta is a particularly attractive source of stem cells. Because mammalian placentas are plentiful and are normally discarded as medical waste, they represent a unique source of medically-useful stem cells.

### 3. SUMMARY

Provided herein are methods of treating diabetic foot ulcer (DFU) in a subject in need thereof, comprising administering to the subject a therapeutically effective amount of tissue culture plastic-adherent placental cells, e.g., placental stem cells, e.g., CD34⁻, CD10⁺, CD105⁺, CD200⁺ placental stem cells. In a specific embodiment, said placental cells are formulated as a pharmaceutical composition.

In a specific embodiment, a subject with DFU treated in accordance with the methods provided herein has type I diabetes. In another specific embodiment, a subject with DFU treated in accordance with the methods provided herein has type II diabetes. In certain embodiments, a subject treated in accordance with the methods provided herein has more than one DFU, e.g., the subject has more than one DFU on a single foot, or at least one DFU on each foot. In a specific embodiment, the subject has one or more DFU at the bottom of one foot, or both feet.

In certain embodiments, a subject treated in accordance with the methods provided herein has peripheral neuropathy, e.g., damage to one or more of the nerves in the legs and/or feet.

In certain embodiments, a subject treated in accordance with the methods provided herein has DFU with a condition that causes a disruption in the flow of blood in the subject's peripheral vasculature. In a specific embodiment, the subject has peripheral arterial disease (PAD). In certain embodiments, said DFU is caused by and/or associated with PAD. In a specific embodiment, the subject does not have peripheral arterial disease PAD.

In certain embodiments, the methods provided herein result in a detectable improvement of one or more symptoms of DFU in a subject treated in accordance with the methods provided herein. Exemplary symptoms of DFU include, without limitation, sores, ulcers, or blisters on the foot and/or lower leg; pain in the foot (or feet) and/or lower leg; difficulty walking; discoloration in the foot (or feet), e.g., the foot (or feet) appear black, blue, and/or red; and signs of infection (e.g., fever, skin redness, and/or swelling).

In certain embodiments, the methods provided herein comprise administering placental stem cells (e.g., a pharmaceutical composition comprising placental stem cells) to a subject having DFU in an amount and for a time sufficient for detectable improvement in one or more indicia of improvement, wherein said indicia of improvement include (i) reduction in ulcer size; (ii) ulcer closure: skin closure of one or more ulcers without drainage or the need for dressing; (iii) retention of ulcer closure for a specified time period following closure, e.g., 2 weeks, 3 weeks, 4 weeks, 5 weeks, or 6 weeks following closure; (iv) increased time to ulcer closure; (v) improvement in ankle brachial index (ABI), a test that measures blood pressure at the ankle and in the arm while a subject is at rest and then repeated while a subject is in motion (e.g., walking on a treadmill), and which can be used to predict/assess the severity of PAD; (vi) improvement in toe brachial index (TBI), a test analogous to ABI that uses toe blood pressure as opposed to ankle blood pressure; (vii) improvement in transcutaneous oxygen level, i.e., the oxygen level in the tissue beneath the skin close to the ulcer (see, e.g., Ruangsetakit et al., J Wound Care, 2010, 19(5):202-6); (viii) improvement in pulse volume recording, which is a noninvasive vascular test in which blood pressure cuffs and a hand-held ultrasound device are used to obtain information about arterial blood flow in the arms and legs; (ix) time to major amputation, e.g., amputation above the ankle; (x) improvement on the Wagner Grading Scale, which assesses ulcer depth and the presence of osteomyelitis or gangrene using a grading system: grade 0 (pre- or post-ulcerative lesion), grade 1 (partial/full thickness ulcer), grade 2 (probing to tendon or capsule), grade 3 (deep with osteitis), grade 4 (partial foot gangrene), and grade 5 (whole foot gangrene); (xi) improvement in Rutherford criteria, which is used for staging of peripheral arterial disease has seven classification stages: Stage 0—Asymptomatic, Stage 1—mild claudication, Stage 2—moderate claudication, Stage 3—severe claudication, Stage 4—rest pain, Stage 5—ischemic ulceration not exceeding ulcer of the digits of the foot, and Stage 6—severe ischemic ulcers or frank gangrene; and (xii) improvement in leg rest pain score, a 0-10 scale of pain with 0 being pain free and 10 representing maximum pain.

In certain embodiments, the methods provided herein comprise administering placental stem cells (e.g., a pharmaceutical composition comprising placental stem cells) to a subject having DFU in an amount and for a time sufficient for detectable improvement in quality of life of the subject as assessed by, e.g., (i) a 36-item Short Form Health Survey (SF-36) (see, e.g., Ware et al., Medical Care 30(6):473-483); (ii) the Diabetic Foot Ulcer Scale Short Form (DFS-SF), which measures the impact of diabetic foot ulcer on quality of life (see, e.g., Bann et al., Pharmacoeconomics, 2003, 21(17):1277-90); (iii) the Patient Global Impression of Change Scale, to assess changes in neuropathy over time (see, e.g., Kamper et al., J. Man. Manip. Ther., 2009, 17(3):163-170); and/or (iv) the EuroQol5D (EQ-5D™) Scale, which is a health questionnaire used to obtain a descriptive profile and single index value for health status of a patient.

In a specific embodiment of the methods of treatment of DFU described herein, the placental cells (e.g., a pharmaceutical composition comprising placental stem cells) are administered by injection. In another specific embodiment of the methods of treatment of DFU described herein, the placental cells (e.g., a pharmaceutical composition comprising placental stem cells) are administered to a subject being treated by implantation in said subject of a matrix or scaffold comprising placental cells.

In a specific embodiment of the methods of treatment of DFU described herein, the placental cells (e.g., a pharmaceutical composition comprising placental stem cells) are administered intramuscularly. In another specific embodiment of the methods of treatment of DFU described herein, the placental cells (e.g., a pharmaceutical composition comprising placental stem cells) are administered intravenously. In another specific embodiment of the methods of treatment of DFU described herein, the placental cells (e.g., a pharmaceutical composition comprising placental stem cells) are administered subcutaneously. In another specific embodiment of the methods of treatment of DFU described herein, the placental cells (e.g., a pharmaceutical composition comprising placental stem cells) are administered locally. In another specific embodiment of the methods of treatment of DFU described herein, the placental cells (e.g., a pharmaceutical composition comprising placental stem cells) are administered systemically.

In certain embodiments, the methods of treatment of DFU described herein comprise administration of about 1×10³, 3×10³, 5×10³, 1×10⁴, 3×10⁴, 5×10⁴, 1×10⁵, 3×10⁵, 5×10⁵, 1×10⁶, 3×10⁶, 5×10⁶, 1×10⁷, 3×10⁷, 5×10⁷, 1×10⁸, 3×10⁸, 5×10⁸, 1×10⁹, 5×10⁹, or 1×10¹⁰ placental cells (e.g., as part of a pharmaceutical composition comprising placental stem cells). In certain embodiments, the methods of treatment of DFU described herein comprise administration of about 1×10³ to 3×10³, 3×10³ to 5×10³, 5×10³ to 1×10⁴, 1×10⁴ to 3×10⁴, 3×10⁴ to 5×10⁴, 5×10⁴ to 1×10⁵, 1×10⁵ to 3×10⁵, 3×10⁵ to 5×10⁵, 5×10⁵ to 1×10⁶, 1×10⁶ to 3×10⁶, 3×10⁶ to 5×10⁶, 5×10⁶ to 1×10⁷, 1×10⁷ to 3×10⁷, 3×10⁷ to 5×10⁷, 5×10⁷ to 1×10⁸, 1×10⁸ to 3×10⁸, 3×10⁸ to 5×10⁸, 5×10⁸ to 1×10⁹, 1×10⁹ to 5×10⁹, or 5×10⁹ to 1×10¹⁰ placental cells (e.g., as part of a pharmaceutical composition comprising placental stem cells). In a specific embodiment, the methods of treatment of DFU described herein comprise administration of about 3×10⁶ placental cells. In another specific embodiment, the methods of treatment of DFU described herein comprise administration of about 1×10⁷ placental cells. In another specific embodiment, the methods of treatment of DFU described herein comprise administration of about 3×10⁷ placental cells.

In a specific embodiment of the methods of treatment of DFU described herein, the placental stem cells (e.g., a pharmaceutical composition comprising placental stem cells) are administered intramuscularly to a subject more than once, with one week between administrations, e.g., placental cells are administered on day 1 (the first day of administration) and a second dose of placental stem cells (e.g., a pharmaceutical composition comprising placental stem cells) is administered one week later (i.e., on day 8). In another specific embodiment, the methods comprise administration of about 3×10⁶ placental stem cells on each day of administration (i.e., on days 1 and 8). In another specific embodiment, the methods comprise administration of about 1×10⁷ placental cells on each day of administration (i.e., on days 1 and 8). In another specific embodiment, the methods comprise administration of about 3×10⁷ placental cells on each day of administration (i.e., on days 1 and 8). In another specific embodiment, the placental cells are administered are administered to a subject on at least three different occasions, with about one week between administrations. In another specific embodiment, the subject to whom the placental stem cells are administered has PAD.

In another specific embodiment of the methods of treatment of DFU described herein, the placental stem cells (e.g., a pharmaceutical composition comprising placental stem cells) are administered to a subject more than once, with one month between administrations, e.g., placental cells are administered on day 1 (the first day of administration) and a second dose of placental stem cells (e.g., a pharmaceutical composition comprising placental stem cells) is administered about one month later (e.g., on day 27, 28, 29, 30, 31, 32, or 33). In a specific embodiment, the methods comprise administration of about 3×10⁶ placental stem cells on each day of administration (e.g., 3×10⁶ placental stem cells are administered on day 1, and about 3×10⁶ placental stem cells are administered 1 month after day 1, e.g., on day 27, 28, 29, 30, 31, 32, or 33). In another specific embodiment, the methods comprise administration of about 3×10⁷ placental cells on each day of administration (e.g., 3×10⁷ placental stem cells are administered on day 1, and about 3×10⁷ placental stem cells are administered 1 month after day 1, e.g., on day 27, 28, 29, 30, 31, 32, or 33). In another specific embodiment, the placental cells are administered are administered to a subject on at least three different occasions, with about one month between administrations. In another specific embodiment, the subject to whom the placental stem cells are administered has PAD.

In certain embodiments, numbers of circulating endothelial cells of a subject treated in accordance with the methods of treating DFU described herein are determined as a means to assess efficacy of treatment of the subject. Numbers of circulating endothelial cells in a subject treated in accordance with a method provided herein can be determined at any time over the course of the treatment, or before treatment commences. For example, in certain embodiments, numbers of circulating endothelial cells in a subject treated in accordance with a method provided herein are determined (i) before treatment commences (i.e., before placental stem cells are administered to the subject with DFU), e.g., on the day of administration of placental stem cells (but before administration), 1, 2, 3, 4, 5, 6, 7, 8, 9, or 10 days before treatment commences, or 1, 2, 3, 4, or 5 weeks after treatment commences, or 1, 2, 3, 4, 5, or 6 months after treatment commences and (ii) at least once over the course of treatment, e.g., 1, 2, 3, 4, 5, 6, 7, 8, 9, or 10 days after treatment commences, or 1, 2, 3, 4, or 5 weeks after treatment commences, or 1, 2, 3, 4, 5, or 6 months after treatment commences. If a number of circulating endothelial cells determined after treatment commences is less than a number of circulating endothelial cells determined before treatment, then treatment of the subject having DFU can be deemed effective.

In certain embodiments, numbers of circulating endothelial cells in a subject treated in accordance with a method provided herein are determined (i) at a first time point after treatment commences (i.e., after placental stem cells are administered to the subject with DFU), e.g., on the day administration of placental stem cells (but after administration), 1, 2, 3, 4, 5, 6, 7, 8, 9, or 10 days after treatment commences, or 1, 2, 3, 4, or 5 weeks after treatment commences, or 1, 2, 3, 4, 5, or 6 months after treatment commences and (ii) at a second time point after treatment commences, e.g., 1, 2, 3, 4, 5, 6, 7, 8, 9, or 10 days after treatment commences, or 1, 2, 3, 4, or 5 weeks after treatment commences, or 1, 2, 3, 4, 5, or 6 months after treatment commences, wherein the second time point is later in time than the first time point. If the number of circulating endothelial cells determined at the second time point treatment is less than a number of circulating endothelial cells determined at the second time point, then treatment of the subject having DFU can be deemed effective.

In a specific embodiment, provided herein is a method for treating DFU in a subject in need of treatment, wherein the method comprises: (a) determining the number of endothelial cells circulating in the bloodstream of the subject; (b) administering one or more doses of placental stem cells to the subject; and (c) determining the number of endothelial cells circulating in the bloodstream of the subject following the administration of placental stem cells, wherein a decrease in the number of circulating endothelial cells following administration of placental stem cells as compared to the number of circulating endothelial cells before administration of placental stem cells indicates that treatment of DFU in said subject is effective. In certain embodiments, the subject is administered a subsequent dose of a composition comprising CD10⁺, CD34⁻, CD105⁺, CD200⁺ placental stem cells if treatment of DFU in said subject is effective.

In another specific embodiment, provided herein is a method for treating DFU in a subject in need of treatment, wherein the method comprises: (a) administering one or more doses of placental stem cells to the subject; (b) determining the number of endothelial cells circulating in the bloodstream of the subject at a first time point following administration of placental stem cells; and (c) determining the number of endothelial cells circulating in the bloodstream of the subject at a second time point following administration of placental stem cells, wherein a decrease in the number of circulating endothelial cells measured at the second time point as compared to the number of circulating endothelial cells measured at the first time indicates that treatment of DFU in said subject is effective, the subject is administered a subsequent dose of a composition comprising CD10⁺, CD34⁻, CD105⁺, CD200⁺ placental stem cells if treatment of DFU in said subject is effective. In certain embodiments, the subject is administered a subsequent dose of a composition comprising CD10⁺, CD34⁻, CD105⁺, CD200⁺ placental stem cells if treatment of DFU in said subject is effective.

The placental cells used in the methods described herein adhere to tissue culture plastic and are CD34⁻, CD10⁺, CD105⁺ and CD200⁺, as detectable by, e.g., flow cytometry. Further characteristics of the placental cells used in the methods provided herein are described in Section 5.1. Compositions, e.g., pharmaceutical compositions, comprising the placental stem cells to be used in the methods provided herein are described in Section 5.3.

### 3.1 Definitions

As used herein, the term “about,” when referring to a stated numeric value, indicates a value within plus or minus 10% of the stated numeric value.

As used herein, the term “angiogenic,” in reference to the placental derived adherent cells described herein, means that the cells can form vessels or vessel-like sprouts, or that the cells can promote angiogenesis (e.g., the formation of vessels or vessel-like structures) in another population of cells, e.g., endothelial cells.

As used herein, the term “derived” means isolated from or otherwise purified. For example, placental derived adherent cells are isolated from placenta. The term “derived” encompasses cells that are cultured from cells isolated directly from a tissue, e.g., the placenta, and cells cultured or expanded from primary isolates.

As used herein, the term “isolated cell,” e.g., “isolated placental cell,” “isolated placental stem cell,” and the like, means a cell that is substantially separated from other, different cells of the tissue, e.g., placenta, from which the stem cell is derived. A cell is “isolated” if at least 50%, 60%, 70%, 80%, 90%, 95%, or at least 99% of the cells, e.g., non-stem cells, with which the stem cell is naturally associated, or stem cells displaying a different marker profile, are removed from the stem cell, e.g., during collection and/or culture of the stem cell.

As used herein, the term “population of isolated cells” means a population of cells that is substantially separated from other cells of a tissue, e.g., placenta, from which the population of cells is derived.

As used herein, the term “placental cell” refers to a stem cell or progenitor cell that is isolated from a mammalian placenta, e.g., as described in Section 5.1, below, or cultured from cells isolated from a mammalian placenta, either as a primary isolate or a cultured cell, regardless of the number of passages after a primary culture. In certain embodiments, the term “placental cells,” as used herein does not, however, refer to trophoblasts, cytotrophoblasts, syncitiotrophoblasts, angioblasts, hemangioblasts, embryonic germ cells, embryonic stem cells, cells obtained from an inner cell mass of a blastocyst, or cells obtained from a gonadal ridge of a late embryo, e.g., an embryonic germ cell.

As used herein, a placental cell is “positive” for a particular marker when that marker is detectable above background. Detection of a particular marker can, for example, be accomplished either by use of antibodies, or by oligonucleotide probes or primers based on the sequence of the gene or mRNA encoding the marker. For example, a placental cell is positive for, e.g., CD73 because CD73 is detectable on placental cells in an amount detectably greater than background (in comparison to, e.g., an isotype control). A cell is also positive for a marker when that marker can be used to distinguish the cell from at least one other cell type, or can be used to select or isolate the cell when present or expressed by the cell. In the context of, e.g., antibody-mediated detection, “positive,” as an indication a particular cell surface marker is present, means that the marker is detectable using an antibody, e.g., a fluorescently-labeled antibody, specific for that marker; “positive” also refers to a cell exhibiting the marker in an amount that produces a signal, e.g., in a cytometer, that is detectably above background. For example, a cell is “CD200+” where the cell is detectably labeled with an antibody specific to CD200, and the signal from the antibody is detectably higher than that of a control (e.g., background or an isotype control). Conversely, “negative” in the same context means that the cell surface marker is not detectable using an antibody specific for that marker compared a control (e.g., background or an isotype control). For example, a cell is “CD34⁻” where the cell is not reproducibly detectably labeled with an antibody specific to CD34 to a greater degree than a control (e.g., background or an isotype control). Markers not detected, or not detectable, using antibodies are determined to be positive or negative in a similar manner, using an appropriate control. For example, a cell or population of cells can be determined to be OCT-4⁺ if the amount of OCT-4 RNA detected in RNA from the cell or population of cells is detectably greater than background as determined, e.g., by a method of detecting RNA such as RT-PCR, slot blots, etc. Unless otherwise noted herein, cluster of differentiation (“CD”) markers are detected using antibodies. In certain embodiments, OCT-4 is determined to be present, and a cell is “OCT-4⁺” if OCT-4 is detectable using RT-PCR.

As used herein, the terms “subject,” “patient,” and “individual” may be used interchangeably to refer to a mammal being treated with a method of treatment described herein. In a specific embodiment the subject to be treated is a human.

### 4. BRIEF DESCRIPTION OF THE FIGURES

FIG. 1 shows the secretion of selected angiogenic proteins by placental derived adherent cells.

FIG. 2 shows the angiogenic effect of placental derived adherent cells conditioned medium on Human Endothelial Cell (HUVEC) tube formation.

FIG. 3 shows the angiogenic effect of placental derived adherent cells conditioned medium on Human Endothelial Cell migration.

FIG. 4 shows the effect of placental derived adherent cell-conditioned medium on Human Endothelial Cell proliferation.

FIG. 5 shows tube formation of HUVECs and placental derived adherent cells.

FIG. 6 shows the secretion of VEGF and IL-8 by placental derived adherent cells under hypoxic and normoxic conditions.

FIG. 7 shows positive effect of PDAC on angiogenesis in a chick chorioallantois angiogenesis model. bFGF: basic fibroblast growth factor (positive control). MDAMB231: Angiogenic breast cancer cell line (positive control). Y axis: Degree of blood vessel formation.

FIG. 8 shows positive effect of PDAC-conditioned medium (supernatants) on angiogenesis in a chick chorioallantois angiogenesis model. bFGF: basic fibroblast growth factor (positive control). MDAMB231: Angiogenic breast cancer cell line (positive control). Y axis: Degree of blood vessel formation.

FIG. 9: Hydrogen peroxide-generated reactive oxygen species present in cultures of astrocytes, or co-cultures of astrocytes and PDAC. RFU ROS activity: Relative fluorescence units for reactive oxygen species.

FIG. 10 shows increased bloodflow (FIG. 10A) and angiogram score (FIG. 10B) following CD10⁺, CD34⁻, CD105⁺, CD200⁺ placental stem cell administration to mice in a diabetic model of hindlimb ischemia.

FIG. 11 shows an increase in vascular staining using both CD31 and α-smooth muscle actin antibodies following hindlimb ischemia surgery in mice treated with CD10⁺, CD34⁻, CD105⁺, CD200⁺ placental stem cells. Levels of the two markers were analyzed using fluorescent imaging (FIGS. 11A and 11B) and quantified (FIGS. 11C and 11D) as compared to vehicle control-treated animals.

FIG. 12 shows hematoxylin and eosin (H&E) staining of mouse quadriceps muscles following hindlimb ischemia surgery in db/db mice treated with vehicle or two dosages of CD10⁺, CD34⁻, CD105⁺, CD200⁺ placental stem cells.

FIG. 13 shows staining of adipose tissue following hindlimb ischemia and CD10⁺, CD34⁻, CD105⁺, CD200⁺ placental stem cell administration for Arg1 and CD206, two markers of M2 macrophages. Staining was performed 3 days and 14 days following surgery.

FIG. 14 shows cytokine levels in isolated adipocytes with and without LPS stimulation following hindlimb ischemia surgery and CD10⁺, CD34⁻, CD105⁺, CD200⁺ placental stem cell administration.

FIG. 15 shows changes in numbers of circulating endothelial cells from baseline in subjects with healing DFU (15A) and subjects with non-healing DFU (15B) following CD10⁺, CD34⁻, CD105⁺, CD200⁺ placental stem cell administration.

### 5. DETAILED DESCRIPTION

Provided herein are methods of treating diabetic foot ulcer (DFU) in a subject in need thereof, comprising administering to the subject a therapeutically effective amount of tissue culture plastic-adherent placental cells, e.g., placental stem cells, e.g., CD34⁻, CD10⁺, CD105⁺, CD200⁺ placental stem cells. In a specific embodiment, said placental cells are formulated as a pharmaceutical composition.

In a specific embodiment, a subject with DFU treated in accordance with the methods provided herein has type I diabetes. In another specific embodiment, a subject with DFU treated in accordance with the methods provided herein has type II diabetes. In certain embodiments, a subject treated in accordance with the methods provided herein has more than one DFU, i.e., the subject has more than one DFU on a single foot, or at least one DFU on each foot. In a specific embodiment, the subject has one or more DFU at the bottom of one foot, or both feet.

In certain embodiments, a subject treated in accordance with the methods provided herein has peripheral neuropathy, e.g., damage to one or more of the nerves in the legs and/or feet.

In certain embodiments, a subject treated in accordance with the methods provided herein has DFU with a condition that causes a disruption in the flow of blood in the subject's peripheral vasculature. In a specific embodiment, the subject has peripheral arterial disease (PAD). In certain embodiments, said DFU is caused by and/or associated with PAD. In a specific embodiment, the subject does not have peripheral arterial disease (PAD).

In certain embodiments, the methods provided herein result in a detectable improvement of one or more symptoms of DFU in a subject treated in accordance with the methods provided herein. Exemplary symptoms of DFU include, without limitation, sores, ulcers, or blisters on the foot and/or lower leg; pain in the foot (or feet) and/or lower leg; difficulty walking; discoloration in the foot (or feet), e.g., the foot (or feet) appear black, blue, and/or red; and signs of infection (e.g., fever, skin redness, and/or swelling).

In certain embodiments, the methods provided herein comprise administering placental stem cells (e.g., a pharmaceutical composition comprising placental stem cells) to a subject having DFU in an amount and for a time sufficient for detectable improvement in one or more indicia of improvement, wherein said indicia of improvement include (i) reduction in ulcer size; (ii) ulcer closure: skin closure of one or more ulcers without drainage or the need for dressing; (iii) retention of ulcer closure for a specified time period following closure, e.g., 2 weeks, 3 weeks, 4 weeks, 5 weeks, or 6 weeks following closure; (iv) time to ulcer closure; (v) improvement in ankle brachial index (ABI), a test that measures blood pressure at the ankle and in the arm while a subject is at rest and then repeated while a subject is in motion (e.g., walking on a treadmill), and which can be used to predict/assess the severity of PAD; (vi) improvement in toe brachial index (TBI), a test analogous to ABI that uses toe blood pressure as opposed to ankle blood pressure; (vii) improvement in transcutaneous oxygen, i.e., the oxygen level in the tissue beneath the skin close to the ulcer (see, e.g., Ruangsetakit et al., J Wound Care, 2010, 19(5):202-6); (viii) improvement in pulse volume recording, which is a noninvasive vascular test in which blood pressure cuffs and a hand-held ultrasound device are used to obtain information about arterial blood flow in the arms and legs; (ix) time to major amputation, e.g., amputation above the ankle; (x) improvement on the Wagner Grading Scale, which assesses ulcer depth and the presence of osteomyelitis or gangrene using a grading system: grade 0 (pre- or post-ulcerative lesion), grade 1 (partial/full thickness ulcer), grade 2 (probing to tendon or capsule), grade 3 (deep with osteitis), grade 4 (partial foot gangrene), and grade 5 (whole foot gangrene); (xi) improvement in Rutherford criteria, which is used for staging of peripheral arterial disease has seven classification stages: Stage 0 Asymptomatic, Stage 1 mild claudication, Stage 2 moderate claudication, Stage 3 severe claudication, Stage 4 rest pain, Stage 5 ischemic ulceration not exceeding ulcer of the digits of the foot, and Stage 6 severe ischemic ulcers or frank gangrene; and (xii) improvement in leg rest pain score, a 0-10 scale of pain with 0 being pain free and 10 representing maximum pain.

In certain embodiments, the methods provided herein comprise administering placental stem cells (e.g., a pharmaceutical composition comprising placental stem cells) to a subject having DFU in an amount and for a time sufficient for detectable improvement in quality of life of the subject as assessed by, e.g., (i) a 36-item Short Form Health Survey (SF-36) (see, e.g., Ware et al., Medical Care 30(6):473-483); (ii) the Diabetic Foot Ulcer Scale Short Form (DFS-SF), which measures the impact of diabetic foot ulcer on quality of life (see, e.g., Bann et al., Pharmacoeconomics, 2003, 21(17):1277-90); (iii) the Patient Global Impression of Change Scale, to assess changes in neuropathy over time (see, e.g., Kamper et al., J. Man. Manip. Ther., 2009, 17(3):163-170); and/or (iv) the EuroQol5D (EQ-5D™) Scale, which is a health questionnaire used to obtain a descriptive profile and single index value for health status of a patient.

In a specific embodiment of the methods of treatment of DFU described herein, the placental cells (e.g., a pharmaceutical composition comprising placental stem cells) are administered by injection. In another specific embodiment of the methods of treatment of DFU described herein, the placental cells (e.g., a pharmaceutical composition comprising placental stem cells) are administered to a subject being treated by implantation in said subject of a matrix or scaffold comprising placental cells.

In a specific embodiment of the methods of treatment of DFU described herein, the placental cells (e.g., a pharmaceutical composition comprising placental stem cells) are administered intramuscularly. In another specific embodiment of the methods of treatment of DFU described herein, the placental cells (e.g., a pharmaceutical composition comprising placental stem cells) are administered intravenously. In another specific embodiment of the methods of treatment of DFU described herein, the placental cells (e.g., a pharmaceutical composition comprising placental stem cells) are administered subcutaneously. In another specific embodiment of the methods of treatment of DFU described herein, the placental cells (e.g., a pharmaceutical composition comprising placental stem cells) are administered locally. In another specific embodiment of the methods of treatment of DFU described herein, the placental cells (e.g., a pharmaceutical composition comprising placental stem cells) are administered systemically. In certain embodiments, the methods of treatment of DFU described herein comprise administration of about 1×10³, 3×10³, 5×10³, 1×10⁴, 3×10⁴, 5×10⁴, 1×10⁵, 3×10⁵, 5×10⁵, 1×10⁶, 3×10⁶, 5×10⁶, 1×10⁷, 3×10⁷, 5×10⁷, 1×10⁸, 3×10⁸, 5×10⁸, 1×10⁹, 5×10⁹, or 1×10¹⁰ placental cells (e.g., as part of a pharmaceutical composition comprising placental stem cells). In certain embodiments, the methods of treatment of DFU described herein comprise administration of about 1×10³ to 3×10³, 3×10³ to 5×10³, 5×10³ to 1×10⁴, 1×10⁴ to 3×10⁴, 3×10⁴ to 5×10⁴, 5×10⁴ to 1×10⁵, 1×10⁵ to 3×10⁵, 3×10⁵ to 5×10⁵, 5×10⁵ to 1×10⁶, 1×10⁶ to 3×10⁶, 3×10⁶ to 5×10⁶, 5×10⁶ to 1×10⁷, 1×10⁷ to 3×10⁷, 3×10⁷ to 5×10⁷, 5×10⁷ to 1×10⁸, 1×10⁸ to 3×10⁸, 3×10⁸ to 5×10⁸, 5×10⁸ to 1×10⁹, 1×10⁹ to 5×10⁹, or 5×10⁹ to 1×10¹⁰ placental cells (e.g., as part of a pharmaceutical composition comprising placental stem cells). In a specific embodiment, the methods of treatment of DFU described herein comprise administration of about 3×10³ placental cells. In another specific embodiment, the methods of treatment of DFU described herein comprise administration of about 3×10⁴ placental cells. In another specific embodiment, the methods of treatment of DFU described herein comprise administration of about 3×10⁵ placental cells. In another specific embodiment, the methods of treatment of DFU described herein comprise administration of about 3×10⁶ placental cells. In another specific embodiment, the methods of treatment of DFU described herein comprise administration of about 1×10⁷ placental cells. In another specific embodiment, the methods of treatment of DFU described herein comprise administration of about 3×10⁷ placental cells.

In a specific embodiment of the methods of treatment of DFU described herein, the placental stem cells (e.g., a pharmaceutical composition comprising placental stem cells) are administered intramuscularly with one week between administrations, e.g., placental cells are administered on day 1 (the first day of administration) and a second dose of placental stem cells (e.g., a pharmaceutical composition comprising placental stem cells) is administered one week later (i.e., on day 8). In another specific embodiment, the methods comprise administration of about 3×10⁶ placental stem cells on each day of administration (i.e., on days 1 and 8). In another specific embodiment, the methods comprise administration of about 1×10⁷ placental cells on each day of administration (i.e., on days 1 and 8). In another specific embodiment, the methods comprise administration of about 3×10⁷ placental cells on each day of administration (i.e., on days 1 and 8). In another specific embodiment, the subject to whom the placental stem cells are administered has PAD.

The placental cells used in the methods described herein adhere to tissue culture plastic and are CD34⁻, CD10⁺, CD105⁺ and CD200⁺, as detectable by, e.g., flow cytometry. Further characteristics of the placental cells used in the methods provided herein are described in Section 5.1. Compositions, e.g., pharmaceutical compositions, comprising the placental stem cells to be used in the methods provided herein are described in Section 5.3.

### 5.1 Isolated Placental Cells and Isolated Placental Cell Populations

The isolated placental cells, sometimes referred to herein as PDACs, useful in the methods of treatment of DFU provided herein are obtainable from a placenta or part thereof, adhere to a tissue culture substrate and have characteristics of multipotent cells or stem cells, but are not trophoblasts. In certain embodiments, the isolated placental cells useful in the methods disclosed herein have the capacity to differentiate into non-placental cell types.

The isolated placental cells useful in the methods disclosed herein can be either fetal or maternal in origin (that is, can have the genotype of either the fetus or mother, respectively). Preferably, the isolated placental cells and populations of isolated placental cells are fetal in origin. As used herein, the phrase “fetal in origin” or “non-maternal in origin” indicates that the isolated placental cells or populations of isolated placental cells are obtained from the umbilical cord or placental structures associated with the fetus, i.e., that have the fetal genotype. As used herein, the phrase “maternal in origin” indicates that the cells or populations of cells are obtained from a placental structures associated with the mother, e.g., which have the maternal genotype. Isolated placental cells, e.g., PDACs, or populations of cells comprising the isolated placental cells, can comprise isolated placental cells that are solely fetal or maternal in origin, or can comprise a mixed population of isolated placental cells of both fetal and maternal origin. The isolated placental cells, and populations of cells comprising the isolated placental cells, can be identified and selected by the morphological, marker, and culture characteristics discussed below. In certain embodiments, any of the placental cells, e.g., placental stem cells or placental multipotent cells described herein, are autologous to a recipient, e.g., an individual who has a DFU. In certain other embodiments, any of the placental cells, e.g., placental stem cells or placental multipotent cells described herein, are heterologous to a recipient, e.g., an individual who has a DFU.

5.1.1 Physical and Morphological Characteristics

The isolated placental cells described herein (PDACs), when cultured in primary cultures or in cell culture, adhere to the tissue culture substrate, e.g., tissue culture container surface (e.g., tissue culture plastic), or to a tissue culture surface coated with extracellular matrix or ligands such as laminin, collagen (e.g., native or denatured), gelatin, fibronectin, ornithine, vitronectin, and extracellular membrane protein (e.g., MATRIGEL® (BD Discovery Labware, Bedford, Mass.)). The isolated placental cells in culture assume a generally fibroblastoid, stellate appearance, with a number of cytoplasmic processes extending from the central cell body. The cells are, however, morphologically distinguishable from fibroblasts cultured under the same conditions, as the isolated placental cells exhibit a greater number of such processes than do fibroblasts. Morphologically, isolated placental cells are also distinguishable from hematopoietic stem cells, which generally assume a more rounded, or cobblestone, morphology in culture.

In certain embodiments, the isolated placental cells useful in the methods disclosed herein, when cultured in a growth medium, develop embryoid-like bodies. Embryoid-like bodies are noncontiguous clumps of cells that can grow on top of an adherent layer of proliferating isolated placental cells. The term “embryoid-like” is used because the clumps of cells resemble embryoid bodies, clumps of cells that grow from cultures of embryonic stem cells. Growth medium in which embryoid-like bodies can develop in a proliferating culture of isolated placental cells includes medium comprising, e.g., DMEM-LG (e.g., from Gibco); 2% fetal calf serum (e.g., from Hyclone Labs.); 1× insulin-transferrin-selenium (ITS); 1× linoleic acid-bovine serum albumin (LA-BSA); 10⁻⁹ M dexamethasone (e.g., from Sigma); 10⁻⁴ M ascorbic acid 2-phosphate (e.g., from Sigma); epidermal growth factor 10 ng/mL (e.g., from R&D Systems); and platelet-derived growth factor (PDGF-BB) 10 ng/mL (e.g., from R&D Systems).

5.1.2 Cell Surface, Molecular and Genetic Markers

The isolated placental cells, e.g., isolated multipotent placental cells or isolated placental stem cells, and populations of such isolated placental cells, useful in the methods disclosed herein, e.g., the methods of treatment of a DFU of a subject, are tissue culture plastic-adherent human placental cells that have characteristics of multipotent cells or stem cells, and express a plurality of markers that can be used to identify and/or isolate the cells, or populations of cells that comprise the stem cells. In certain embodiments, the PDACs are angiogenic, e.g., in vitro or in vivo. The isolated placental cells, and placental cell populations described herein (that is, two or more isolated placental cells) include placental cells and placental cell-containing cell populations obtained directly from the placenta, or any part thereof (e.g., chorion, placental cotyledons, or the like). Isolated placental cell populations also include populations of (that is, two or more) isolated placental cells in culture, and a population in a container, e.g., a bag. The isolated placental cells described herein are not bone marrow-derived mesenchymal cells, adipose-derived mesenchymal stem cells, or mesenchymal cells obtained from umbilical cord blood, placental blood, or peripheral blood. Placental cells, e.g., placental multipotent cells and placental cells, useful in the methods and compositions described herein are described herein and, e.g., in U.S. Pat. Nos. 7,311,904; 7,311,905; and 7,468,276; and in U.S. Patent Application Publication No. 2007/0275362, the disclosures of which are hereby incorporated by reference in their entireties.

In certain embodiments, the isolated placental cells are isolated placental stem cells. In certain other embodiments, the isolated placental cells are isolated placental multipotent cells. In one embodiment, the isolated placental cells, e.g., PDACs, are CD34⁻, CD10⁺ and CD105⁺ as detected by flow cytometry. In another specific embodiment, the isolated CD34⁻, CD10⁺, CD105⁺ placental cells have the potential to differentiate into cells of a neural phenotype, cells of an osteogenic phenotype, and/or cells of a chondrogenic phenotype. In another specific embodiment, the isolated CD34⁻, CD10⁺, CD105⁺ placental cells are additionally CD200⁺. In another specific embodiment, the isolated CD34⁻, CD10⁺, CD105⁺ placental cells are additionally CD45⁻ or CD90⁺. In another specific embodiment, the isolated CD34⁻, CD10⁺, CD105⁺ placental cells are additionally CD45⁻ and CD90′, as detected by flow cytometry. In another specific embodiment, the isolated CD34⁻, CD10⁺, CD105⁺, CD200⁺ placental cells are additionally CD90⁺ or CD45⁻, as detected by flow cytometry. In another specific embodiment, the isolated CD34⁻, CD10⁺, CD105⁺, CD200⁺ placental cells are additionally CD90⁺ and CD45⁻, as detected by flow cytometry, i.e., the cells are CD34⁻, CD10⁺, CD45⁻, CD90⁺, CD105⁺ and CD200⁺. In another specific embodiment, said CD34⁻, CD10⁺, CD45⁻, CD90⁺, CD105⁺, CD200⁺ cells are additionally CD80⁻ and CD86⁻.

In certain embodiments, said placental cells are CD34⁻, CD10⁺, CD105⁺ and CD200⁺, and one or more of CD38⁻, CD45⁻, CD80⁻, CD86⁻, CD133⁻, HLA-DR,DP,DQ⁻, SSEA3⁻, SSEA4⁻, CD29⁺, CD44⁺, CD73⁺, CD90⁺, CD105⁺, HLA-A,B,C⁺, PDL1⁺, ABC-p⁺, and/or OCT-4⁺, as detected by flow cytometry. In other embodiments, any of the CD34⁻, CD10⁺, CD105⁺ cells described above are additionally one or more of CD29⁺, CD38⁻, CD44⁺, CD54′, SH3⁺ or SH4⁺. In another specific embodiment, the cells are additionally CD44⁺. In another specific embodiment of any of the isolated CD34⁻, CD10⁺, CD105⁺ placental cells above, the cells are additionally one or more of CD117⁻, CD133⁻, KDR⁻ (VEGFR2⁻), HLA-A,B,C⁺, HLA-DP,DQ,DR⁻, or Programmed Death-1 Ligand (PDL1)⁺, or any combination thereof.

In another embodiment, the CD34−, CD10+, CD105+ cells are additionally one or more of CD13+, CD29+, CD33+, CD38−, CD44+, CD45−, CD54+, CD62E−, CD62L−, CD62P−, SH3+ (CD73+), SH4+ (CD73+), CD80−, CD86−, CD90+, SH2+ (CD105+), CD106/VCAM+, CD117−, CD144/VE-cadherinlow, CD184/CXCR4−, CD200+, CD133−, OCT-4+, SSEA3−, SSEA4−, ABC-p+, KDR− (VEGFR2−), HLA-A,B,C+, HLA-DP,DQ,DR−, HLA-G−, or Programmed Death-1 Ligand (PDL1)+, or any combination thereof. In a other embodiment, the CD34−, CD10+, CD105+ cells are additionally CD13+, CD29+, CD33+, CD38−, CD44+, CD45−, CD54/ICAM+, CD62E−, CD62L−, CD62P−, SH3+ (CD73+), SH4+ (CD73+), CD80−, CD86−, CD90+, SH2+ (CD105+), CD106/VCAM+, CD117−, CD144/VE-cadherinlow, CD184/CXCR4−, CD200+, CD133−, OCT-4+, SSEA3−, SSEA4−, ABC-p+, KDR− (VEGFR2−), HLA-A,B,C+, HLA-DP,DQ,DR−, HLA-G−, and Programmed Death-1 Ligand (PDL1)+.

In another specific embodiment, any of the placental cells described herein are additionally ABC-p+, as detected by flow cytometry, or OCT-4+ (POU5F1+), as determined by RT-PCR, wherein ABC-p is a placenta-specific ABC transporter protein (also known as breast cancer resistance protein (BCRP) and as mitoxantrone resistance protein (MXR)), and OCT-4 is the Octamer-4 protein (POU5F1). In another specific embodiment, any of the placental cells described herein are additionally SSEA3− or SSEA4−, as determined by flow cytometry, wherein SSEA3 is Stage Specific Embryonic Antigen 3, and SSEA4 is Stage Specific Embryonic Antigen 4. In another specific embodiment, any of the placental cells described herein are additionally SSEA3− and SSEA4−.

In another specific embodiment, any of the placental cells described herein are additionally one or more of MHC-I+ (e.g., HLA-A,B,C+), MHC-II− (e.g., HLA-DP,DQ,DR−) or HLA-G−. In another specific embodiment, any of the placental cells described herein are additionally one or more of MHC-I+ (e.g., HLA-A,B,C+), MHC-II− (e.g., HLA-DP,DQ,DR−) and HLA-G−.

Also provided herein are populations of the isolated placental cells, or populations of cells, e.g., populations of placental cells, comprising, e.g., that are enriched for, the isolated placental cells, that are useful in the methods and compositions disclosed herein. Preferred populations of cells comprising the isolated placental cells, wherein the populations of cells comprise, e.g., at least 10%, 15%, 20%, 25%, 30%, 35%, 40%, 45%, 50%, 55%, 60%, 65%, 70%, 75%, 80%, 85%, 90%, 95% or 98% isolated CD10+, CD105+ and CD34− placental cells; that is, at least 10%, 15%, 20%, 25%, 30%, 35%, 40%, 45%, 50%, 55%, 60%, 65%, 70%, 75%, 80%, 85%, 90%, 95% or 98% of cells in said population are isolated CD10+, CD105+ and CD34− placental cells. In a specific embodiment, the isolated CD34−, CD10+, CD105+ placental cells are additionally CD200+. In another specific embodiment, the isolated CD34−, CD10+, CD105+, CD200+ placental cells are additionally CD90+ or CD45−, as detected by flow cytometry. In another specific embodiment, the isolated CD34−, CD10+, CD105+, CD200+ placental cells are additionally CD90+ and CD45−, as detected by flow cytometry. In another specific embodiment, any of the isolated CD34−, CD10+, CD105+ placental cells described above are additionally one or more of CD29+, CD38−, CD44+, CD54+, SH3+ or SH4+. In another specific embodiment, the isolated CD34−, CD10+, CD105+ placental cells, or isolated CD34−, CD10+, CD105+, CD200+ placental cells, are additionally CD44+. In a specific embodiment of any of the populations of cells comprising isolated CD34−, CD10+, CD105+ placental cells above, the isolated placental cells are additionally one or more of CD13+, CD29+, CD33+, CD38−, CD44+, CD45−, CD54+, CD62E−, CD62L−, CD62P−, SH3+ (CD73+), SH4+(CD73+), CD80−, CD86−, CD90+, SH2+ (CD105+), CD106NCAM+, CD117−, CD144/VE-cadherinlow, CD184/CXCR4−, CD200+, CD133−, OCT-4+, SSEA3−, SSEA4−, ABC-p+, KDR− (VEGFR2−), HLA-A,B,C+, HLA-DP,DQ,DR−, HLA-G−, or Programmed Death-1 Ligand (PDL1)+, or any combination thereof. In another specific embodiment, the CD34−, CD10+, CD105+ cells are additionally CD13+, CD29+, CD33+, CD38−, CD44+, CD45−, CD54/ICAM+, CD62E−, CD62L−, CD62P−, SH3+ (CD73+), SH4+ (CD73+), CD80−, CD86−, CD90+, SH2+ (CD105+), CD106NCAM+, CD117−, CD144NE-cadherinlow, CD184/CXCR4−, CD200+, CD133−, OCT-4+, SSEA3−, SSEA4−, ABC-p+, KDR− (VEGFR2−), HLA-A,B,C+, HLA-DP,DQ,DR−, HLA-G−, and Programmed Death-1 Ligand (PDL1)+.

In certain embodiments, the isolated placental cells useful in the methods and compositions described herein are one or more, or all, of CD10+, CD29+, CD34−, CD38−, CD44+, CD45−, CD54+, CD90+, SH2+, SH3+, SH4+, SSEA3−, SSEA4−, OCT-4+, and ABC-p+, wherein said isolated placental cells are obtained by physical and/or enzymatic disruption of placental tissue. In a specific embodiment, the isolated placental cells are OCT-4+ and ABC-p+. In another specific embodiment, the isolated placental cells are OCT-4+ and CD34−, wherein said isolated placental cells have at least one of the following characteristics: CD10+, CD29+, CD44+, CD45−, CD54+, CD90+, SH3+, SH4+, SSEA3−, and SSEA4−. In another specific embodiment, the isolated placental cells are OCT-4+, CD34−, CD10+, CD29+, CD44+, CD45−, CD54+, CD90+, SH3+, SH4+, SSEA3−, and SSEA4−. In another embodiment, the isolated placental cells are OCT-4+, CD34−, SSEA3−, and SSEA4−. In another specific embodiment, the isolated placental cells are OCT-4+ and CD34−, and is either SH2+ or SH3+. In another specific embodiment, the isolated placental cells are OCT-4+, CD34−, SH2+, and SH3+. In another specific embodiment, the isolated placental cells are OCT-4+, CD34−, SSEA3−, and SSEA4−, and are either SH2+ or SH3+. In another specific embodiment, the isolated placental cells are OCT-4+ and CD34−, and either SH2+ or SH3+, and is at least one of CD10+, CD29+, CD44+, CD45−, CD54+, CD90+, SSEA3−, or SSEA4−. In another specific embodiment, the isolated placental cells are OCT-4+, CD34−, CD10+, CD29+, CD44+, CD45−, CD54+, CD90+, SSEA3−, and SSEA4−, and either SH2+ or SH3+.

In another embodiment, the isolated placental cells useful in the methods and compositions disclosed herein are SH2+, SH3+, SH4+ and OCT-4+. In another specific embodiment, the isolated placental cells are CD10+, CD29+, CD44+, CD54+, CD90+, CD34−, CD45−, SSEA3−, or SSEA4−. In another embodiment, the isolated placental cells are SH2+, SH3+, SH4+, SSEA3− and SSEA4−. In another specific embodiment, the isolated placental cells are SH2+, SH3+, SH4+, SSEA3− and SSEA4-−, CD10+, CD29+, CD44+, CD54+, CD90+, OCT-4+, CD34− or CD45−.

In another embodiment, the isolated placental cells useful in the methods and compositions disclosed herein are CD10+, CD29+, CD34−, CD44+, CD45−, CD54+, CD90+, SH2+, SH3+, and SH4+; wherein said isolated placental cells are additionally one or more of OCT-4+, SSEA3− or SSEA4−.

In certain embodiments, isolated placental cells useful in the methods and compositions disclosed herein are CD200+ or HLA-G−. In a specific embodiment, the isolated placental cells are CD200+ and HLA-G−. In another specific embodiment, the isolated placental cells are additionally CD73+ and CD105+. In another specific embodiment, the isolated placental cells are additionally CD34−, CD38− or CD45−. In another specific embodiment, the isolated placental cells are additionally CD34−, CD38− and CD45−. In another specific embodiment, said stem cells are CD34−, CD38−, CD45−, CD73+ and CD105+. In another specific embodiment, said isolated CD200+ or HLA-G− placental cells facilitate the formation of embryoid-like bodies in a population of placental cells comprising the isolated placental cells, under conditions that allow the formation of embryoid-like bodies. In another specific embodiment, the isolated placental cells are isolated away from placental cells that are not stem or multipotent cells. In another specific embodiment, said isolated placental cells are isolated away from placental cells that do not display these markers.

In another embodiment, a cell population useful in the methods and compositions described herein is a population of cells comprising, e.g., that is enriched for, CD200+, HLA-G− stem cells. In a specific embodiment, said population is a population of placental cells. In various embodiments, at least about 10%, at least about 20%, at least about 30%, at least about 40%, at least about 50%, or at least about 60% of cells in said cell population are isolated CD200+, HLA-G− placental cells. Preferably, at least about 70% of cells in said cell population are isolated CD200+, HLA-G− placental cells. More preferably, at least about 90%, 95%, or 99% of said cells are isolated CD200+, HLA-G− placental cells. In a specific embodiment of the cell populations, said isolated CD200+, HLA-G− placental cells are also CD73+ and CD105+. In another specific embodiment, said isolated CD200+, HLA-G− placental cells are also CD34−, CD38− or CD45−. In another specific embodiment, said isolated CD200+, HLA-G− placental cells are also CD34−, CD38−, CD45−, CD73+ and CD105+. In another embodiment, said cell population produces one or more embryoid-like bodies when cultured under conditions that allow the formation of embryoid-like bodies. In another specific embodiment, said cell population is isolated away from placental cells that are not stem cells. In another specific embodiment, said isolated CD200+, HLA-G− placental cells are isolated away from placental cells that do not display these markers.

In another embodiment, the isolated placental cells useful in the methods and compositions described herein are CD73+, CD105+, and CD200+. In another specific embodiment, the isolated placental cells are HLA-G−. In another specific embodiment, the isolated placental cells are CD34−, CD38− or CD45−. In another specific embodiment, the isolated placental cells are CD34−, CD38− and CD45−. In another specific embodiment, the isolated placental cells are CD34−, CD38, CD45−, and HLA-G−. In another specific embodiment, the isolated CD73+, CD105+, and CD200+ placental cells facilitate the formation of one or more embryoid-like bodies in a population of placental cells comprising the isolated placental cells, when the population is cultured under conditions that allow the formation of embryoid-like bodies. In another specific embodiment, the isolated placental cells are isolated away from placental cells that are not the isolated placental cells. In another specific embodiment, the isolated placental cells are isolated away from placental cells that do not display these markers.

In another embodiment, a cell population useful in the methods and compositions described herein is a population of cells comprising, e.g., that is enriched for, isolated CD73+, CD105+, CD200+ placental cells. In various embodiments, at least about 10%, at least about 20%, at least about 30%, at least about 40%, at least about 50%, or at least about 60% of cells in said cell population are isolated CD73+, CD105+, CD200+ placental cells. In another embodiment, at least about 70% of said cells in said population of cells are isolated CD73+, CD105+, CD200+ placental cells. In another embodiment, at least about 90%, 95% or 99% of cells in said population of cells are isolated CD73+, CD105+, CD200+ placental cells. In a specific embodiment of said populations, the isolated placental cells are HLA-G−. In another specific embodiment, the isolated placental cells are additionally CD34−, CD38− or CD45−. In another specific embodiment, the isolated placental cells are additionally CD34−, CD38− and CD45−. In another specific embodiment, the isolated placental cells are additionally CD34−, CD38−, CD45−, and HLA-G−. In another specific embodiment, said population of cells produces one or more embryoid-like bodies when cultured under conditions that allow the formation of embryoid-like bodies. In another specific embodiment, said population of placental cells is isolated away from placental cells that are not stem cells. In another specific embodiment, said population of placental cells is isolated away from placental cells that do not display these characteristics.

In certain other embodiments, the isolated placental cells are one or more of CD10+, CD29+, CD34−, CD38−, CD44+, CD45−, CD54+, CD90+, SH2+, SH3+, SH4+, SSEA3−, SSEA4−, OCT-4+, HLA-G− or ABC-p+. In a specific embodiment, the isolated placental cells are CD10+, CD29+, CD34−, CD38−, CD44+, CD45−, CD54+, CD90+, SH2+, SH3+, SH4+, SSEA3−, SSEA4−, and OCT-4+. In another specific embodiment, the isolated placental cells are CD10+, CD29+, CD34−, CD38−, CD45−, CD54+, SH2+, SH3+, and SH4+. In another specific embodiment, the isolated placental cells are CD10+, CD29+, CD34−, CD38−, CD45−, CD54+, SH2+, SH3+, SH4+ and OCT-4+. In another specific embodiment, the isolated placental cells are CD10+, CD29+, CD34−, CD38−, CD44+, CD45−, CD54+, CD90+, HLA-G−, SH2+, SH3+, SH4+. In another specific embodiment, the isolated placental cells are OCT-4+ and ABC-p+. In another specific embodiment, the isolated placental cells are SH2+, SH3+, SH4+ and OCT-4+. In another embodiment, the isolated placental cells are OCT-4+, CD34−, SSEA3−, and SSEA4−. In a specific embodiment, said isolated OCT-4+, CD34−, SSEA3−, and SSEA4− placental cells are additionally CD10+, CD29+, CD34−, CD44+, CD45−, CD54+, CD90+, SH2+, SH3+, and SH4+. In another embodiment, the isolated placental cells are OCT-4+ and CD34−, and either SH3+ or SH4+. In another embodiment, the isolated placental cells are CD34− and either CD10+, CD29+, CD44+, CD54+, CD90+, or OCT-4+.

In another embodiment, the isolated placental cells useful in the methods and compositions described herein are CD200+ and OCT-4+. In a specific embodiment, the isolated placental cells are CD73+ and CD105+. In another specific embodiment, said isolated placental cells are HLA-G−. In another specific embodiment, said isolated CD200+, OCT-4+ placental cells are CD34−, CD38− or CD45−. In another specific embodiment, said isolated CD200+, OCT-4+ placental cells are CD34−, CD38− and CD45−. In another specific embodiment, said isolated CD200+, OCT-4+ placental cells are CD34−, CD38−, CD45−, CD73+, CD105+ and HLA-G−. In another specific embodiment, the isolated CD200+, OCT-4+ placental cells facilitate the production of one or more embryoid-like bodies by a population of placental cells that comprises the isolated cells, when the population is cultured under conditions that allow the formation of embryoid-like bodies. In another specific embodiment, said isolated CD200+, OCT-4+ placental cells are isolated away from placental cells that are not stem cells. In another specific embodiment, said isolated CD200+, OCT-4+ placental cells are isolated away from placental cells that do not display these characteristics.

In another embodiment, a cell population useful in the methods and compositions described herein is a population of cells comprising, e.g., that is enriched for, CD200+, OCT-4+ placental cells. In various embodiments, at least about 10%, at least about 20%, at least about 30%, at least about 40%, at least about 50%, or at least about 60% of cells in said cell population are isolated CD200+, OCT-4+ placental cells. In another embodiment, at least about 70% of said cells are said isolated CD200+, OCT-4+ placental cells. In another embodiment, at least about 80%, 90%, 95%, or 99% of cells in said cell population are said isolated CD200+, OCT-4+ placental cells. In a specific embodiment of the isolated populations, said isolated CD200+, OCT-4+ placental cells are additionally CD73+ and CD105+. In another specific embodiment, said isolated CD200+, OCT-4+ placental cells are additionally HLA-G−. In another specific embodiment, said isolated CD200+, OCT-4+ placental cells are additionally CD34−, CD38− and CD45−. In another specific embodiment, said isolated CD200+, OCT-4+ placental cells are additionally CD34−, CD38−, CD45−, CD73+, CD105+ and HLA-G−. In another specific embodiment, the cell population produces one or more embryoid-like bodies when cultured under conditions that allow the formation of embryoid-like bodies. In another specific embodiment, said cell population is isolated away from placental cells that are not isolated CD200+, OCT-4+ placental cells. In another specific embodiment, said cell population is isolated away from placental cells that do not display these markers.

In another embodiment, the isolated placental cells useful in the methods and compositions described herein are CD73+, CD105+ and HLA-G−. In another specific embodiment, the isolated CD73+, CD105+ and HLA-G− placental cells are additionally CD34−, CD38− or CD45−. In another specific embodiment, the isolated CD73+, CD105+, HLA-G− placental cells are additionally CD34−, CD38− and CD45−. In another specific embodiment, the isolated CD73+, CD105+, HLA-G− placental cells are additionally OCT-4+. In another specific embodiment, the isolated CD73+, CD105+, HLA-G− placental cells are additionally CD200+. In another specific embodiment, the isolated CD73+, CD105+, HLA-G− placental cells are additionally CD34−, CD38−, CD45−, OCT-4+ and CD200+. In another specific embodiment, the isolated CD73+, CD105+, HLA-G− placental cells facilitate the formation of embryoid-like bodies in a population of placental cells comprising said cells, when the population is cultured under conditions that allow the formation of embryoid-like bodies. In another specific embodiment, said the isolated CD73+, CD105+, HLA-G− placental cells are isolated away from placental cells that are not the isolated CD73+, CD105+, HLA-G− placental cells. In another specific embodiment, said the isolated CD73+, CD105+, HLA-G− placental cells are isolated away from placental cells that do not display these markers.

In another embodiment, a cell population useful in the methods and compositions described herein is a population of cells comprising, e.g., that is enriched for, isolated CD73+, CD105+ and HLA-G− placental cells. In various embodiments, at least about 10%, at least about 20%, at least about 30%, at least about 40%, at least about 50%, or at least about 60% of cells in said population of cells are isolated CD73+, CD105+, HLA-G− placental cells. In another embodiment, at least about 70% of cells in said population of cells are isolated CD73+, CD105+, HLA-G− placental cells. In another embodiment, at least about 90%, 95% or 99% of cells in said population of cells are isolated CD73+, CD105+, HLA-G− placental cells. In a specific embodiment of the above populations, said isolated CD73+, CD105+, HLA-G− placental cells are additionally CD34−, CD38− or CD45−. In another specific embodiment, said isolated CD73+, CD105+, HLA-G− placental cells are additionally CD34−, CD38− and CD45−. In another specific embodiment, said isolated CD73+, CD105+, HLA-G− placental cells are additionally OCT-4+. In another specific embodiment, said isolated CD73+, CD105+, HLA-G− placental cells are additionally CD200+. In another specific embodiment, said isolated CD73+, CD105+, HLA-G− placental cells are additionally CD34−, CD38−, CD45−, OCT-4+ and CD200+. In another specific embodiment, said cell population is isolated away from placental cells that are not CD73+, CD105+, HLA-G− placental cells. In another specific embodiment, said cell population is isolated away from placental cells that do not display these markers.

In another embodiment, the isolated placental cells useful in the methods and compositions described herein are CD73+ and CD105+ and facilitate the formation of one or more embryoid-like bodies in a population of isolated placental cells comprising said CD73+, CD105+ cells when said population is cultured under conditions that allow formation of embryoid-like bodies. In another specific embodiment, said isolated CD73+, CD105+ placental cells are additionally CD34−, CD38− or CD45−. In another specific embodiment, said isolated CD73+, CD105+ placental cells are additionally CD34−, CD38− and CD45−. In another specific embodiment, said isolated CD73+, CD105+ placental cells are additionally OCT-4+. In another specific embodiment, said isolated CD73+, CD105+ placental cells are additionally OCT-4+, CD34−, CD38− and CD45−. In another specific embodiment, said isolated CD73+, CD105+ placental cells are isolated away from placental cells that are not said cells. In another specific embodiment, said isolated CD73+, CD105+ placental cells are isolated away from placental cells that do not display these characteristics.

In another embodiment, a cell population useful in the methods and compositions described herein is a population of cells comprising, e.g., that is enriched for, isolated placental cells that are CD73+, CD105+ and facilitate the formation of one or more embryoid-like bodies in a population of isolated placental cells comprising said cells when said population is cultured under conditions that allow formation of embryoid-like bodies. In various embodiments, at least about 10%, at least about 20%, at least about 30%, at least about 40%, at least about 50%, or at least about 60% of cells in said population of cells are said isolated CD73+, CD105+ placental cells. In another embodiment, at least about 70% of cells in said population of cells are said isolated CD73+, CD105+ placental cells. In another embodiment, at least about 90%, 95% or 99% of cells in said population of cells are said isolated CD73+, CD105+ placental cells. In a specific embodiment of the above populations, said isolated CD73+, CD105+ placental cells are additionally CD34−, CD38− or CD45−. In another specific embodiment, said isolated CD73+, CD105+ placental cells are additionally CD34−, CD38− and CD45−. In another specific embodiment, said isolated CD73+, CD105+ placental cells are additionally OCT-4+. In another specific embodiment, said isolated CD73+, CD105+ placental cells are additionally CD200+. In another specific embodiment, said isolated CD73+, CD105+ placental cells are additionally CD34−, CD38, CD45−, OCT-4+ and CD200+. In another specific embodiment, said cell population is isolated away from placental cells that are not said isolated CD73+, CD105+ placental cells. In another specific embodiment, said cell population is isolated away from placental cells that do not display these markers.

In another embodiment, the isolated placental cells useful in the methods and compositions described herein are OCT-4+ and facilitate formation of one or more embryoid-like bodies in a population of isolated placental cells comprising said cells when cultured under conditions that allow formation of embryoid-like bodies. In a specific embodiment, said isolated OCT-4+ placental cells are additionally CD73+ and CD105+. In another specific embodiment, said isolated OCT-4+ placental cells are additionally CD34−, CD38, or CD45−. In another specific embodiment, said isolated OCT-4+ placental cells are additionally CD200+. In another specific embodiment, said isolated OCT-4+ placental cells are additionally CD73+, CD105+, CD200+, CD34−, CD38, and CD45−. In another specific embodiment, said isolated OCT-4+ placental cells are isolated away from placental cells that are not OCT-4+ placental cells. In another specific embodiment, said isolated OCT-4+ placental cells are isolated away from placental cells that do not display these characteristics.

In another embodiment, a cell population useful in the methods and compositions described herein is a population of cells comprising, e.g., that is enriched for, isolated placental cells that are OCT-4+ and facilitate the formation of one or more embryoid-like bodies in a population of isolated placental cells comprising said cells when said population is cultured under conditions that allow formation of embryoid-like bodies. In various embodiments, at least about 10%, at least about 20%, at least about 30%, at least about 40%, at least about 50%, or at least about 60% of cells in said population of cells are said isolated OCT-4+ placental cells. In another embodiment, at least about 70% of cells in said population of cells are said isolated OCT-4+ placental cells. In another embodiment, at least about 80%, 90%, 95% or 99% of cells in said population of cells are said isolated OCT-4+ placental cells. In a specific embodiment of the above populations, said isolated OCT-4+ placental cells are additionally CD34−, CD38− or CD45−. In another specific embodiment, said isolated OCT-4+ placental cells are additionally CD34−, CD38− and CD45−. In another specific embodiment, said isolated OCT-4+ placental cells are additionally CD73+ and CD105+. In another specific embodiment, said isolated OCT-4+ placental cells are additionally CD200+. In another specific embodiment, said isolated OCT-4+ placental cells are additionally CD73+, CD105+, CD200+, CD34−, CD38−, and CD45−. In another specific embodiment, said cell population is isolated away from placental cells that are not said cells. In another specific embodiment, said cell population is isolated away from placental cells that do not display these markers.

In another embodiment, the isolated placental cells useful in the methods and compositions described herein are isolated HLA-A,B,C+, CD45−, CD133− and CD34− placental cells. In another embodiment, a cell population useful in the methods and compositions described herein is a population of cells comprising isolated placental cells, wherein at least about 70%, at least about 80%, at least about 90%, at least about 95% or at least about 99% of cells in said isolated population of cells are isolated HLA-A,B,C+, CD45−, CD133− and CD34− placental cells. In a specific embodiment, said isolated placental cell or population of isolated placental cells is isolated away from placental cells that are not HLA-A,B,C+, CD45−, CD133− and CD34− placental cells. In another specific embodiment, said isolated placental cells are non-maternal in origin. In another specific embodiment, said isolated population of placental cells are substantially free of maternal components; e.g., at least about 40%, 45%, 5-0%, 55%, 60%, 65%, 70%, 75%, 90%, 85%, 90%, 95%, 98% or 99% of said cells in said isolated population of placental cells are non-maternal in origin.

In another embodiment, the isolated placental cells useful in the methods and compositions described herein are isolated CD10+, CD13+, CD33+, CD45−, CD117− and CD133− placental cells. In another embodiment, a cell population useful in the methods and compositions described herein is a population of cells comprising isolated placental cells, wherein at least about 70%, at least about 80%, at least about 90%, at least about 95% or at least about 99% of cells in said population of cells are isolated CD10+, CD13+, CD33+, CD45−, CD117− and CD133− placental cells. In a specific embodiment, said isolated placental cells or population of isolated placental cells is isolated away from placental cells that are not said isolated placental cells. In another specific embodiment, said isolated CD10+, CD13+, CD33+, CD45−, CD117− and CD133− placental cells are non-maternal in origin, i.e., have the fetal genotype. In another specific embodiment, at least about 40%, 45%, 50%, 55%, 60%, 65%, 70%, 75%, 90%, 85%, 90%, 95%, 98% or 99% of said cells in said isolated population of placental cells, are non-maternal in origin. In another specific embodiment, said isolated placental cells or population of isolated placental cells are isolated away from placental cells that do not display these characteristics.

In another embodiment, the isolated placental cells useful in the methods and compositions described herein are isolated CD10−, CD33−, CD44+, CD45−, and CD117− placental cells. In another embodiment, a cell population useful for the in the methods and compositions described herein is a population of cells comprising, e.g., enriched for, isolated placental cells, wherein at least about 70%, at least about 80%, at least about 90%, at least about 95% or at least about 99% of cells in said population of cells are isolated CD10−, CD33−, CD44+, CD45−, and CD117− placental cells. In a specific embodiment, said isolated placental cell or population of isolated placental cells is isolated away from placental cells that are not said cells. In another specific embodiment, said isolated placental cells are non-maternal in origin. In another specific embodiment, at least about 40%, 45%, 50%, 55%, 60%, 65%, 70%, 75%, 90%, 85%, 90%, 95%, 98% or 99% of said cells in said cell population are non-maternal in origin. In another specific embodiment, said isolated placental cell or population of isolated placental cells is isolated away from placental cells that do not display these markers.

In another embodiment, the isolated placental cells useful in the methods and compositions described herein are isolated CD10−, CD13−, CD33−, CD45−, and CD117− placental cells. In another embodiment, a cell population useful for in the methods and compositions described herein is a population of cells comprising, e.g., enriched for, isolated CD10−, CD13−, CD33−, CD45−, and CD117− placental cells, wherein at least about 70%, at least about 80%, at least about 90%, at least about 95% or at least about 99% of cells in said population are CD10−, CD13−, CD33−, CD45−, and CD117− placental cells. In a specific embodiment, said isolated placental cells or population of isolated placental cells are isolated away from placental cells that are not said cells. In another specific embodiment, said isolated placental cells are non-maternal in origin. In another specific embodiment, at least about 40%, 45%, 50%, 55%, 60%, 65%, 70%, 75%, 90%, 85%, 90%, 95%, 98% or 99% of said cells in said cell population are non-maternal in origin. In another specific embodiment, said isolated placental cells or population of isolated placental cells is isolated away from placental cells that do not display these characteristics.

In another embodiment, the isolated placental cells useful in the methods and compositions described herein are HLA A,B,C+, CD45−, CD34−, and CD133−, and are additionally CD10+, CD13+, CD38+, CD44+, CD90+, CD105+, CD200+ and/or HLA-G−, and/or negative for CD117. In another embodiment, a cell population useful in the methods described herein is a population of cells comprising isolated placental cells, wherein at least about 20%, 25%, 30%, 35%, 40%, 45%, 50%, 55%, 60%, 65%, 70%, 75%, 80%, 85%, 90%, 95%, 98% or about 99% of the cells in said population are isolated placental cells that are HLA A,B,C−, CD45−, CD34−, CD133−, and that are additionally positive for CD10, CD13, CD38, CD44, CD90, CD105, CD200, and/or negative for CD117 and/or HLA-G−. In a specific embodiment, said isolated placental cells or population of isolated placental cells are isolated away from placental cells that are not said cells. In another specific embodiment, said isolated placental cells are non-maternal in origin. In another specific embodiment, at least about 40%, 45%, 50%, 55%, 60%, 65%, 70%, 75%, 90%, 85%, 90%, 95%, 98% or 99% of said cells in said cell population are non-maternal in origin. In another specific embodiment, said isolated placental cells or population of isolated placental cells are isolated away from placental cells that do not display these markers.

In another embodiment, the isolated placental cells useful in the methods and compositions described herein are isolated placental cells that are CD200+ and CD10+, as determined by antibody binding, and CD117−, as determined by both antibody binding and RT-PCR. In another embodiment, the isolated placental cells useful in the methods and compositions described herein are isolated placental cells, e.g., placental stem cells or placental multipotent cells, that are CD10+, CD29−, CD54+, CD200+, HLA-G−, MHC class I+ and β-2-microglobulin+. In another embodiment, isolated placental cells useful in the methods and compositions described herein are placental cells wherein the expression of at least one cellular marker is at least two-fold higher than for a mesenchymal stem cell (e.g., a bone marrow-derived mesenchymal stem cell). In another specific embodiment, said isolated placental cells are non-maternal in origin. In another specific embodiment, at least about 40%, 45%, 50%, 55%, 60%, 65%, 70%, 75%, 90%, 85%, 90%, 95%, 98% or 99% of said cells in said cell population are non-maternal in origin.

In another embodiment, the isolated placental cells useful in the methods and compositions described herein are isolated placental cells, e.g., placental stem cells or placental multipotent cells, that are one or more of CD10+, CD29+, CD44+, CD45−, CD54/ICAM+, CD62E−, CD62L−, CD62P−, CD80−, CD86−, CD103−, CD104−, CD105+, CD106NCAM+, CD144/VE-cadherinlow, CD184/CXCR4−, β2-microglobulinlow, MHC-Ilow, MHC-II−, HLA-Glow, and/or PDL1low. In a specific embodiment, the isolated placental cells are at least CD29+ and CD54+. In another specific embodiment, the isolated placental cells are at least CD44+ and CD106+. In another specific embodiment, the isolated placental cells are at least CD29+.

In another embodiment, a cell population useful in the methods and compositions described herein comprises isolated placental cells, and at least 50%, 60%, 70%, 80%, 90%, 95%, 98% or 99% of the cells in said cell population are isolated placental cells that are one or more of CD10+, CD29+, CD44+, CD45−, CD54/ICAM+, CD62-E−, CD62-L−, CD62-P−, CD80−, CD86−, CD103−, CD104−, CD105+, CD106NCAM+, CD144/VE-cadherindim, CD184/CXCR4−, β2-microglobulindim, HLA-Idim, HLA-II−, HLA-Gdim, and/or PDL1dim. In another specific embodiment, at least 50%, 60%, 70%, 80%, 90%, 95%, 98% or 99% of cells in said cell population are CD10+, CD29+, CD44+, CD45−, CD54/ICAM+, CD62-E−, CD62-L−, CD62-P−, CD80−, CD86−, CD103−, CD104−, CD105+, CD106NCAM+, CD144/VE-cadherindim, CD184/CXCR4−, β2-microglobulindim, MHC-Idim, MHC-II−, HLA-Gdim, and PDL1dim.

In another embodiment, the isolated placental cells useful in the methods and compositions described herein are isolated placental cells that are one or more, or all, of CD10+, CD29+, CD34−, CD38−, CD44+, CD45−, CD54+, CD90+, SH2+, SH3+, SH4+, SSEA3−, SSEA4−, OCT-4+, and ABC-p+, where ABC-p is a placenta-specific ABC transporter protein (also known as breast cancer resistance protein (BCRP) and as mitoxantrone resistance protein (MXR)), wherein said isolated placental cells are obtained by perfusion of a mammalian, e.g., human, placenta that has been drained of cord blood and perfused to remove residual blood.

In another specific embodiment of any of the above characteristics, expression of the cellular marker (e.g., cluster of differentiation or immunogenic marker) is determined by flow cytometry; in another specific embodiment, expression of the marker is determined by RT-PCR.

Gene profiling confirms that isolated placental cells, and populations of isolated placental cells, are distinguishable from other cells, e.g., mesenchymal stem cells, e.g., bone marrow-derived mesenchymal stem cells. The isolated placental cells described herein can be distinguished from, e.g., mesenchymal stem cells on the basis of the expression of one or more genes, the expression of which is significantly higher in the isolated placental cells, or in certain isolated umbilical cord stem cells, in comparison to bone marrow-derived mesenchymal stem cells. In particular, the isolated placental cells, useful in the methods of treatment provided herein, can be distinguished from mesenchymal stem cells on the basis of the expression of one or more genes, the expression of which is significantly higher (that is, at least twofold higher) in the isolated placental cells than in an equivalent number of bone marrow-derived mesenchymal stem cells, wherein the one or more genes are ACTG2, ADARB1, AMIGO2, ARTS-1, B4GALT6, BCHE, C11orf9, CD200, COL4A1, COL4A2, CPA4, DMD, DSC3, DSG2, ELOVL2, F2RL1, FLJ10781, GATA6, GPR126, GPRC5B, HLA-G, ICAM1, IER3, IGFBP7, IL1A, IL6, IL18, KRT18, KRT8, LIPG, LRAP, MATN2, MEST, NFE2L3, NUAK1, PCDH7, PDLIM3, PKP2, RTN1, SERPINB9, ST3GAL6, ST6GALNAC5, SLC12A8, TCF21, TGFB2, VTN, ZC3H12A, or a combination of any of the foregoing, when the cells are grown under equivalent conditions. See, e.g., U.S. Patent Application Publication No. 2007/0275362, the disclosure of which is incorporated herein by reference in its entirety. In certain specific embodiments, said expression of said one ore more genes is determined, e.g., by RT-PCR or microarray analysis, e.g., using a U133-A microarray (Affymetrix). In another specific embodiment, said isolated placental cells express said one or more genes when cultured for a number of population doublings, e.g., anywhere from about 3 to about 35 population doublings, in a medium comprising DMEM-LG (e.g., from Gibco); 2% fetal calf serum (e.g., from Hyclone Labs.); 1× insulin-transferrin-selenium (ITS); 1× linoleic acid-bovine serum albumin (LA-BSA); 10-9 M dexamethasone (e.g., from Sigma); 10-4 M ascorbic acid 2-phosphate (e.g., from Sigma); epidermal growth factor 10 ng/mL (e.g., from R&D Systems); and platelet-derived growth factor (PDGF-BB) 10 ng/mL (e.g., from R&D Systems). In another specific embodiment, the isolated placental cell-specific or isolated umbilical cord cell-specific gene is CD200.

Specific sequences for these genes can be found in GenBank at accession nos. NM_001615 (ACTG2), BC065545 (ADARB1), (NM_181847 (AMIGO2), AY358590 (ARTS-1), BC074884 (B4GALT6), BC008396 (BCHE), BCO20196 (Cllorf9), BC031103 (CD200), NM_001845 (COL4A1), NM_001846 (COL4A2), BC052289 (CPA4), BC094758 (DMD), AF293359 (DSC3), NM_001943 (DSG2), AF338241 (ELOVL2), AY336105 (F2RL1), NM_018215 (FLJ10781), AY416799 (GATA6), BC075798 (GPR126), NM_016235 (GPRCSB), AF340038 (ICAM1), BC000844 (IER3), BC066339 (IGFBP7), BC013142 (IL1A), BT019749 (IL6), BC007461 (IL18), (BC072017) KRT18, BC075839 (KRT8), BC060825 (LIPG), BC065240 (LRAP), BC010444 (MATN2), BC011908 (MEST), BC068455 (NFE2L3), NM_014840 (NUAK1), AB006755 (PCDH7), NM_014476 (PDLIM3), BC126199 (PKP-2), BC090862 (RTN1), BC002538 (SERPINB9), BCO23312 (ST3GAL6), BC001201 (ST6GALNAC5), BC126160 or BC065328 (SLC12A8), BC025697 (TCF21), BC096235 (TGFB2), BC005046 (VTN), and BC005001 (ZC3H12A) as of March 2008.

In certain specific embodiments, said isolated placental cells express each of ACTG2, ADARB1, AMIGO2, ARTS-1, B4GALT6, BCHE, C11orf9, CD200, COL4A1, COL4A2, CPA4, DMD, DSC3, DSG2, ELOVL2, F2RL1, FLJ10781, GATA6, GPR126, GPRCSB, HLA-G, ICAM1, IER3, IGFBP7, IL1A, IL6, IL18, KRT18, KRT8, LIPG, LRAP, MATN2, MEST, NFE2L3, NUAK1, PCDH7, PDLIM3, PKP2, RTN1, SERPINB9, ST3GAL6, ST6GALNAC5, SLC12A8, TCF21, TGFB2, VTN, and ZC3H12A at a detectably higher level than an equivalent number of bone marrow-derived mesenchymal stem cells, when the cells are grown under equivalent conditions.

In specific embodiments, the placental cells express CD200 and ARTS1 (aminopeptidase regulator of type 1 tumor necrosis factor); ARTS-1 and LRAP (leukocyte-derived arginine aminopeptidase); IL6 (interleukin-6) and TGFB2 (transforming growth factor, beta 2); IL6 and KRT18 (keratin 18); IER3 (immediate early response 3), MEST (mesoderm specific transcript homolog) and TGFB2; CD200 and IER3; CD200 and IL6; CD200 and KRT18; CD200 and LRAP; CD200 and MEST; CD200 and NFE2L3 (nuclear factor (erythroid-derived 2)-like 3); or CD200 and TGFB2 at a detectably higher level than an equivalent number of bone marrow-derived mesenchymal stem cells (BM-MSCs) wherein said bone marrow-derived mesenchymal stem cells have undergone a number of passages in culture equivalent to the number of passages said isolated placental cells have undergone. In other specific embodiments, the placental cells express ARTS-1, CD200, IL6 and LRAP; ARTS-1, IL6, TGFB2, IER3, KRT18 and MEST; CD200, IER3, IL6, KRT18, LRAP, MEST, NFE2L3, and TGFB2; ARTS-1, CD200, IER3, IL6, KRT18, LRAP, MEST, NFE2L3, and TGFB2; or IER3, MEST and TGFB2 at a detectably higher level than an equivalent number of bone marrow-derived mesenchymal stem cells BM-MSCs, wherein said bone marrow-derived mesenchymal stem cells have undergone a number of passages in culture equivalent to the number of passages said isolated placental cells have undergone.

Expression of the above-referenced genes can be assessed by standard techniques. For example, probes based on the sequence of the gene(s) can be individually selected and constructed by conventional techniques. Expression of the genes can be assessed, e.g., on a microarray comprising probes to one or more of the genes, e.g., an Affymetrix GENECHIP® Human Genome U133A 2.0 array, or an Affymetrix GENECHIP® Human Genome U133 Plus 2.0 (Santa Clara, Calif.). Expression of these genes can be assessed even if the sequence for a particular GenBank accession number is amended because probes specific for the amended sequence can readily be generated using well-known standard techniques.

The level of expression of these genes can be used to confirm the identity of a population of isolated placental cells, to identify a population of cells as comprising at least a plurality of isolated placental cells, or the like. Populations of isolated placental cells, the identity of which is confirmed, can be clonal, e.g., populations of isolated placental cells expanded from a single isolated placental cell, or a mixed population of stem cells, e.g., a population of cells comprising solely isolated placental cells that are expanded from multiple isolated placental cells, or a population of cells comprising isolated placental cells, as described herein, and at least one other type of cell.

The level of expression of these genes can be used to select populations of isolated placental cells. For example, a population of cells, e.g., clonally-expanded cells, may be selected if the expression of one or more of the genes listed above is significantly higher in a sample from the population of cells than in an equivalent population of mesenchymal stem cells. Such selecting can be of a population from a plurality of isolated placental cell populations, from a plurality of cell populations, the identity of which is not known, etc.

Isolated placental cells can be selected on the basis of the level of expression of one or more such genes as compared to the level of expression in said one or more genes in, e.g., a mesenchymal stem cell control, for example, the level of expression in said one or more genes in an equivalent number of bone marrow-derived mesenchymal stem cells. In one embodiment, the level of expression of said one or more genes in a sample comprising an equivalent number of mesenchymal stem cells is used as a control. In another embodiment, the control, for isolated placental cells tested under certain conditions, is a numeric value representing the level of expression of said one or more genes in mesenchymal stem cells under said conditions.

In certain embodiments, the placental cells (e.g., PDACs) useful in the methods provided herein, do not express CD34−, as detected by immunolocalization, after exposure to 1 to 100 ng/mL VEGF for 4 to 21 days. In a specific embodiment, said placental adherent cells are adherent to tissue culture plastic. In another specific embodiment, said population of cells induce endothelial cells to form sprouts or tube-like structures when cultured in the presence of an angiogenic factor such as vascular endothelial growth factor (VEGF), epithelial growth factor (EGF), platelet derived growth factor (PDGF) or basic fibroblast growth factor (bFGF), e.g., on a substrate such as MATRIGEL™.

In another aspect, the PDACs provided herein, a population of cells, e.g., a population of PDACs, or a population of cells wherein at least about 50%, 60%, 70%, 80%, 90%, 95% or 98% of cells in said isolated population of cells are PDACs, secrete one or more, or all, of VEGF, HGF, IL-8, MCP-3, FGF2, follistatin, G-CSF, EGF, ENA-78, GRO, IL-6, MCP-1, PDGF-BB, TIMP-2, uPAR, or galectin-1, e.g., into culture medium in which the cell, or cells, are grown. In another embodiment, the PDACs express increased levels of CD202b, IL-8 and/or VEGF under hypoxic conditions (e.g., less than about 5% O2) compared to normoxic conditions (e.g., about 20% or about 21% O2).

In another embodiment, any of the PDACS or populations of cells comprising PDACs described herein can cause the formation of sprouts or tube-like structures in a population of endothelial cells in contact with said placental derived adherent cells. In a specific embodiment, the PDACs are co-cultured with human endothelial cells, which form sprouts or tube-like structures, or support the formation of endothelial cell sprouts, e.g., when cultured in the presence of extracellular matrix proteins such as collagen type I and IV, and/or angiogenic factors such as vascular endothelial growth factor (VEGF), epithelial growth factor (EGF), platelet derived growth factor (PDGF) or basic fibroblast growth factor (bFGF), e.g., in or on a substrate such as placental collagen or MATRIGEL™ for at least 4 days. In another embodiment, any of the populations of cells comprising placental derived adherent cells, described herein, secrete angiogenic factors such as vascular endothelial growth factor (VEGF), hepatocyte growth factor (HGF), platelet derived growth factor (PDGF), basic fibroblast growth factor (bFGF), or Interleukin-8 (IL-8) and thereby can induce human endothelial cells to form sprouts or tube-like structures when cultured in the presence of extracellular matrix proteins such as collagen type I and IV e.g., in or on a substrate such as placental collagen or MATRIGEL™.

In another embodiment, any of the above populations of cells comprising placental derived adherent cells (PDACs) secretes angiogenic factors. In specific embodiments, the population of cells secretes vascular endothelial growth factor (VEGF), hepatocyte growth factor (HGF), platelet derived growth factor (PDGF), basic fibroblast growth factor (bFGF), and/or interleukin-8 (IL-8). In other specific embodiments, the population of cells comprising PDACs secretes one or more angiogenic factors and thereby induces human endothelial cells to migrate in an in vitro wound healing assay. In other specific embodiments, the population of cells comprising placental derived adherent cells induces maturation, differentiation or proliferation of human endothelial cells, endothelial progenitors, myocytes or myoblasts.

The isolated placental cells described herein display the above characteristics (e.g., combinations of cell surface markers and/or gene expression profiles) in primary culture, or during proliferation in medium comprising, e.g., DMEM-LG (Gibco), 2% fetal calf serum (FCS) (Hyclone Laboratories), 1× insulin-transferrin-selenium (ITS), 1× lenolenic-acid-bovine-serum-albumin (LA-BSA), 10-9 M dexamethasone (Sigma), 10-4M ascorbic acid 2-phosphate (Sigma), epidermal growth factor (EGF)10 ng/ml (R&D Systems), platelet derived-growth factor (PDGF-BB) 10 ng/ml (R&D Systems), and 100 U penicillin/1000 U streptomycin.

In certain embodiments of any of the placental cells disclosed herein, the cells are human. In certain embodiments of any of the placental cells disclosed herein, the cellular marker characteristics or gene expression characteristics are human markers or human genes.

In another specific embodiment of said isolated placental cells or populations of cells comprising the isolated placental cells, said cells or population have been expanded, for example, passaged at least, about, or no more than, 1, 2, 3, 4, 5, 6, 7, 8, 9, 10, 11, 12, 13, 14, 15, 16, 17, 18, 19, or 20 times, or more, or proliferated for at least, about, or no more than, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 population doublings. In another specific embodiment of said isolated placental cells or populations of cells comprising the isolated placental cells, said cells or population are primary isolates. In another specific embodiment of the isolated placental cells, or populations of cells comprising isolated placental cells, that are disclosed herein, said isolated placental cells are fetal in origin (that is, have the fetal genotype).

In certain embodiments, said isolated placental cells do not differentiate during culturing in growth medium, i.e., medium formulated to promote proliferation, e.g., during proliferation in growth medium. In another specific embodiment, said isolated placental cells do not require a feeder layer in order to proliferate. In another specific embodiment, said isolated placental cells do not differentiate in culture in the absence of a feeder layer, solely because of the lack of a feeder cell layer.

In another embodiment, cells useful in the methods and compositions described herein are isolated placental cells, wherein a plurality of said isolated placental cells are positive for aldehyde dehydrogenase (ALDH), as assessed by an aldehyde dehydrogenase activity assay. Such assays are known in the art (see, e.g., Bostian and Betts, Biochem. J., 173, 787, (1978)). In a specific embodiment, said ALDH assay uses Aldefluor® (Aldagen, Inc., Ashland, Oreg.) as a marker of aldehyde dehydrogenase activity. In a specific embodiment, said plurality is between about 3% and about 25% of cells in said population of cells. In another embodiment, provided herein is a population of isolated umbilical cord cells, e.g., multipotent isolated umbilical cord cells, wherein a plurality of said isolated umbilical cord cells are positive for aldehyde dehydrogenase, as assessed by an aldehyde dehydrogenase activity assay that uses Aldefluor® as an indicator of aldehyde dehydrogenase activity. In a specific embodiment, said plurality is between about 3% and about 25% of cells in said population of cells. In another embodiment, said population of isolated placental cells or isolated umbilical cord cells shows at least three-fold, or at least five-fold, higher ALDH activity than a population of bone marrow-derived mesenchymal stem cells having about the same number of cells and cultured under the same conditions.

In certain embodiments of any of the populations of cells comprising the isolated placental cells described herein, the placental cells in said populations of cells are substantially free of cells having a maternal genotype; e.g., at least 40%, 45%, 50%, 55%, 60%, 65%, 70%, 75%, 80%, 85%, 90%, 95%, 98% or 99% of the placental cells in said population have a fetal genotype. In certain other embodiments of any of the populations of cells comprising the isolated placental cells described herein, the populations of cells comprising said placental cells are substantially free of cells having a maternal genotype; e.g., at least 40%, 45%, 50%, 55%, 60%, 65%, 70%, 75%, 80%, 85%, 90%, 95%, 98% or 99% of the cells in said population have a fetal genotype.

In a specific embodiment of any of the above isolated placental cells or cell populations of isolated placental cells, the karyotype of the cells, or at least about 95% or about 99% of the cells in said population, is normal. In another specific embodiment of any of the above placental cells or cell populations, the cells, or cells in the population of cells, are non-maternal in origin.

Isolated placental cells, or populations of isolated placental cells, bearing any of the above combinations of markers, can be combined in any ratio. Any two or more of the above isolated placental cell populations can be combined to form an isolated placental cell population. For example, an population of isolated placental cells can comprise a first population of isolated placental cells defined by one of the marker combinations described above, and a second population of isolated placental cells defined by another of the marker combinations described above, wherein said first and second populations are combined in a ratio of about 1:99, 2:98, 3:97, 4:96, 5:95, 10:90, 20:80, 30:70, 40:60, 50:50, 60:40, 70:30, 80:20, 90:10, 95:5, 96:4, 97:3, 98:2, or about 99:1. In like fashion, any three, four, five or more of the above-described isolated placental cells or isolated placental cells populations can be combined.

Isolated placental cells useful in the methods and compositions described herein can be obtained, e.g., by disruption of placental tissue, with or without enzymatic digestion (see Section 5.2.3) or perfusion (see Section 5.2.4). For example, populations of isolated placental cells can be produced according to a method comprising perfusing a mammalian placenta that has been drained of cord blood and perfused to remove residual blood; perfusing said placenta with a perfusion solution; and collecting said perfusion solution, wherein said perfusion solution after perfusion comprises a population of placental cells that comprises isolated placental cells; and isolating a plurality of said isolated placental cells from said population of cells. In a specific embodiment, the perfusion solution is passed through both the umbilical vein and umbilical arteries and collected after it exudes from the placenta. In another specific embodiment, the perfusion solution is passed through the umbilical vein and collected from the umbilical arteries, or passed through the umbilical arteries and collected from the umbilical vein.

In various embodiments, the isolated placental cells, contained within a population of cells obtained from perfusion of a placenta, are at least 50%, 60%, 70%, 80%, 90%, 95%, 99% or at least 99.5% of said population of placental cells. In another specific embodiment, the isolated placental cells collected by perfusion comprise fetal and maternal cells. In another specific embodiment, the isolated placental cells collected by perfusion are at least 50%, 60%, 70%, 80%, 90%, 95%, 99% or at least 99.5% fetal cells.

In another specific embodiment, provided herein is a composition comprising a population of the isolated placental cells, as described herein, collected by perfusion, wherein said composition comprises at least a portion of the perfusion solution used to collect the isolated placental cells.

Isolated populations of the isolated placental cells described herein can be produced by digesting placental tissue with a tissue-disrupting enzyme to obtain a population of placental cells comprising the cells, and isolating, or substantially isolating, a plurality of the placental cells from the remainder of said placental cells. The whole, or any part of, the placenta can be digested to obtain the isolated placental cells described herein. In specific embodiments, for example, said placental tissue can be a whole placenta, an amniotic membrane, chorion, a combination of amnion and chorion, or a combination of any of the foregoing. In other specific embodiment, the tissue-disrupting enzyme is trypsin or collagenase. In various embodiments, the isolated placental cells, contained within a population of cells obtained from digesting a placenta, are at least 50%, 60%, 70%, 80%, 90%, 95%, 99% or at least 99.5% of said population of placental cells.

The isolated populations of placental cells described above, and populations of isolated placental cells generally, can comprise about, at least, or no more than 1×10³, 3×10³, 5×10³, 1×10⁴, 3×10⁴, 5×10⁴, 1×10⁵, 3×10⁵, 5×10⁵, 1×10⁶, 3×10⁶, 5×10⁶, 1×10⁷, 3×10⁷, 5×10⁷, 1×10⁸, 3×10⁸, 5×10⁸, 1×10⁹, 5×10⁹, or 1×10¹⁰ isolated placental cells (e.g., as part of a pharmaceutical composition comprising placental stem cells) or between about 1×10³ to 3×10³, 3×10³ to 5×10³, 5×10³ to 1×10⁴, 1×10⁴ to 3×10⁴, 3×10⁴ to 5×10⁴, 5×10⁴ to 1×10⁵, 1×10⁵ to 3×10⁵, 3×10⁵ to 5×10⁵, 5×10⁵ to 1×10⁶, 1×10⁶ to 3×10⁶, 3×10⁶ to 5×10⁶, 5×10⁶ to 1×10⁷, 1×10⁷ to 3×10⁷, 3×10⁷ to 5×10⁷, 5×10⁷ to 1×10⁸, 1×10⁸ to 3×10⁸, 3×10⁸ to 5×10⁸, 5×10⁸ to 1×10⁹, 1×10⁹ to 5×10⁹, or 5×10⁹ to 1×10¹⁰ isolated placental cells (e.g., as part of a pharmaceutical composition comprising placental stem cells). Populations of isolated placental cells useful in the methods of treatment described herein comprise at least 50%, 55%, 60%, 65%, 70%, 75%, 80%, 85%, 90%, 95%, 98%, or 99% viable isolated placental cells, e.g., as determined by, e.g., trypan blue exclusion.

### 5.2 Methods of Obtaining Isolated Placental Cells

5.2.1 Stem Cell Collection Composition

Further provided herein are methods of collecting and isolating placental cells, e.g., the isolated placental cells described in Section 5.1, above. Generally, such cells are obtained from a mammalian placenta using a physiologically-acceptable solution, e.g., a cell collection composition. An exemplary cell collection composition is described in detail in related U.S. Patent Application Publication No. 2007/0190042, entitled “Improved Medium for Collecting Placental Stem Cells and Preserving Organs,” the disclosure of which is incorporated herein by reference in its entirety

The cell collection composition can comprise any physiologically-acceptable solution suitable for the collection and/or culture of cells, e.g., the isolated placental cells described herein, for example, a saline solution (e.g., phosphate-buffered saline, Kreb's solution, modified Kreb's solution, Eagle's solution, 0.9% NaCl. etc.), a culture medium (e.g., DMEM, H.DMEM, etc.), and the like.

The cell collection composition can comprise one or more components that tend to preserve isolated placental cells, that is, prevent the isolated placental cells from dying, or delay the death of the isolated placental cells, reduce the number of isolated placental cells in a population of cells that die, or the like, from the time of collection to the time of culturing. Such components can be, e.g., an apoptosis inhibitor (e.g., a caspase inhibitor or JNK inhibitor); a vasodilator (e.g., magnesium sulfate, an antihypertensive drug, atrial natriuretic peptide (ANP), adrenocorticotropin, corticotropin-releasing hormone, sodium nitroprusside, hydralazine, adenosine triphosphate, adenosine, indomethacin or magnesium sulfate, a phosphodiesterase inhibitor, etc.); a necrosis inhibitor (e.g., 2-(1H-Indol-3-yl)-3-pentylamino-maleimide, pyrrolidine dithiocarbamate, or clonazepam); a TNF-α inhibitor; and/or an oxygen-carrying perfluorocarbon (e.g., perfluorooctyl bromide, perfluorodecyl bromide, etc.).

The cell collection composition can comprise one or more tissue-degrading enzymes, e.g., a metalloprotease, a serine protease, a neutral protease, an RNase, or a DNase, or the like. Such enzymes include, but are not limited to, collagenases (e.g., collagenase I, II, III or IV, a collagenase from Clostridium histolyticum, etc.); dispase, thermolysin, elastase, trypsin, LIBERASE, hyaluronidase, and the like.

The cell collection composition can comprise a bacteriocidally or bacteriostatically effective amount of an antibiotic. In certain non-limiting embodiments, the antibiotic is a macrolide (e.g., tobramycin), a cephalosporin (e.g., cephalexin, cephradine, cefuroxime, cefprozil, cefaclor, cefixime or cefadroxil), a clarithromycin, an erythromycin, a penicillin (e.g., penicillin V) or a quinolone (e.g., ofloxacin, ciprofloxacin or norfloxacin), a tetracycline, a streptomycin, etc. In a particular embodiment, the antibiotic is active against Gram(+) and/or Gram(−) bacteria, e.g., Pseudomonas aeruginosa, Staphylococcus aureus, and the like. In one embodiment, the antibiotic is gentamycin, e.g., about 0.005% to about 0.01% (w/v) in culture medium

The cell collection composition can also comprise one or more of the following compounds: adenosine (about 1 mM to about 50 mM); D-glucose (about 20 mM to about 100 mM); magnesium ions (about 1 mM to about 50 mM); a macromolecule of molecular weight greater than 20,000 daltons, in one embodiment, present in an amount sufficient to maintain endothelial integrity and cellular viability (e.g., a synthetic or naturally occurring colloid, a polysaccharide such as dextran or a polyethylene glycol present at about 25 g/l to about 100 g/l, or about 40 g/l to about 60 g/l); an antioxidant (e.g., butylated hydroxyanisole, butylated hydroxytoluene, glutathione, vitamin C or vitamin E present at about 25 μM to about 100 μM); a reducing agent (e.g., N-acetylcysteine present at about 0.1 mM to about 5 mM); an agent that prevents calcium entry into cells (e.g., verapamil present at about 2 μM to about 25 μM); nitroglycerin (e.g., about 0.05 g/L to about 0.2 g/L); an anticoagulant, in one embodiment, present in an amount sufficient to help prevent clotting of residual blood (e.g., heparin or hirudin present at a concentration of about 1000 units/1 to about 100,000 units/1); or an amiloride containing compound (e.g., amiloride, ethyl isopropyl amiloride, hexamethylene amiloride, dimethyl amiloride or isobutyl amiloride present at about 1.0 μM to about 5 μM).

5.2.2 Collection and Handling of Placenta

Generally, a human placenta is recovered shortly after its expulsion after birth. In a preferred embodiment, the placenta is recovered from a patient after informed consent and after a complete medical history of the patient is taken and is associated with the placenta. Preferably, the medical history continues after delivery. Such a medical history can be used to coordinate subsequent use of the placenta or the isolated placental cells harvested therefrom. For example, isolated human placental cells can be used, in light of the medical history, for personalized medicine for the infant associated with the placenta, or for parents, siblings or other relatives of the infant.

Prior to recovery of isolated placental cells, the umbilical cord blood and placental blood are preferably removed. In certain embodiments, after delivery, the cord blood in the placenta is recovered. The placenta can be subjected to a conventional cord blood recovery process. Typically a needle or cannula is used, with the aid of gravity, to exsanguinate the placenta (see, e.g., Anderson, U.S. Pat. No. 5,372,581; Hessel et al., U.S. Pat. No. 5,415,665). The needle or cannula is usually placed in the umbilical vein and the placenta can be gently massaged to aid in draining cord blood from the placenta. Such cord blood recovery may be performed commercially, e.g., LifeBank USA, Cedar Knolls, N.J. Preferably, the placenta is gravity drained without further manipulation so as to minimize tissue disruption during cord blood recovery.

Typically, a placenta is transported from the delivery or birthing room to another location, e.g., a laboratory, for recovery of cord blood and collection of stem cells by, e.g., perfusion or tissue dissociation. The placenta is preferably transported in a sterile, thermally insulated transport device (maintaining the temperature of the placenta between 20-28° C.), for example, by placing the placenta, with clamped proximal umbilical cord, in a sterile zip-lock plastic bag, which is then placed in an insulated container. In another embodiment, the placenta is transported in a cord blood collection kit substantially as described in pending U.S. Pat. No. 7,147,626, the disclosure of which is incorporated by reference herein. Preferably, the placenta is delivered to the laboratory four to twenty-four hours following delivery. In certain embodiments, the proximal umbilical cord is clamped, preferably within 4-5 cm (centimeter) of the insertion into the placental disc prior to cord blood recovery. In other embodiments, the proximal umbilical cord is clamped after cord blood recovery but prior to further processing of the placenta.

The placenta, prior to cell collection, can be stored under sterile conditions and at either room temperature or at a temperature of 5° C. to 25° C. The placenta may be stored for a period of for a period of four to twenty-four hours, up to forty-eight hours, or longer than forty eight hours, prior to perfusing the placenta to remove any residual cord blood. In one embodiment, the placenta is harvested from between about zero hours to about two hours post-expulsion. The placenta is preferably stored in an anticoagulant solution at a temperature of 5° C. to 25° C. Suitable anticoagulant solutions are well known in the art. For example, a solution of heparin or warfarin sodium can be used. In a preferred embodiment, the anticoagulant solution comprises a solution of heparin (e.g., 1% w/w in 1:1000 solution). The exsanguinated placenta is preferably stored for no more than 36 hours before placental cells are collected.

The mammalian placenta or a part thereof, once collected and prepared generally as above, can be treated in any art-known manner, e.g., can be perfused or disrupted, e.g., digested with one or more tissue-disrupting enzymes, to obtain isolated placental cells.

5.2.3 Physical Disruption and Enzymatic Digestion of Placental Tissue

In one embodiment, stem cells are collected from a mammalian placenta by physical disruption of part of all of the organ. For example, the placenta, or a portion thereof, may be, e.g., crushed, sheared, minced, diced, chopped, macerated or the like. The tissue can then be cultured to obtain a population of isolated placental cells. Typically, the placental tissue is disrupted using, e.g., culture medium, a saline solution, or a stem cell collection.

The placenta can be dissected into components prior to physical disruption and/or enzymatic digestion and stem cell recovery. Isolated placental cells can be obtained from all or a portion of the amniotic membrane, chorion, umbilical cord, placental cotyledons, or any combination thereof, including from a whole placenta. Preferably, isolated placental cells are obtained from placental tissue comprising amnion and chorion. Typically, isolated placental cells can be obtained by disruption of a small block of placental tissue, e.g., a block of placental tissue that is about 1, 2, 3, 4, 5, 6, 7, 8, 9, 10, 20, 30, 40, 50, 60, 70, 80, 90, 100, 200, 300, 400, 500, 600, 700, 800, 900 or about 1000 cubic millimeters in volume. Any method of physical disruption can be used, provided that the method of disruption leaves a plurality, more preferably a majority, and more preferably at least 60%, 70%, 80%, 90%, 95%, 98%, or 99% of the cells in said organ viable, as determined by, e.g., trypan blue exclusion.

The isolated adherent placental cells can generally be collected from a placenta, or portion thereof, at any time within about the first three days post-expulsion, but preferably between about 8 hours and about 18 hours post-expulsion.

In a specific embodiment, the disrupted tissue is cultured in tissue culture medium suitable for the proliferation of isolated placental cells.

In another specific embodiment, isolated placental cells are collected by physical disruption of placental tissue, wherein the physical disruption includes enzymatic digestion, which can be accomplished by use of one or more tissue-digesting enzymes. The placenta, or a portion thereof, may also be physically disrupted and digested with one or more enzymes, and the resulting material then immersed in, or mixed into, a cell collection composition.

A preferred cell collection composition comprises one or more tissue-disruptive enzyme(s). Enzymes that can be used to disrupt placenta tissue include papain, deoxyribonucleases, serine proteases, such as trypsin, chymotrypsin, collagenase, dispase or elastase. Serine proteases may be inhibited by alpha 2 microglobulin in serum and therefore the medium used for digestion is usually serum-free. EDTA and DNase are commonly used in enzyme digestion procedures to increase the efficiency of cell recovery. The digestate is preferably diluted so as to avoid trapping cells within the viscous digest.

Any combination of tissue digestion enzymes can be used. Typical concentrations for digestion using trypsin include, 0.1% to about 2% trypsin, e.g., about 0.25% trypsin. Proteases can be used in combination, that is, two or more proteases in the same digestion reaction, or can be used sequentially in order to liberate placental cells, e.g., placental stem cells and placental multipotent cells. For example, in one embodiment, a placenta, or part thereof, is digested first with an appropriate amount of collagenase I at about 1 to about 2 mg/ml for, e.g., 30 minutes, followed by digestion with trypsin, at a concentration of about 0.25%, for, e.g., 10 minutes, at 37° C. Serine proteases are preferably used consecutively following use of other enzymes.

In another embodiment, the tissue can further be disrupted by the addition of a chelator, e.g., ethylene glycol bis(2-aminoethyl ether)-N,N,N′N′-tetraacetic acid (EGTA) or ethylenediaminetetraacetic acid (EDTA) to the stem cell collection composition comprising the stem cells, or to a solution in which the tissue is disrupted and/or digested prior to isolation of the stem cells with the stem cell collection composition.

Following digestion, the digestate is washed, for example, three times with culture medium, and the washed cells are seeded into culture flasks. The cells are then isolated by differential adherence, and characterized for, e.g., viability, cell surface markers, differentiation, and the like.

It will be appreciated that where an entire placenta, or portion of a placenta comprising both fetal and maternal cells (for example, where the portion of the placenta comprises the chorion or cotyledons), the placental cells isolated can comprise a mix of placental cells derived from both fetal and maternal sources. Where a portion of the placenta that comprises no, or a negligible number of, maternal cells (for example, amnion), the placental cells isolated therefrom will comprise almost exclusively fetal placental cells (that is, placental cells having the genotype of the fetus).

Placental cells, e.g., the placental cells described in Section 5.1, above, can be isolated from disrupted placental tissue by differential trypsinization (see Section 5.2.5, below) followed by culture in one or more new culture containers in fresh proliferation medium, optionally followed by a second differential trypsinization step.

5.2.4 Placental Perfusion

Placental cells, e.g., the placental cells described in Section 5.1, above, can also be obtained by perfusion of the mammalian placenta. Methods of perfusing mammalian placenta to obtain placental cells are disclosed, e.g., in Hariri, U.S. Pat. Nos. 7,045,148 and 7,255,729, in U.S. Patent Application Publication Nos. 2007/0275362 and 2007/0190042, the disclosures of each of which are incorporated herein by reference in their entireties.

Placental cells can be collected by perfusion, e.g., through the placental vasculature, using, e.g., a cell collection composition as a perfusion solution. In one embodiment, a mammalian placenta is perfused by passage of perfusion solution through either or both of the umbilical artery and umbilical vein. The flow of perfusion solution through the placenta may be accomplished using, e.g., gravity flow into the placenta. Preferably, the perfusion solution is forced through the placenta using a pump, e.g., a peristaltic pump. The umbilical vein can be, e.g., cannulated with a cannula, e.g., a TEFLON® or plastic cannula, that is connected to a sterile connection apparatus, such as sterile tubing. The sterile connection apparatus is connected to a perfusion manifold.

In preparation for perfusion, the placenta is preferably oriented (e.g., suspended) in such a manner that the umbilical artery and umbilical vein are located at the highest point of the placenta. The placenta can be perfused by passage of a perfusion fluid through the placental vasculature and surrounding tissue. The placenta can also be perfused by passage of a perfusion fluid into the umbilical vein and collection from the umbilical arteries, or passage of a perfusion fluid into the umbilical arteries and collection from the umbilical vein.

In one embodiment, for example, the umbilical artery and the umbilical vein are connected simultaneously, e.g., to a pipette that is connected via a flexible connector to a reservoir of the perfusion solution. The perfusion solution is passed into the umbilical vein and artery. The perfusion solution exudes from and/or passes through the walls of the blood vessels into the surrounding tissues of the placenta, and is collected in a suitable open vessel from the surface of the placenta that was attached to the uterus of the mother during gestation. The perfusion solution may also be introduced through the umbilical cord opening and allowed to flow or percolate out of openings in the wall of the placenta which interfaced with the maternal uterine wall. Placental cells that are collected by this method, which can be referred to as a “pan” method, are typically a mixture of fetal and maternal cells.

In another embodiment, the perfusion solution is passed through the umbilical veins and collected from the umbilical artery, or is passed through the umbilical artery and collected from the umbilical veins. Placental cells collected by this method, which can be referred to as a “closed circuit” method, are typically almost exclusively fetal.

It will be appreciated that perfusion using the pan method, that is, whereby perfusate is collected after it has exuded from the maternal side of the placenta, results in a mix of fetal and maternal cells. As a result, the cells collected by this method can comprise a mixed population of placental cells, e.g., placental stem cells or placental multipotent cells, of both fetal and maternal origin. In contrast, perfusion solely through the placental vasculature in the closed circuit method, whereby perfusion fluid is passed through one or two placental vessels and is collected solely through the remaining vessel(s), results in the collection of a population of placental cells almost exclusively of fetal origin.

The closed circuit perfusion method can, in one embodiment, be performed as follows. A post-partum placenta is obtained within about 48 hours after birth. The umbilical cord is clamped and cut above the clamp. The umbilical cord can be discarded, or can processed to recover, e.g., umbilical cord stem cells, and/or to process the umbilical cord membrane for the production of a biomaterial. The amniotic membrane can be retained during perfusion, or can be separated from the chorion, e.g., using blunt dissection with the fingers. If the amniotic membrane is separated from the chorion prior to perfusion, it can be, e.g., discarded, or processed, e.g., to obtain stem cells by enzymatic digestion, or to produce, e.g., an amniotic membrane biomaterial, e.g., the biomaterial described in U.S. Application Publication No. 2004/0048796, the disclosure of which is incorporated by reference herein in its entirety. After cleaning the placenta of all visible blood clots and residual blood, e.g., using sterile gauze, the umbilical cord vessels are exposed, e.g., by partially cutting the umbilical cord membrane to expose a cross-section of the cord. The vessels are identified, and opened, e.g., by advancing a closed alligator clamp through the cut end of each vessel. The apparatus, e.g., plastic tubing connected to a perfusion device or peristaltic pump, is then inserted into each of the placental arteries. The pump can be any pump suitable for the purpose, e.g., a peristaltic pump. Plastic tubing, connected to a sterile collection reservoir, e.g., a blood bag such as a 250 mL collection bag, is then inserted into the placental vein. Alternatively, the tubing connected to the pump is inserted into the placental vein, and tubes to a collection reservoir(s) are inserted into one or both of the placental arteries. The placenta is then perfused with a volume of perfusion solution, e.g., about 750 ml of perfusion solution. Cells in the perfusate are then collected, e.g., by centrifugation. In certain embodiments, the placenta is perfused with perfusion solution, e.g., 100-300 mL perfusion solution, to remove residual blood prior to perfusion to collect placental cells, e.g., placental stem cells and/or placental multipotent cells. In another embodiment, the placenta is not perfused with perfusion solution to remove residual blood prior to perfusion to collect placental cells.

In one embodiment, the proximal umbilical cord is clamped during perfusion, and more preferably, is clamped within 4-5 cm (centimeter) of the cord's insertion into the placental disc.

The first collection of perfusion fluid from a mammalian placenta during the exsanguination process is generally colored with residual red blood cells of the cord blood and/or placental blood. The perfusion fluid becomes more colorless as perfusion proceeds and the residual cord blood cells are washed out of the placenta. Generally from 30 to 100 ml (milliliter) of perfusion fluid is adequate to initially exsanguinate the placenta, but more or less perfusion fluid may be used depending on the observed results.

The volume of perfusion liquid used to isolate placental cells may vary depending upon the number of cells to be collected, the size of the placenta, the number of collections to be made from a single placenta, etc. In various embodiments, the volume of perfusion liquid may be from 50 mL to 5000 mL, 50 mL to 4000 mL, 50 mL to 3000 mL, 100 mL to 2000 mL, 250 mL to 2000 mL, 500 mL to 2000 mL, or 750 mL to 2000 mL. Typically, the placenta is perfused with 700-800 mL of perfusion liquid following exsanguination.

The placenta can be perfused a plurality of times over the course of several hours or several days. Where the placenta is to be perfused a plurality of times, it may be maintained or cultured under aseptic conditions in a container or other suitable vessel, and perfused with the cell collection composition, or a standard perfusion solution (e.g., a normal saline solution such as phosphate buffered saline (“PBS”)) with or without an anticoagulant (e.g., heparin, warfarin sodium, coumarin, bishydroxycoumarin), and/or with or without an antimicrobial agent (e.g., β-mercaptoethanol (0.1 mM); antibiotics such as streptomycin (e.g., at 40-100 μg/ml), penicillin (e.g., at 40 U/ml), amphotericin B (e.g., at 0.5 μg/ml). In one embodiment, an isolated placenta is maintained or cultured for a period of time without collecting the perfusate, such that the placenta is maintained or cultured for 1, 2, 3, 4, 5, 6, 7, 8, 9, 10, 11, 12, 13, 14, 15, 16, 17, 18, 19, 20, 21, 22, 23, or 24 hours, or 2 or 3 or more days before perfusion and collection of perfusate. The perfused placenta can be maintained for one or more additional time(s), e.g., 1, 2, 3, 4, 5, 6, 7, 8, 9, 10, 11, 12, 13, 14, 15, 16, 17, 18, 19, 20, 21, 22, 23, 24 or more hours, and perfused a second time with, e.g., 700-800 mL perfusion fluid. The placenta can be perfused 1, 2, 3, 4, 5 or more times, for example, once every 1, 2, 3, 4, 5 or 6 hours. In a preferred embodiment, perfusion of the placenta and collection of perfusion solution, e.g., cell collection composition, is repeated until the number of recovered nucleated cells falls below 100 cells/ml. The perfusates at different time points can be further processed individually to recover time-dependent populations of cells, e.g., stem cells. Perfusates from different time points can also be pooled. In a preferred embodiment, placental cells are collected at a time or times between about 8 hours and about 18 hours post-expulsion.

Perfusion preferably results in the collection of significantly more placental cells than the number obtainable from a mammalian placenta not perfused with said solution, and not otherwise treated to obtain placental cells (e.g., by tissue disruption, e.g., enzymatic digestion). In this context, “significantly more” means at least 10% more. Perfusion yields significantly more placental cells than, e.g., the number of placental cells isolatable from culture medium in which a placenta, or portion thereof, has been cultured.

Placental cells can be isolated from placenta by perfusion with a solution comprising one or more proteases or other tissue-disruptive enzymes. In a specific embodiment, a placenta or portion thereof (e.g., amniotic membrane, amnion and chorion, placental lobule or cotyledon, umbilical cord, or combination of any of the foregoing) is brought to 25-37° C., and is incubated with one or more tissue-disruptive enzymes in 200 mL of a culture medium for 30 minutes. Cells from the perfusate are collected, brought to 4° C., and washed with a cold inhibitor mix comprising 5 mM EDTA, 2 mM dithiothreitol and 2 mM beta-mercaptoethanol. The placental cells are washed after several minutes with a cold (e.g., 4° C.) stem cell collection composition.

5.2.5 Isolation, Sorting, and Characterization of Placental Cells

The isolated placental cells, e.g., the cells described in Section 5.1, above, whether obtained by perfusion or physical disruption, e.g., by enzymatic digestion, can initially be purified from (i.e., be isolated from) other cells by Ficoll gradient centrifugation. Such centrifugation can follow any standard protocol for centrifugation speed, etc. In one embodiment, for example, cells collected from the placenta are recovered from perfusate by centrifugation at 5000×g for 15 minutes at room temperature, which separates cells from, e.g., contaminating debris and platelets. In another embodiment, placental perfusate is concentrated to about 200 ml, gently layered over Ficoll, and centrifuged at about 1100×g for 20 minutes at 22° C., and the low-density interface layer of cells is collected for further processing.

Cell pellets can be resuspended in fresh stem cell collection composition, or a medium suitable for cell maintenance, e.g., stem cell maintenance, for example, IMDM serum-free medium containing 2 U/ml heparin and 2 mM EDTA (GibcoBRL, NY). The total mononuclear cell fraction can be isolated, e.g., using Lymphoprep (Nycomed Pharma, Oslo, Norway) according to the manufacturer's recommended procedure.

Placental cells obtained by perfusion or digestion can, for example, be further, or initially, isolated by differential trypsinization using, e.g., a solution of 0.05% trypsin with 0.2% EDTA (Sigma, St. Louis Mo.). Differential trypsinization is possible because the isolated placental cells, which are tissue culture plastic-adherent, typically detach from the plastic surfaces within about five minutes whereas other adherent populations typically require more than 20-30 minutes incubation. The detached placental cells can be harvested following trypsinization and trypsin neutralization, using, e.g., Trypsin Neutralizing Solution (TNS, Cambrex). In one embodiment of isolation of adherent cells, aliquots of, for example, about 5-10×10⁶ cells are placed in each of several T-75 flasks, preferably fibronectin-coated T75 flasks. In such an embodiment, the cells can be cultured with commercially available Mesenchymal Stem Cell Growth Medium (MSCGM) (Cambrex), and placed in a tissue culture incubator (37° C., 5% CO2). After 10 to 15 days, non-adherent cells are removed from the flasks by washing with PBS. The PBS is then replaced by MSCGM. Flasks are preferably examined daily for the presence of various adherent cell types and in particular, for identification and expansion of clusters of fibroblastoid cells.

The number and type of cells collected from a mammalian placenta can be monitored, for example, by measuring changes in morphology and cell surface markers using standard cell detection techniques such as flow cytometry, cell sorting, immunocytochemistry (e.g., staining with tissue specific or cell-marker specific antibodies) fluorescence activated cell sorting (FACS), magnetic activated cell sorting (MACS), by examination of the morphology of cells using light or confocal microscopy, and/or by measuring changes in gene expression using techniques well known in the art, such as PCR and gene expression profiling. These techniques can be used, too, to identify cells that are positive for one or more particular markers. For example, using antibodies to CD34−, one can determine, using the techniques above, whether a cell comprises a detectable amount of CD34; if so, the cell is CD34+. Likewise, if a cell produces enough OCT-4 RNA to be detectable by RT-PCR, or significantly more OCT-4 RNA than an adult cell, the cell is OCT-4+. Antibodies to cell surface markers (e.g., CD markers such as CD34) and the sequence of stem cell-specific genes, such as OCT-4, are well-known in the art.

Placental cells, particularly cells that have been isolated by Ficoll separation, differential adherence, or a combination of both, may be sorted using a fluorescence activated cell sorter (FACS). Fluorescence activated cell sorting (FACS) is a well-known method for separating particles, including cells, based on the fluorescent properties of the particles (Kamarch, 1987, Methods Enzymol, 151:150-165). Laser excitation of fluorescent moieties in the individual particles results in a small electrical charge allowing electromagnetic separation of positive and negative particles from a mixture. In one embodiment, cell surface marker-specific antibodies or ligands are labeled with distinct fluorescent labels. Cells are processed through the cell sorter, allowing separation of cells based on their ability to bind to the antibodies used. FACS sorted particles may be directly deposited into individual wells of 96-well or 384-well plates to facilitate separation and cloning.

In one sorting scheme, cells from placenta, e.g., PDACs are sorted on the basis of expression of one or more of the markers CD34, CD38, CD44, CD45, CD73, CD105, OCT-4 and/or HLA-G. This can be accomplished in connection with procedures to select such cells on the basis of their adherence properties in culture. For example, tissue culture plastic adherence selection can be accomplished before or after sorting on the basis of marker expression. In one embodiment, for example, cells are sorted first on the basis of their expression of CD34; CD34− cells are retained, and CD34− cells that are additionally CD200+ and HLA-G− are separated from all other CD34− cells. In another embodiment, cells from placenta are sorted based on their expression of markers CD200 and/or HLA-G; for example, cells displaying CD200 and lacking HLA-G are isolated for further use. Cells that express, e.g., CD200 and/or lack, e.g., HLA-G can, in a specific embodiment, be further sorted based on their expression of CD73 and/or CD105, or epitopes recognized by antibodies SH2, SH3 or SH4, or lack of expression of CD34, CD38 or CD45. For example, in another embodiment, placental cells are sorted by expression, or lack thereof, of CD200, HLA-G, CD73, CD105, CD34, CD38 and CD45, and placental cells that are CD200+, HLA-G−, CD73+, CD105+, CD34−, CD38− and CD45− are isolated from other placental cells for further use.

In specific embodiments of any of the above embodiments of sorted placental cells, at least 50%, 60%, 70%, 80%, 90% or 95% of the cells in a cell population remaining after sorting are said isolated placental cells. Placental cells can be sorted by one or more of any of the markers described in Section 5.1, above.

In a specific embodiment, for example, placental cells that are (1) adherent to tissue culture plastic, and (2) CD10+, CD34− and CD105+ are sorted from (i.e., isolated from) other placental cells. In another specific embodiment, placental cells that are (1) adherent to tissue culture plastic, and (2) CD10+, CD34−, CD105+ and CD200+ are sorted from (i.e., isolated from) other placental cells. In another specific embodiment, placental cells that are (1) adherent to tissue culture plastic, and (2) CD10+, CD34−, CD45−, CD90+, CD105+ and CD200+ are sorted from (i.e., isolated from) other placental cells.

With respect to nucleotide sequence-based detection of placental cells, sequences for the markers listed herein are readily available in publicly-available databases such as GenBank or EMBL.

With respect to antibody-mediated detection and sorting of placental cells, e.g., placental stem cells or placental multipotent cells, any antibody, specific for a particular marker, can be used, in combination with any fluorophore or other label suitable for the detection and sorting of cells (e.g., fluorescence-activated cell sorting). Antibody/fluorophore combinations to specific markers include, but are not limited to, fluorescein isothiocyanate (FITC) conjugated monoclonal antibodies against HLA-G− (available from Serotec, Raleigh, N.C.), CD10 (available from BD Immunocytometry Systems, San Jose, Calif.), CD44 (available from BD Biosciences Pharmingen, San Jose, Calif.), and CD105 (available from R&D Systems Inc., Minneapolis, Minn.); phycoerythrin (PE) conjugated monoclonal antibodies against CD44, CD200, CD117, and CD13 (BD Biosciences Pharmingen); phycoerythrin-Cy7 (PE Cy7) conjugated monoclonal antibodies against CD33 and CD10 (BD Biosciences Pharmingen); allophycocyanin (APC) conjugated streptavidin and monoclonal antibodies against CD38 (BD Biosciences Pharmingen); and Biotinylated CD90 (BD Biosciences Pharmingen). Other antibodies that can be used include, but are not limited to, CD133-APC (Miltenyi), KDR-Biotin (CD309, Abcam), CytokeratinK-Fitc (Sigma or Dako), HLA ABC-Fitc (BD), HLA DR,DQ,DP-PE (BD), β-2-microglobulin-PE (BD), CD80-PE (BD) and CD86-APC (BD). Other antibody/label combinations that can be used include, but are not limited to, CD45-PerCP (peridin chlorophyll protein); CD44-PE; CD19-PE; CD10-F (fluorescein); HLA-G-F and 7-amino-actinomycin-D (7-AAD); HLA-ABC-F; and the like. This list is not exhaustive, and other antibodies from other suppliers are also commercially available.

The isolated placental cells provided herein can be assayed for CD117 or CD133 using, for example, phycoerythrin-Cy5 (PE Cy5) conjugated streptavidin and biotin conjugated monoclonal antibodies against CD117 or CD133; however, using this system, the cells can appear to be positive for CD117 or CD133, respectively, because of a relatively high background.

The isolated placental cells can be labeled with an antibody to a single marker and detected and/sorted. Placental cells can also be simultaneously labeled with multiple antibodies to different markers.

In another embodiment, magnetic beads can be used to separate cells. The cells may be sorted using a magnetic activated cell sorting (MACS) technique, a method for separating particles based on their ability to bind magnetic beads (0.5-100 μm diameter). A variety of useful modifications can be performed on the magnetic microspheres, including covalent addition of antibody that specifically recognizes a particular cell surface molecule or hapten. The beads are then mixed with the cells to allow binding. Cells are then passed through a magnetic field to separate out cells having the specific cell surface marker. In one embodiment, these cells can then isolated and re-mixed with magnetic beads coupled to an antibody against additional cell surface markers. The cells are again passed through a magnetic field, isolating cells that bound both the antibodies. Such cells can then be diluted into separate dishes, such as microtiter dishes for clonal isolation.

Isolated placental cells can also be characterized and/or sorted based on cell morphology and growth characteristics. For example, isolated placental cells can be characterized as having, and/or selected on the basis of, e.g., a fibroblastoid appearance in culture. The isolated placental cells can also be characterized as having, and/or be selected, on the basis of their ability to form embryoid-like bodies. In one embodiment, for example, placental cells that are fibroblastoid in shape, express CD73 and CD105, and produce one or more embryoid-like bodies in culture are isolated from other placental cells. In another embodiment, OCT-4+ placental cells that produce one or more embryoid-like bodies in culture are isolated from other placental cells.

In another embodiment, isolated placental cells can be identified and characterized by a colony forming unit assay. Colony forming unit assays are commonly known in the art, such as MesenCult™ medium (Stem Cell Technologies, Inc., Vancouver British Columbia).

The isolated placental cells can be assessed for viability, proliferation potential, and longevity using standard techniques known in the art, such as trypan blue exclusion assay, fluorescein diacetate uptake assay, propidium iodide uptake assay (to assess viability); and thymidine uptake assay, MTT (3-(4,5-Dimethylthiazol-2-yl)-2,5-diphenyltetrazolium bromide) cell proliferation assay (to assess proliferation). Longevity may be determined by methods well known in the art, such as by determining the maximum number of population doubling in an extended culture.

Isolated placental cells, e.g., the isolated placental cells described in Section 5.1, above, can also be separated from other placental cells using other techniques known in the art, e.g., selective growth of desired cells (positive selection), selective destruction of unwanted cells (negative selection); separation based upon differential cell agglutinability in the mixed population as, for example, with soybean agglutinin; freeze-thaw procedures; filtration; conventional and zonal centrifugation; centrifugal elutriation (counter-streaming centrifugation); unit gravity separation; countercurrent distribution; electrophoresis; and the like.

5.2.6 Populations of Isolated Placental Cells

Also provided herein are populations of isolated placental cells, e.g., the isolated placental cells described in Section 5.1, above, useful in the methods and compositions described herein. Populations of isolated placental cells can be isolated directly from one or more placentas; that is, the cell population can be a population of placental cells comprising the isolated placental cells, wherein the isolated placental cells are obtained from, or contained within, perfusate, or obtained from, or contained within, disrupted placental tissue, e.g., placental tissue digestate (that is, the collection of cells obtained by enzymatic digestion of a placenta or part thereof). The isolated placental cells described herein can also be cultured and expanded to produce populations of the isolated placental cells. Populations of placental cells comprising the isolated placental cells can also be cultured and expanded to produce placental cell populations.

Placental cell populations useful in the methods of treatment provided herein comprise the isolated placental cells, for example, the isolated placental cells as described in Section 5.1 herein. In various embodiments, at least 10%, 20%, 30%, 40%, 50%, 60%, 70%, 80%, 90%, 95%, or 99% of the cells in a placental cell population are the isolated placental cells. That is, a population of the isolated placental cells can comprise, e.g., as much as 1%, 5%, 10%, 20%, 30%, 40%, 50%, 60%, 70%, 80%, 90% cells that are not the isolated placental cells.

Isolated placental cell populations useful in the methods and compositions described herein can be produced by, e.g., selecting isolated placental cells, whether derived from enzymatic digestion or perfusion, that express particular markers and/or particular culture or morphological characteristics. In one embodiment, for example, provided herein is a method of producing a cell population by selecting placental cells that (a) adhere to a substrate, and (b) express CD200 and lack expression of HLA-G; and isolating said cells from other cells to form a cell population. In another embodiment, a cell population is produced by selecting placental cells that express CD200 and lack expression of HLA-G, and isolating said cells from other cells to form a cell population. In another embodiment, a cell population is produced by selecting placental cells that (a) adhere to a substrate, and (b) express CD73, CD105, and CD200; and isolating said cells from other cells to form a cell population. In another embodiment, a cell population is produced by identifying placental cells that express CD73, CD105, and CD200, and isolating said cells from other cells to form a cell population. In another embodiment, a cell population is produced by selecting placental cells that (a) adhere to a substrate and (b) express CD200 and OCT-4; and isolating said cells from other cells to form a cell population. In another embodiment, a cell population is produced by selecting placental cells that express CD200 and OCT-4, and isolating said cells from other cells to form a cell population. In another embodiment, a cell population is produced by selecting placental cells that (a) adhere to a substrate, (b) express CD73 and CD105, and (c) facilitate the formation of one or more embryoid-like bodies in a population of placental cells comprising said stem cell when said population is cultured under conditions that allow for the formation of an embryoid-like body; and isolating said cells from other cells to form a cell population. In another embodiment, a cell population is produced by selecting placental cells that express CD73 and CD105, and facilitate the formation of one or more embryoid-like bodies in a population of placental cells comprising said stem cell when said population is cultured under conditions that allow for the formation of an embryoid-like body, and isolating said cells from other cells to form a cell population. In another embodiment, a cell population is produced by selecting placental cells that (a) adhere to a substrate, and (b) express CD73 and CD105, and lack expression of HLA-G; and isolating said cells from other cells to form a cell population. In another embodiment, a cell population is produced by selecting placental cells that express CD73 and CD105 and lack expression of HLA-G, and isolating said cells from other cells to form a cell population. In another embodiment, the method of producing a cell population comprises selecting placental cells that (a) adhere to a substrate, (b) express OCT-4, and (c) facilitate the formation of one or more embryoid-like bodies in a population of placental cells comprising said stem cell when said population is cultured under conditions that allow for the formation of an embryoid-like body; and isolating said cells from other cells to form a cell population. In another embodiment, a cell population is produced by selecting placental cells that express OCT-4, and facilitate the formation of one or more embryoid-like bodies in a population of placental cells comprising said stem cell when said population is cultured under conditions that allow for the formation of an embryoid-like body, and isolating said cells from other cells to form a cell population.

In another embodiment, a cell population is produced by selecting placental cells that (a) adhere to a substrate, and (b) express CD10 and CD105, and do not express CD34; and isolating said cells from other cells to form a cell population. In another embodiment, a cell population is produced by selecting placental cells that express CD10 and CD105, and do not express CD34, and isolating said cells from other cells to form a cell population. In another embodiment, a cell population is produced by selecting placental cells that (a) adhere to a substrate, and (b) express CD10, CD105, and CD200, and do not express CD34; and isolating said cells from other cells to form a cell population. In another embodiment, a cell population is produced by selecting placental cells that express CD10, CD105, and CD200, and do not express CD34, and isolating said cells from other cells to form a cell population. In another specific embodiment, a cell population is produced by selecting placental cells that (a) adhere to a substrate, and (b) express CD10, CD90, CD105 and CD200, and do not express CD34 and CD45; and isolating said cells from other cells to form a cell population. In another specific embodiment, a cell population is produced by selecting placental cells that express CD10, CD90, CD105 and CD200, and do not express CD34 and CD45, and isolating said cells from other cells to form a cell population.

Selection of cell populations comprising placental cells having any of the marker combinations described in Section 5.1, above, can be isolated or obtained in similar fashion.

In any of the above embodiments, selection of the isolated cell populations can additionally comprise selecting placental cells that express ABC-p (a placenta-specific ABC transporter protein; see, e.g., Allikmets et al., Cancer Res. 58(23):5337-9 (1998)). The method can also comprise selecting cells exhibiting at least one characteristic specific to, e.g., a mesenchymal stem cell, for example, expression of CD44, expression of CD90, or expression of a combination of the foregoing.

In the above embodiments, the substrate can be any surface on which culture and/or selection of cells, e.g., isolated placental cells, can be accomplished. Typically, the substrate is plastic, e.g., tissue culture dish or multiwell plate plastic. Tissue culture plastic can be coated with a biomolecule, e.g., laminin or fibronectin.

Cells, e.g., isolated placental cells, can be selected for a placental cell population by any means known in the art of cell selection. For example, cells can be selected using an antibody or antibodies to one or more cell surface markers, for example, in flow cytometry or FACS. Selection can be accomplished using antibodies in conjunction with magnetic beads. Antibodies that are specific for certain stem cell-related markers are known in the art. For example, antibodies to OCT-4 (Abcam, Cambridge, Mass.), CD200 (Abcam), HLA-G− (Abcam), CD73 (BD Biosciences Pharmingen, San Diego, Calif.), CD105 (Abcam; BioDesign International, Saco, Me.), etc. Antibodies to other markers are also available commercially, e.g., CD34, CD38 and CD45 are available from, e.g., StemCell Technologies or BioDesign International.

The isolated placental cell populations can comprise placental cells that are not stem cells, or cells that are not placental cells.

The isolated cell populations comprising placental derived adherent cells described herein can comprise a second cell type, e.g., placental cells that are not placental derived adherent cells, or, e.g., cells that are not placental cells. For example, an isolated population of placental derived adherent cells can comprise, e.g., can be combined with, a population of a second type of cells, wherein said second type of cell are, e.g., embryonic stem cells, blood cells (e.g., placental blood, placental blood cells, umbilical cord blood, umbilical cord blood cells, peripheral blood, peripheral blood cells, nucleated cells from placental blood, umbilical cord blood, or peripheral blood, and the like), stem cells isolated from blood (e.g., stem cells isolated from placental blood, umbilical cord blood or peripheral blood), nucleated cells from placental perfusate, e.g., total nucleated cells from placental perfusate; umbilical cord stem cells, populations of blood-derived nucleated cells, bone marrow-derived mesenchymal stromal cells, bone marrow-derived mesenchymal stem cells, bone marrow-derived hematopoietic stem cells, crude bone marrow, adult (somatic) stem cells, populations of stem cells contained within tissue, cultured cells, e.g., cultured stem cells, populations of fully-differentiated cells (e.g., chondrocytes, fibroblasts, amniotic cells, osteoblasts, muscle cells, cardiac cells, etc.), pericytes, and the like. In a specific embodiment, a population of cells comprising placental derived adherent cells comprises placental stem cells or stem cells from umbilical cord. In certain embodiments in which the second type of cell is blood or blood cells, erythrocytes have been removed from the population of cells.

In a specific embodiment, the second type of cell is a hematopoietic stem cell. Such hematopoietic stem cells can be, for example, contained within unprocessed placental, umbilical cord blood or peripheral blood; in total nucleated cells from placental blood, umbilical cord blood or peripheral blood; in an isolated population of CD34+ cells from placental blood, umbilical cord blood or peripheral blood; in unprocessed bone marrow; in total nucleated cells from bone marrow; in an isolated population of CD34+ cells from bone marrow, or the like.

In another embodiment, an isolated population of placental derived adherent cells is combined with a plurality of adult or progenitor cells from the vascular system. In various embodiments, the cells are endothelial cells, endothelial progenitor cells, myocytes, cardiomyocytes, pericytes, angioblasts, myoblasts or cardiomyoblasts.

In a another embodiment, the second cell type is a non-embryonic cell type manipulated in culture in order to express markers of pluripotency and functions associated with embryonic stem cells

In specific embodiments of the above isolated populations of placental derived adherent cells, either or both of the placental derived adherent cells and cells of a second type are autologous, or are allogeneic, to an intended recipient of the cells.

In another specific embodiment, the composition comprises placental derived adherent cells, and embryonic stem cells. In another specific embodiment, the composition comprises placental derived adherent cells and mesenchymal stromal or stem cells, e.g., bone marrow-derived mesenchymal stromal or stem cells. In another specific embodiment, the composition comprises bone marrow-derived hematopoietic stem cells. In another specific embodiment, the composition comprises placental derived adherent cells and hematopoietic progenitor cells, e.g., hematopoietic progenitor cells from bone marrow, fetal blood, umbilical cord blood, placental blood, and/or peripheral blood. In another specific embodiment, the composition comprises placental derived adherent cells and somatic stem cells. In a more specific embodiment, said somatic stem cell is a neural stem cell, a hepatic stem cell, a pancreatic stem cell, an endothelial stem cell, a cardiac stem cell, or a muscle stem cell.

In other specific embodiments, the second type of cells comprise about, at least, or no more than, 10%, 15%, 20%, 25%, 30%, 35%, 40%, 45%, or 50% of cells in said population. In other specific embodiments, the PDAC in said composition comprise at least 50%, 55%, 60%, 65%, 70%, 75%, 80%, 85% or 90% of cells in said composition. In other specific embodiments, the placental derived adherent cells comprise about, at least, or no more than, 10%, 15%, 20%, 25%, 30%, 35%, 40%, or 45% of cells in said population.

Cells in an isolated population of placental derived adherent cells can be combined with a plurality of cells of another type, e.g., with a population of stem cells, in a ratio of about 100,000,000:1, 50,000,000:1, 20,000,000:1, 10,000,000:1, 5,000,000:1, 2,000,000:1, 1,000,000:1, 500,000:1, 200,000:1, 100,000:1, 50,000:1, 20,000:1, 10,000:1, 5,000:1, 2,000:1, 1,000:1, 500:1, 200:1, 100:1, 50:1, 20:1, 10:1, 5:1, 2:1, 1:1; 1:2; 1:5; 1:10; 1:100; 1:200; 1:500; 1:1,000; 1:2,000; 1:5,000; 1:10,000; 1:20,000; 1:50,000; 1:100,000; 1:500,000; 1:1,000,000; 1:2,000,000; 1:5,000,000; 1:10,000,000; 1:20,000,000; 1:50,000,000; or about 1:100,000,000, comparing numbers of total nucleated cells in each population. Cells in an isolated population of placental derived adherent cells can be combined with a plurality of cells of a plurality of cell types, as well.

In other embodiments, a population of the placental cells described herein, e.g., the PDACs described above, are combined with osteogenic placental adherent cells (OPACs), e.g., the OPACs described in patent application Ser. No. 12/546,556, filed Aug. 24, 2009, entitled “Methods and Compositions for Treatment of Bone Defects With Placental Stem Cells,” or combined with amnion-derived angiogenic cells (AMDACs), e.g., the AMDACs described in U.S. patent application Ser. No. 12/622,352, entitled “Amnion Derived Angiogenic Cells”, the disclosure of which is hereby incorporated by reference in its entirety.

### 5.3 Compositions Comprising Isolated Placental Cells

The placental cells described herein, e.g., in Section 5.1, can be combined with any physiologically-acceptable or medically-acceptable compound, composition or device for use in the methods and compositions described herein. Compositions useful in the methods of treatment provided herein can comprise any one or more of the placental cells described herein. In certain embodiments, the composition is a pharmaceutically-acceptable composition, e.g., a composition comprising placental cells in a pharmaceutically-acceptable carrier.

In certain embodiments, a composition comprising the isolated placental cells additionally comprises a matrix, e.g., a decellularized matrix or a synthetic matrix. In another specific embodiment, said matrix is a three-dimensional scaffold. In another specific embodiment, said matrix comprises collagen, gelatin, laminin, fibronectin, pectin, ornithine, or vitronectin. In another ore specific embodiment, the matrix is an amniotic membrane or an amniotic membrane-derived biomaterial. In another specific embodiment, said matrix comprises an extracellular membrane protein. In another specific embodiment, said matrix comprises a synthetic compound. In another specific embodiment, said matrix comprises a bioactive compound. In another specific embodiment, said bioactive compound is a growth factor, cytokine, antibody, or organic molecule of less than 5,000 daltons.

In another embodiment, a composition useful in the methods of treatment provided herein comprises medium conditioned by any of the foregoing placental cells, or any of the foregoing placental cell populations.

5.3.1 Cryopreserved Isolated Placental Cells

The isolated placental cell populations useful in the methods and compositions described herein can be preserved, for example, cryopreserved for later use. Methods for cryopreservation of cells, such as stem cells, are well known in the art. Isolated placental cell populations can be prepared in a form that is easily administrable to an individual, e.g., an isolated placental cell population that is contained within a container that is suitable for medical use. Such a container can be, for example, a syringe, sterile plastic bag, flask, jar, or other container from which the isolated placental cell population can be easily dispensed. For example, the container can be a blood bag or other plastic, medically-acceptable bag suitable for the intravenous administration of a liquid to a recipient. The container, in certain embodiments, is one that allows for cryopreservation of the combined cell population.

The cryopreserved isolated placental cell population can comprise isolated placental cell derived from a single donor, or from multiple donors. The isolated placental cell population can be completely HLA-matched to an intended recipient, or partially or completely HLA-mismatched.

Thus, in one embodiment, isolated placental cells can be used in the methods and described herein in the form of a composition comprising a tissue culture plastic-adherent placental cell population in a container. In a specific embodiment, the isolated placental cells are cryopreserved. In another specific embodiment, the container is a bag, flask, or jar. In another specific embodiment, said bag is a sterile plastic bag. In another specific embodiment, said bag is suitable for, allows or facilitates intravenous administration of said isolated placental cell population, e.g., by intravenous infusion. The bag can comprise multiple lumens or compartments that are interconnected to allow mixing of the isolated placental cells and one or more other solutions, e.g., a drug, prior to, or during, administration. In another specific embodiment, the composition comprises one or more compounds that facilitate cryopreservation of the combined cell population. In another specific embodiment, said isolated placental cell population is contained within a physiologically-acceptable aqueous solution. In another specific embodiment, said physiologically-acceptable aqueous solution is a 0.9% NaCl solution. In another specific embodiment, said isolated placental cell population comprises placental cells that are HLA-matched to a recipient of said cell population. In another specific embodiment, said combined cell population comprises placental cells that are at least partially HLA-mismatched to a recipient of said cell population. In another specific embodiment, said isolated placental cells are derived from a plurality of donors.

In certain embodiments, the isolated placental cells in the container are isolated CD10+, CD34−, CD105+ placental cells, wherein said cells have been cryopreserved, and are contained within a container. In a specific embodiment, said CD10+, CD34−, CD105+ placental cells are also CD200+. In another specific embodiment, said CD10+, CD34−, CD105+, CD200+ placental cells are also CD45− or CD90+. In another specific embodiment, said CD10+, CD34−, CD105+, CD200+ placental cells are also CD45− and CD90+. In another specific embodiment, the CD34−, CD10+, CD105+ placental cells are additionally one or more of CD13+, CD29+, CD33+, CD38−, CD44+, CD45−, CD54+, CD62E−, CD62L−, CD62P−, SH3+ (CD73+), SH4+ (CD73+), CD80−, CD86−, CD90+, SH2+ (CD105+), CD106NCAM+, CD117−, CD144/VE-cadherindim, CD184/CXCR4−, CD200+, CD133−, OCT-4+, SSEA3−, SSEA4−, ABC-p+, KDR− (VEGFR2−), HLA-A,B,C+, HLA-DP,DQ,DR−, HLA-G−, or Programmed Death-1 Ligand (PDL1)+, or any combination thereof. In another specific embodiment, the CD34−, CD10+, CD105+ placental cells are additionally CD13+, CD29+, CD33+, CD38−, CD44+, CD45−, CD54/ICAM+, CD62E−, CD62L−, CD62P−, SH3+ (CD73+), SH4+ (CD73+), CD80−, CD86−, CD90+, SH2+ (CD105+), CD106/VCAM+, CD117−, CD144/VE-cadherindim, CD184/CXCR4−, CD200+, CD133−, OCT-4+, SSEA3−, SSEA4−, ABC-p+, KDR− (VEGFR2−), HLA-A,B,C+, HLA-DP,DQ,DR−, HLA-G−, and Programmed Death-1 Ligand (PDL1)+.

In certain other embodiments, the above-referenced isolated placental cells are isolated CD200+, HLA-G− placental cells, wherein said cells have been cryopreserved, and are contained within a container. In another embodiment, the isolated placental cells are CD73+, CD105+, CD200+ cells that have been cryopreserved, and are contained within a container. In another embodiment, the isolated placental cells are CD200+, OCT-4+ stem cells that have been cryopreserved, and are contained within a container. In another embodiment, the isolated placental cells are CD73+, CD105+ cells that have been cryopreserved, and are contained within a container, and wherein said isolated placental cells facilitate the formation of one or more embryoid-like bodies when cultured with a population of placental cells under conditions that allow for the formation of embryoid-like bodies. In another embodiment, the isolated placental cells are CD73+, CD105+, HLA-G− cells that have been cryopreserved, and are contained within a container. In another embodiment, the isolated placental cells are OCT-4+ placental cells that have been cryopreserved, and are contained within a container, and wherein said cells facilitate the formation of one or more embryoid-like bodies when cultured with a population of placental cells under conditions that allow for the formation of embryoid-like bodies.

In another specific embodiment, the above-referenced isolated placental cells are placental stem cells or placental multipotent cells that are CD34−, CD10+ and CD105+ as detected by flow cytometry (e.g., PDACs). In another specific embodiment, the isolated CD34−, CD10+, CD105+ placental cells have the potential to differentiate into cells of a neural phenotype, cells of an osteogenic phenotype, or cells of a chondrogenic phenotype. In another specific embodiment, the isolated CD34−, CD10+, CD105+ placental cells are additionally CD200+. In another specific embodiment, the isolated CD34−, CD10+, CD105+ placental cells are additionally CD90+ or CD45−, as detected by flow cytometry. In another specific embodiment, the isolated CD34−, CD10+, CD105+ placental cells are additionally CD90+ or CD45−, as detected by flow cytometry. In another specific embodiment, the CD34−, CD10+, CD105+, CD200+ placental cells are additionally CD90+ or CD45−, as detected by flow cytometry. In another specific embodiment, the CD34−, CD10+, CD105+, CD200+ cells are additionally CD90+ and CD45−, as detected by flow cytometry. In another specific embodiment, the CD34−, CD10+, CD105+, CD200+, CD90+, CD45− cells are additionally CD80− and CD86−, as detected by flow cytometry. In another specific embodiment, the CD34−, CD10+, CD105+ cells are additionally one or more of CD29+, CD38−, CD44+, CD54+, CD80−, CD86−, SH3+ or SH4+. In another specific embodiment, the cells are additionally CD44+. In a specific embodiment of any of the isolated CD34−, CD10+, CD105+ placental cells above, the cells are additionally one or more of CD117−, CD133−, KDR− (VEGFR2−), HLA-A,B,C+, HLA-DP,DQ,DR−, and/or PDL1+.

In a specific embodiment of any of the foregoing cryopreserved isolated placental cells, said container is a bag. In various specific embodiments, said container comprises about, at least, or at most 1×10⁶ said isolated placental cells, 5×10⁶ said isolated placental cells, 1×10⁷ said isolated placental cells, 5×10⁷ said isolated placental cells, 1×10⁸ said isolated placental cells, 5×10⁸ said isolated placental cells, 1×10⁹ said isolated placental cells, 5×10⁹ said isolated placental cells, 1×10¹⁰ said isolated placental cells, or 1×10¹⁰ said isolated placental cells. In other specific embodiments of any of the foregoing cryopreserved populations, said isolated placental cells have been passaged about, at least, or no more than 5 times, no more than 10 times, no more than 15 times, or no more than 20 times. In another specific embodiment of any of the foregoing cryopreserved isolated placental cells, said isolated placental cells have been expanded within said container.

In certain embodiments, a single unit dose of placental derived adherent cells can comprise, in various embodiments, about, at least, or no more than 1×10³, 3×10³, 5×10³, 1×10⁴, 3×10⁴, 5×10⁴, 1×10⁵, 3×10⁵, 5×10⁵, 1×10⁶, 3×10⁶, 5×10⁶, 1×10⁷, 3×10⁷, 5×10⁷, 1×10⁸, 3×10⁸, 5×10⁸, 1×10⁹, 5×10⁹, or 1×10¹⁰ placental cells. In certain embodiments, a single unit dose of placental derived adherent cells can comprise between 1×10³ to 3×10³, 3×10³ to 5×10³, 5×10³ to 1×10⁴, 1×10⁴ to 3×10⁴, 3×10⁴ to 5×10⁴, 5×10⁴ to 1×10⁵, 1×10⁵ to 3×10⁵, 3×10⁵ to 5×10⁵, 5×10⁵ to 1×10⁶, 1×10⁶ to 3×10⁶, 3×10⁶ to 5×10⁶, 5×10⁶ to 1×10⁷, 1×10⁷ to 3×10⁷, 3×10⁷ to 5×10⁷, 5×10⁷ to 1×10⁸, 1×10⁸ to 3×10⁸, 3×10⁸ to 5×10⁸, 5×10⁸ to 1×10⁹, 1×10⁹ to 5×10⁹, or 5×10⁹ to 1×10¹⁰ placental cells. In certain embodiments, the pharmaceutical compositions provided herein comprises populations of placental derived adherent cells, that comprise 50% viable cells or more (that is, at least 50% of the cells in the population are functional or living). Preferably, at least 60% of the cells in the population are viable. More preferably, at least 70%, 80%, 90%, 95%, or 99% of the cells in the population in the pharmaceutical composition are viable.

5.3.2 Pharmaceutical Compositions

Populations of isolated placental cells, e.g., PDACs, or populations of cells comprising the isolated placental cells, can be formulated into pharmaceutical compositions for use in vivo, e.g., in the methods of treatment provided herein. Such pharmaceutical compositions comprise a population of isolated placental cells, or a population of cells comprising isolated placental cells, in a pharmaceutically-acceptable carrier, e.g., a saline solution or other accepted physiologically-acceptable solution for in vivo administration. Pharmaceutical compositions comprising the isolated placental cells described herein can comprise any, or any combination, of the isolated placental cell populations, or isolated placental cells, described elsewhere herein. The pharmaceutical compositions can comprise fetal, maternal, or both fetal and maternal isolated placental cells. The pharmaceutical compositions provided herein can further comprise isolated placental cells obtained from a single individual or placenta, or from a plurality of individuals or placentae.

The pharmaceutical compositions provided herein can comprise any number of isolated placental cells. For example, a single unit dose of placental derived adherent cells can comprise about, at least, or no more than 1×10³, 3×10³, 5×10³, 1×10⁴, 3×10⁴, 5×10⁴, 1×10⁵, 3×10⁵, 5×10⁵, 1×10⁶, 3×10⁶, 5×10⁶, 1×10⁷, 3×10⁷, 5×10⁷, 1×10⁸, 3×10⁸, 5×10⁸, 1×10⁹, 5×10⁹, or 1×10¹⁰ placental cells or between 1×10³ to 3×10³, 3×10³ to 5×10³, 5×10³ to 1×10⁴, 1×10⁴ to 3×10⁴, 3×10⁴ to 5×10⁴, 5×10⁴ to 1×10⁵, 1×10⁵ to 3×10⁵, 3×10⁵ to 5×10⁵, 5×10⁵ to 1×10⁶, 1×10⁶ to 3×10⁶, 3×10⁶ to 5×10⁶, 5×10⁶ to 1×10⁷, 1×10⁷ to 3×10⁷, 3×10⁷ to 5×10⁷, 5×10⁷ to 1×10⁸, 1×10⁸ to 3×10⁸, 3×10⁸ to 5×10⁸, 5×10⁸ to 1×10⁹, 1×10⁹ to 5×10⁹, or 5×10⁹ to 1×10¹⁰ placental cells.

In certain embodiments, the pharmaceutical compositions provided herein are administered to a subject having diabetic foot ulcer once. In certain embodiments, the pharmaceutical compositions provided herein are administered to a subject having diabetic foot ulcer on multiple occasions, e.g., twice, three times, four times, five times, six times, seven times, eight times, nine times, ten times, or more than ten times. Intervals between dosages can be weekly, bi-weekly, monthly, bi-monthly or yearly. Intervals can also be irregular. Doses of placental stem cells administered according to such regimens include, but are not limited to, 1×10³, 3×10³, 5×10³, 1×10⁴, 3×10⁴, 5×10⁴, 1×10⁵, 3×10⁵, 5×10⁵, 1×10⁶, 3×10⁶, 5×10⁶, 1×10⁷, 3×10⁷, 5×10⁷, 1×10⁸, 3×10⁸, 5×10⁸, 1×10⁹, 5×10⁹, or 1×10¹⁰ placental cells or between 1×10³ to 3×10³, 3×10³ to 5×10³, 5×10³ to 1×10⁴, 1×10⁴ to 3×10⁴, 3×10⁴ to 5×10⁴, 5×10⁴ to 1×10⁵, 1×10⁵ to 3×10⁵, 3×10⁵ to 5×10⁵, 5×10⁵ to 1×10⁶, 1×10⁶ to 3×10⁶, 3×10⁶ to 5×10⁶, 5×10⁶ to 1×10⁷, 1×10⁷ to 3×10⁷, 3×10⁷ to 5×10⁷, 5×10⁷ to 1×10⁸, 1×10⁸ to 3×10⁸, 3×10⁸ to 5×10⁸, 5×10⁸ to 1×10⁹, 1×10⁹ to 5×10⁹, or 5×10⁹ to 1×10¹⁰ placental stem cells. In a specific embodiment, the dose of placental stem cells in a pharmaceutical composition is 1×10³ placental stem cells. In another specific embodiment, the dose of placental stem cells in a pharmaceutical composition is 3×10³ placental stem cells. In another specific embodiment, the dose of placental stem cells in a pharmaceutical composition is 3×10⁴ placental stem cells. In another specific embodiment, the dose of placental stem cells in a pharmaceutical composition is 3×10⁵ placental stem cells. In another specific embodiment, the dose of placental stem cells in a pharmaceutical composition is 1×10⁶ placental stem cells. In another specific embodiment, the dose of placental stem cells in a pharmaceutical composition is 3×10⁶ placental stem cells. In another specific embodiment, the dose of placental stem cells in a pharmaceutical composition is 3×10⁷ placental stem cells.

In certain embodiments, a pharmaceutical composition comprising placental stem cells (e.g., CD10+, CD105+, CD200+, CD34− placental stem cells) is administered to a subject having diabetic foot ulcer once as a single dose. In certain embodiments, a pharmaceutical composition comprising placental stem cells (e.g., CD10+, CD105+, CD200+, CD34− placental stem cells) is administered to a subject having diabetic foot ulcer as a single dose followed by a second dose about 1 week later. In certain embodiments, a pharmaceutical composition comprising placental stem cells (e.g., CD10+, CD105+, CD200+, CD34− placental stem cells) is administered to a subject having diabetic foot ulcer as a single dose followed by a second dose about 1 week later and a third dose about one week after that (i.e., about two weeks after the initial administration). Doses of placental stem cells administered according to such regimens include, but are not limited to, 1×10³, 3×10³, 5×10³, 1×10⁴, 3×10⁴, 5×10⁴, 1×10⁵, 3×10⁵, 5×10⁵5×10⁹, or 1×10¹⁰ placental cells or between 1×10³ to 3×10³, 3×10³ to 5×10³, 5×10³ to 1×10⁴, 1×10⁴ to 3×10⁴, 3×10⁴ to 5×10⁴, 5×10⁴ to 1×10⁵, 1×10⁵ to 3×10⁵, 3×10⁵ to 5×10⁵, 5×10⁵ to 1×10⁶, 1×10⁶ to 3×10⁶, 3×10⁶ to 5×10⁶, 5×10⁶ to 1×10⁷, 1×10⁷ to 3×10⁷, 3×10⁷ to 5×10⁷, 5×10⁷ to 1×10⁸, 1×10⁸ to 3×10⁸, 3×10⁸ to 5×10⁸, 5×10⁸ to 1×10⁹, 1×10⁹ to 5×10⁹, or 5×10⁹ to 1×10¹⁰ placental stem cells. In a specific embodiment, the dose of placental stem cells in a pharmaceutical composition is 1×10³ placental stem cells. In another specific embodiment, the dose of placental stem cells in a pharmaceutical composition is 3×10³ placental stem cells. In another specific embodiment, the dose of placental stem cells in a pharmaceutical composition is 3×10⁴ placental stem cells. In another specific embodiment, the dose of placental stem cells in a pharmaceutical composition is 3×10⁵ placental stem cells. In another specific embodiment, the dose of placental stem cells in a pharmaceutical composition is 1×10⁶ placental stem cells. In another specific embodiment, the dose of placental stem cells in a pharmaceutical composition is 3×10⁶ placental stem cells. In another specific embodiment, the dose of placental stem cells in a pharmaceutical composition is 3×10⁷ placental stem cells.

In certain embodiments, a pharmaceutical composition comprising placental stem cells (e.g., CD10+, CD105+, CD200+, CD34− placental stem cells) is administered to a subject having diabetic foot ulcer as a single dose followed by a second dose about 1 month later (e.g., about 27, 28, 29, 30, 31, 32, or 33 days after the initial dose). In certain embodiments, a pharmaceutical composition comprising placental stem cells (e.g., CD10+, CD105+, CD200+, CD34− placental stem cells) is administered to a subject having diabetic foot ulcer as a single dose followed by a second dose about 1 month later and a third dose about one month after that (i.e., about two months after the initial administration, e.g., on or about day 55, 56, 57, 58, 59, 60, 61, 62, 63, or 64 following the initial administration). Doses of placental stem cells administered according to such regimens include, but are not limited to, 1×10³, 3×10³, 5×10³, 1×10⁴, 3×10⁴, 5×10⁴, 1×10⁵, 3×10⁵, 5×10⁵, 1×10⁶, 3×10⁶, 5×10⁶, 1×10⁷, 3×10⁷, 5×10⁷, 1×10⁸, 3×10⁸, 5×10⁸, 1×10⁹, 5×10⁹, or 1×10¹⁰ placental cells or between 1×10³ to 3×10³, 3×10³ to 5×10³, 5×10³ to 1×10⁴, 1×10⁴ to 3×10⁴, 3×10⁴ to 5×10⁴, 5×10⁴ to 1×10⁵, 1×10⁵ to 3×10⁵, 3×10⁵ to 5×10⁵, 5×10⁵ to 1×10⁶, 1×10⁶ to 3×10⁶, 3×10⁶ to 5×10⁶, 5×10⁶ to 1×10⁷, 1×10⁷ to 3×10⁷, 3×10⁷ to 5×10⁷, 5×10⁷ to 1×10⁸, 1×10⁸ to 3×10⁸, 3×10⁸ to 5×10⁸, 5×10⁸ to 1×10⁹, 1×10⁹ to 5×10⁹, or 5×10⁹ to 1×10¹⁰ placental stem cells. In a specific embodiment, the dose of placental stem cells in a pharmaceutical composition is 1×10³ placental stem cells. In another specific embodiment, the dose of placental stem cells in a pharmaceutical composition is 3×10³ placental stem cells. In another specific embodiment, the dose of placental stem cells in a pharmaceutical composition is 3×10⁴ placental stem cells. In another specific embodiment, the dose of placental stem cells in a pharmaceutical composition is 3×10⁵ placental stem cells. In another specific embodiment, the dose of placental stem cells in a pharmaceutical composition is 1×10⁶ placental stem cells. In another specific embodiment, the dose of placental stem cells in a pharmaceutical composition is 3×10⁶ placental stem cells. In another specific embodiment, the dose of placental stem cells in a pharmaceutical composition is 3×10⁷ placental stem cells.

The pharmaceutical compositions provided herein comprise populations of cells that comprise 50% viable cells or more (that is, at least 50% of the cells in the population are functional or living). Preferably, at least 60% of the cells in the population are viable. More preferably, at least 70%, 80%, 90%, 95%, or 99% of the cells in the population in the pharmaceutical composition are viable.

The pharmaceutical compositions provided herein can comprise one or more compounds that, e.g., facilitate engraftment (e.g., anti-T-cell receptor antibodies, an immunosuppressant, or the like); stabilizers such as albumin, dextran 40, gelatin, hydroxyethyl starch, plasmalyte, and the like.

When formulated as an injectable solution, in one embodiment, the pharmaceutical composition comprises about 1% to 1.5% HSA and about 2.5% dextran. In a preferred embodiment, the pharmaceutical composition comprises from about 5×106 cells per milliliter to about 2×10⁷ cells per milliliter in a solution comprising 5% HSA and 10% dextran, optionally comprising an immunosuppressant, e.g., cyclosporine A at, e.g., 10 mg/kg.

In other embodiments, the pharmaceutical composition, e.g., a solution, comprises a plurality of cells, e.g., isolated placental cells, for example, placental stem cells or placental multipotent cells, wherein said pharmaceutical composition comprises between about 1.0±0.3×10⁶ cells per milliliter to about 5.0±1.5×10⁶ cells per milliliter. In other embodiments, the pharmaceutical composition comprises between about 1.5×10⁶ cells per milliliter to about 3.75×10⁶ cells per milliliter. In other embodiments, the pharmaceutical composition comprises between about 1×10⁶ cells/mL to about 50×10⁶ cells/mL, about 1×10⁶ cells/mL to about 40×10⁶ cells/mL, about 1×10⁶ cells/mL to about 30×10⁶ cells/mL, about 1×10⁶ cells/mL to about 20×10⁶ cells/mL, about 1×10⁶ cells/mL to about 15×10⁶ cells/mL, or about 1×10⁶ cells/mL to about 10×10⁶ cells/mL. In certain embodiments, the pharmaceutical composition comprises no visible cell clumps (i.e., no macro cell clumps), or substantially no such visible clumps. As used herein, “macro cell clumps” means an aggregation of cells visible without magnification, e.g., visible to the naked eye, and generally refers to a cell aggregation larger than about 150 microns. In some embodiments, the pharmaceutical composition comprises about 2.5%, 3.0%, 3.5%, 4.0%, 4.5%, 5.0%, 5.5%, 6.0%, 6.5%, 7.0%, 7.5% 8.0%, 8.5%, 9.0%, 9.5% or 10% dextran, e.g., dextran-40. In a specific embodiment, said composition comprises about 7.5% to about 9% dextran-40. In a specific embodiment, said composition comprises about 5.5% dextran-40. In certain embodiments, the pharmaceutical composition comprises from about 1% to about 15% human serum albumin (HSA). In specific embodiments, the pharmaceutical composition comprises about 1%, 2%, 3%, 4%, 5%, 65, 75, 8%, 9%, 10%, 11%, 12%, 13%, 14% or 15% HSA. In a specific embodiment, said cells have been cryopreserved and thawed. In another specific embodiment, said cells have been filtered through a 70 μM to 100 μM filter. In another specific embodiment, said composition comprises no visible cell clumps. In another specific embodiment, said composition comprises fewer than about 200 cell clumps per 106 cells, wherein said cell clumps are visible only under a microscope, e.g., a light microscope. In another specific embodiment, said composition comprises fewer than about 150 cell clumps per 10⁶ cells, wherein said cell clumps are visible only under a microscope, e.g., a light microscope. In another specific embodiment, said composition comprises fewer than about 100 cell clumps per 10⁶ cells, wherein said cell clumps are visible only under a microscope, e.g., a light microscope.

In a specific embodiment, the pharmaceutical composition comprises about 1.0±0.3×10⁶ cells per milliliter, about 5.5% dextran-40 (w/v), about 10% HSA (w/v), and about 5% DMSO (v/v). In another specific embodiment, a pharmaceutical composition comprising placental stem cells provided herein comprises about 5.75% dextran 40, about 10% human serum albumin, and about 2.5% DMSO.

In other embodiments, the pharmaceutical composition comprises a plurality of cells, e.g., a plurality of isolated placental cells in a solution comprising 10% dextran-40, wherein the pharmaceutical composition comprises between about 1.0±0.3×10⁶ cells per milliliter to about 5.0±1.5×10⁶ cells per milliliter, and wherein said composition comprises no cell clumps visible with the unaided eye (i.e., comprises no macro cell clumps). In some embodiments, the pharmaceutical composition comprises between about 1.5×10⁶ cells per milliliter to about 3.75×10⁶ cells per milliliter. In a specific embodiment, said cells have been cryopreserved and thawed. In another specific embodiment, said cells have been filtered through a 70 μM to 100 μM filter. In another specific embodiment, said composition comprises fewer than about 200 micro cell clumps (that is, cell clumps visible only with magnification) per 10⁶ cells. In another specific embodiment, the pharmaceutical composition comprises fewer than about 150 micro cell clumps per 10⁶ cells. In another specific embodiment, the pharmaceutical composition comprises fewer than about 100 micro cell clumps per 10⁶ cells. In another specific embodiment, the pharmaceutical composition comprises less than 15%, 14%, 13%, 12%, 11%, 10%, 9%, 8%, 7%, 6%, 5%, 4%, 3%, or 2% DMSO, or less than 1%, 0.9%, 0.8%, 0.7%, 0.6%, 0.5%, 0.4%, 0.3%, 0.2%, or 0.1% DMSO.

Further provided herein are compositions comprising cells, wherein said compositions are produced by one of the methods disclosed herein. For example, in one embodiment, the pharmaceutical composition comprises cells, wherein the pharmaceutical composition is produced by a method comprising filtering a solution comprising placental cells, e.g., placental stem cells or placental multipotent cells, to form a filtered cell-containing solution; diluting the filtered cell-containing solution with a first solution to about 1 to 50×10⁶, 1 to 40×10⁶, 1 to 30×10⁶, 1 to 20×10⁶, 1 to 15×10⁶, or 1 to 10×10⁶ cells per milliliter, e.g., prior to cryopreservation; and diluting the resulting filtered cell-containing solution with a second solution comprising dextran, but not comprising human serum albumin (HSA) to produce said composition. In certain embodiments, said diluting is to no more than about 15×10⁶ cells per milliliter. In certain embodiments, said diluting is to no more than about 10±3×10⁶ cells per milliliter. In certain embodiments, said diluting is to no more than about 7.5×10⁶ cells per milliliter. In other certain embodiments, if the filtered cell-containing solution, prior to the dilution, comprises less than about 15×10⁶ cells per milliliter, filtration is optional. In other certain embodiments, if the filtered cell-containing solution, prior to the dilution, comprises less than about 10±3×10⁶ cells per milliliter, filtration is optional. In other certain embodiments, if the filtered cell-containing solution, prior to the dilution, comprises less than about 7.5×10⁶ cells per milliliter, filtration is optional.

In a specific embodiment, the cells are cryopreserved between said diluting with a first dilution solution and said diluting with said second dilution solution. In another specific embodiment, the first dilution solution comprises dextran and HSA. The dextran in the first dilution solution or second dilution solution can be dextran of any molecular weight, e.g., dextran having a molecular weight of from about 10 kDa to about 150 kDa. In some embodiments, said dextran in said first dilution solution or said second solution is about 2.5%, 3.0%, 3.5%, 4.0%, 4.5%, 5.0%, 5.5%, 6.0%, 6.5%, 7.0%, 7.5% 8.0%, 8.5%, 9.0%, 9.5% or 10% dextran. In another specific embodiment, the dextran in said first dilution solution or said second dilution solution is dextran-40. In another specific embodiment, the dextran in said first dilution solution and said second dilution solution is dextran-40. In another specific embodiment, said dextran-40 in said first dilution solution is 5.0% dextran-40. In another specific embodiment, said dextran-40 in said first dilution solution is 5.5% dextran-40. In another specific embodiment, said dextran-40 in said second dilution solution is 10% dextran-40. In another specific embodiment, said HSA in said solution comprising HSA is 1 to 15% HSA. In another specific embodiment, said HSA in said solution comprising HSA is about 1%, 2%, 3%, 4%, 5%, 6%, 7%, 8%, 9%, 10%, 11%, 12%, 13%, 14% or 15% HSA. In another specific embodiment, said HSA in said solution comprising HSA is 10% HSA. In another specific embodiment, said first dilution solution comprises HSA. In another specific embodiment, said HSA in said first dilution solution is 10% HSA. In another specific embodiment, said first dilution solution comprises a cryoprotectant. In another specific embodiment, said cryoprotectant is DMSO. In another specific embodiment, said dextran-40 in said second dilution solution is about 10% dextran-40. In another specific embodiment, said composition comprising cells comprises about 7.5% to about 9% dextran. In another specific embodiment, the pharmaceutical composition comprises from about 1.0±0.3×10⁶ cells per milliliter to about 5.0±1.5×10⁶ cells per milliliter. In another specific embodiment, the pharmaceutical composition comprises from about 1.5×10⁶ cells per milliliter to about 3.75×10⁶ cells per milliliter.

In another embodiment, the pharmaceutical composition is made by a method comprising (a) filtering a cell-containing solution comprising placental cells, e.g., placental stem cells or placental multipotent cells, prior to cryopreservation to produce a filtered cell-containing solution; (b) cryopreserving the cells in the filtered cell-containing solution at about 1 to 50×10⁶, 1 to 40×10⁶, 1 to 30×10⁶, 1 to 20×10⁶, 1 to 15×10⁶, or 1 to 10×10⁶ cells per milliliter; (c) thawing the cells; and (d) diluting the filtered cell-containing solution about 1:1 to about 1:11 (v/v) with a dextran-40 solution. In certain embodiments, if the number of cells is less than about 10±3×10⁶ cells per milliliter prior to step (a), filtration is optional. In another specific embodiment, the cells in step (b) are cryopreserved at about 10±3×10⁶ cells per milliliter. In another specific embodiment, the cells in step (b) are cryopreserved in a solution comprising about 5% to about 10% dextran-40 and HSA. In certain embodiments, said diluting in step (b) is to no more than about 15×10⁶ cells per milliliter.

In another embodiment, the pharmaceutical composition is made by a method comprising: (a) suspending placental cells, e.g., placental stem cells or placental multipotent cells, in a 5.5% dextran-40 solution that comprises 10% HSA to form a cell-containing solution; (b) filtering the cell-containing solution through a 70 μM filter; (c) diluting the cell-containing solution with a solution comprising 5.5% dextran-40, 10% HSA, and 5% DMSO to about 1 to 50×10⁶, 1 to 40×10⁶, 1 to 30×10⁶, 1 to 20×10⁶, 1 to 15×10⁶, or 1 to 10×10⁶ cells per milliliter; (d) cryopreserving the cells; (e) thawing the cells; and (f) diluting the cell-containing solution 1:1 to 1:11 (v/v) with 10% dextran-40. In certain embodiments, said diluting in step (c) is to no more than about 15×10⁶ cells per milliliter. In certain embodiments, said diluting in step (c) is to no more than about 10±3×10⁶ cells/mL. In certain embodiments, said diluting in step (c) is to no more than about 7.5×10⁶ cells/mL.

In another embodiment, the composition comprising cells is made by a method comprising: (a) centrifuging a plurality of cells to collect the cells; (b) resuspending the cells in 5.5% dextran-40; (c) centrifuging the cells to collect the cells; (d) resuspending the cells in a 5.5% dextran-40 solution that comprises 10% HSA; (e) filtering the cells through a 70 μM filter; (f) diluting the cells in 5.5% dextran-40, 10% HSA, and 5% DMSO to about 1 to 50×10⁶, 1 to 40×10⁶, 1 to 30×10⁶, 1 to 20×10⁶, 1 to 15×10⁶, or 1 to 10×10⁶ cells per milliliter; (g) cryopreserving the cells; (h) thawing the cells; and (i) diluting the cells 1:1 to 1:11 (v/v) with 10% dextran-40. In certain embodiments, said diluting in step (f) is to no more than about 15×10⁶ cells per milliliter. In certain embodiments, said diluting in step (f) is to no more than about 10±3×10⁶ cells/mL. In certain embodiments, said diluting in step (f) is to no more than about 7.5×10⁶ cells/mL. In other certain embodiments, if the number of cells is less than about 10±3×10⁶ cells per milliliter, filtration is optional.

The compositions, e.g., pharmaceutical compositions comprising the isolated placental cells, described herein can comprise any of the isolated placental cells described herein.

Other injectable formulations, suitable for the administration of cellular products, may be used.

In one embodiment, the pharmaceutical composition comprises isolated placental cells that are substantially, or completely, non-maternal in origin, that is, have the fetal genotype; e.g., at least about 90%, 95%, 98%, 99% or about 100% are non-maternal in origin. For example, in one embodiment a pharmaceutical composition comprises a population of isolated placental cells that are CD200+ and HLA-G; CD73+, CD105+, and CD200+; CD200+ and OCT-4+; CD73+, CD105+ and HLA-G; CD73+ and CD105+ and facilitate the formation of one or more embryoid-like bodies in a population of placental cells comprising said population of isolated placental cell when said population of placental cells is cultured under conditions that allow the formation of an embryoid-like body; or OCT-4+ and facilitate the formation of one or more embryoid-like bodies in a population of placental cells comprising said population of isolated placental cell when said population of placental cells is cultured under conditions that allow the formation of an embryoid-like body; or a combination of the foregoing, wherein at least 70%, 80%, 90%, 95% or 99% of said isolated placental cells are non-maternal in origin. In another embodiment, a pharmaceutical composition comprises a population of isolated placental cells that are CD10+, CD105+ and CD34; CD10+, CD105+, CD200+ and CD34; CD10+, CD105+, CD200+, CD34 and at least one of CD90+ or CD45−; CD10+, CD90+, CD105+, CD200+, CD34 and CD45−; CD10+, CD90+, CD105+, CD200+, CD34 and CD45−; CD200+ and HLA-G; CD73+, CD105+, and CD200+; CD200+ and OCT-4+; CD73+, CD105+ and HLA-G; CD73+ and CD105+ and facilitate the formation of one or more embryoid-like bodies in a population of placental cells comprising said isolated placental cells when said population of placental cells is cultured under conditions that allow the formation of an embryoid-like body; OCT-4+ and facilitate the formation of one or more embryoid-like bodies in a population of placental cells comprising said isolated placental cells when said population of placental cells is cultured under conditions that allow the formation of an embryoid-like body; or one or more of CD117, CD133, KDR, CD80, CD86, HLA-A,B,C+, HLA-DP,DQ,DR and/or PDL1+; or a combination of the foregoing, wherein at least 70%, 80%, 90%, 95% or 99% of said isolated placental cells are non-maternal in origin. In a specific embodiment, the pharmaceutical composition additionally comprises a stem cell that is not obtained from a placenta.

Isolated placental cells in the compositions, e.g., pharmaceutical compositions, provided herein, can comprise placental cells derived from a single donor, or from multiple donors. The isolated placental cells can be completely HLA-matched to an intended recipient, or partially or completely HLA-mismatched.

5.3.3 Matrices Comprising Isolated Placental Cells

Further provided herein are compositions comprising matrices, hydrogels, scaffolds, and the like that comprise a placental cell, or a population of isolated placental cells. Such compositions can be used in the place of, or in addition to, cells in liquid suspension.

The isolated placental cells described herein can be seeded onto a natural matrix, e.g., a placental biomaterial such as an amniotic membrane material. Such an amniotic membrane material can be, e.g., amniotic membrane dissected directly from a mammalian placenta; fixed or heat-treated amniotic membrane, substantially dry (i.e., <20% H2O) amniotic membrane, chorionic membrane, substantially dry chorionic membrane, substantially dry amniotic and chorionic membrane, and the like. Preferred placental biomaterials on which isolated placental cells can be seeded are described in Hariri, U.S. Application Publication No. 2004/0048796, the disclosure of which is incorporated herein by reference in its entirety.

The isolated placental cells described herein can be suspended in a hydrogel solution suitable for, e.g., injection. Suitable hydrogels for such compositions include self-assembling peptides, such as RAD16. In one embodiment, a hydrogel solution comprising the cells can be allowed to harden, for instance in a mold, to form a matrix having cells dispersed therein for implantation. Isolated placental cells in such a matrix can also be cultured so that the cells are mitotically expanded prior to implantation. The hydrogel is, e.g., an organic polymer (natural or synthetic) that is cross-linked via covalent, ionic, or hydrogen bonds to create a three-dimensional open-lattice structure that entraps water molecules to form a gel. Hydrogel-forming materials include polysaccharides such as alginate and salts thereof, peptides, polyphosphazines, and polyacrylates, which are crosslinked ionically, or block polymers such as polyethylene oxide-polypropylene glycol block copolymers which are crosslinked by temperature or pH, respectively. In some embodiments, the hydrogel or matrix is biodegradable.

In some embodiments, the formulation comprises an in situ polymerizable gel (see., e.g., U.S. Patent Application Publication 2002/0022676, the disclosure of which is incorporated herein by reference in its entirety; Anseth et al., J. Control Release, 78(1-3):199-209 (2002); Wang et al., Biomaterials, 24(22):3969-80 (2003).

In some embodiments, the polymers are at least partially soluble in aqueous solutions, such as water, buffered salt solutions, or aqueous alcohol solutions, that have charged side groups, or a monovalent ionic salt thereof. Examples of polymers having acidic side groups that can be reacted with cations are poly(phosphazenes), poly(acrylic acids), poly(methacrylic acids), copolymers of acrylic acid and methacrylic acid, poly(vinyl acetate), and sulfonated polymers, such as sulfonated polystyrene. Copolymers having acidic side groups formed by reaction of acrylic or methacrylic acid and vinyl ether monomers or polymers can also be used. Examples of acidic groups are carboxylic acid groups, sulfonic acid groups, halogenated (preferably fluorinated) alcohol groups, phenolic OH groups, and acidic OH groups.

In a specific embodiment, the matrix is a felt, which can be composed of a multifilament yarn made from a bioabsorbable material, e.g., PGA, PLA, PCL copolymers or blends, or hyaluronic acid. The yarn is made into a felt using standard textile processing techniques consisting of crimping, cutting, carding and needling. In another preferred embodiment the cells of the invention are seeded onto foam scaffolds that may be composite structures. In addition, the three-dimensional framework may be molded into a useful shape, such as a specific structure in the body to be repaired, replaced, or augmented. Other examples of scaffolds that can be used include nonwoven mats, porous foams, or self assembling peptides. Nonwoven mats can be formed using fibers comprised of a synthetic absorbable copolymer of glycolic and lactic acids (e.g., PGA/PLA) (VICRYL, Ethicon, Inc., Somerville, N.J.). Foams, composed of, e.g., poly(8-caprolactone)/poly(glycolic acid) (PCL/PGA) copolymer, formed by processes such as freeze-drying, or lyophilization (see, e.g., U.S. Pat. No. 6,355,699), can also be used as scaffolds.

The isolated placental cells described herein or co-cultures thereof can be seeded onto a three-dimensional framework or scaffold and implanted in vivo. Such a framework can be implanted in combination with any one or more growth factors, cells, drugs or other components that, e.g., stimulate tissue formation.

Examples of scaffolds that can be used include nonwoven mats, porous foams, or self assembling peptides. Nonwoven mats can be formed using fibers comprised of a synthetic absorbable copolymer of glycolic and lactic acids (e.g., PGA/PLA) (VICRYL, Ethicon, Inc., Somerville, N.J.). Foams, composed of, e.g., poly(8-caprolactone)/poly(glycolic acid) (PCL/PGA) copolymer, formed by processes such as freeze-drying, or lyophilization (see, e.g., U.S. Pat. No. 6,355,699), can also be used as scaffolds.

In another embodiment, isolated placental cells can be seeded onto, or contacted with, a felt, which can be, e.g., composed of a multifilament yarn made from a bioabsorbable material such as PGA, PLA, PCL copolymers or blends, or hyaluronic acid.

The isolated placental cells provided herein can, in another embodiment, be seeded onto foam scaffolds that may be composite structures. Such foam scaffolds can be molded into a useful shape, such as that of a portion of a specific structure in the body to be repaired, replaced or augmented. In some embodiments, the framework is treated, e.g., with 0.1M acetic acid followed by incubation in polylysine, PBS, and/or collagen, prior to inoculation of the cells in order to enhance cell attachment. External surfaces of a matrix may be modified to improve the attachment or growth of cells and differentiation of tissue, such as by plasma-coating the matrix, or addition of one or more proteins (e.g., collagens, elastic fibers, reticular fibers), glycoproteins, glycosaminoglycans (e.g., heparin sulfate, chondroitin-4-sulfate, chondroitin-6-sulfate, dermatan sulfate, keratin sulfate, etc.), a cellular matrix, and/or other materials such as, but not limited to, gelatin, alginates, agar, agarose, and plant gums, and the like.

In some embodiments, the scaffold comprises, or is treated with, materials that render it non-thrombogenic. These treatments and materials may also promote and sustain endothelial growth, migration, and extracellular matrix deposition. Examples of these materials and treatments include but are not limited to natural materials such as basement membrane proteins such as laminin and Type IV collagen, synthetic materials such as EPTFE, and segmented polyurethaneurea silicones, such as PURSPAN™ (The Polymer Technology Group, Inc., Berkeley, Calif.). The scaffold can also comprise anti-thrombotic agents such as heparin; the scaffolds can also be treated to alter the surface charge (e.g., coating with plasma) prior to seeding with isolated placental cells.

The placental cells (e.g., PDACs) provided herein can also be seeded onto, or contacted with, a physiologically-acceptable ceramic material including, but not limited to, mono-, di-, tri-, alpha-tri-, beta-tri-, and tetra-calcium phosphate, hydroxyapatite, fluoroapatites, calcium sulfates, calcium fluorides, calcium oxides, calcium carbonates, magnesium calcium phosphates, biologically active glasses such as BIOGLASS®, and mixtures thereof. Porous biocompatible ceramic materials currently commercially available include SURGIBONE® (CanMedica Corp., Canada), ENDOBON® (Merck Biomaterial France, France), CEROS® (Mathys, AG, Bettlach, Switzerland), and mineralized collagen bone grafting products such as HEALOS™ (DePuy, Inc., Raynham, Mass.) and VITOSS®, RHAKOSS™, and CORTOSS® (Orthovita, Malvern, Pa.). The framework can be a mixture, blend or composite of natural and/or synthetic materials.

In one embodiment, the isolated placental cells are seeded onto, or contacted with, a suitable scaffold at about 0.5×10⁶ to about 8×10⁶ cells/mL.

### 6. EXAMPLES

### 6.1 Example 1: Phenotypic Characterization of Placental Derived Adherent Cells

This example demonstrates secretion of angiogenic factors by placental cells (CD10⁺, CD34⁻, CD105⁺, CD200⁺ placental stem cells, also called PDACs).

6.1.1 Secretome Profiling for Evaluation of Angiogenic Potency of Placental Derived Adherent Cells

MulitplexBead Assay:

Placental derived adherent cells at passage 6 were plated at equal cell numbers in growth medium and conditioned media were collected after 48 hours. Simultaneous qualitative analysis of multiple angiogenic cytokines/growth factors in cell-conditioned media was performed using magnetic bead-based multiplex assays (Bio-Plex Pro™, Bio-Rad, CA) assays are that allow the measurement of angiogenic biomarkers in diverse matrices including serum, plasma, and cell/tissue culture supernatants. The principle of these 96-well plate-formatted, bead-based assays is similar to a capture sandwich immunoassay. An antibody directed against the desired angiogenesis target is covalently coupled to internally dyed beads. The coupled beads are allowed to react with a sample containing the angiogenesis target. After a series of washes to remove unbound protein, a biotinylated detection antibody specific for a different epitope is added to the reaction. The result is the formation of a sandwich of antibodies around the angiogenesis target. Streptavidin-PE is then added to bind to the biotinylated detection antibodies on the bead surface. In brief, Multiplex assays were performed according to manufacturer's instructions and the amount of the respective angiogenic growth factors in the conditioned media was evaluated.

ELISAs:

Quantitative analysis of single angiogenic cytokines/growth factors in cell-conditioned media was performed using commercially available kits from R&D Systems (Minneapolis, Minn.). In brief, ELISA assays were performed according to manufacturer's instructions and the amount of the respective angiogenic growth factors in the conditioned media was evaluated.

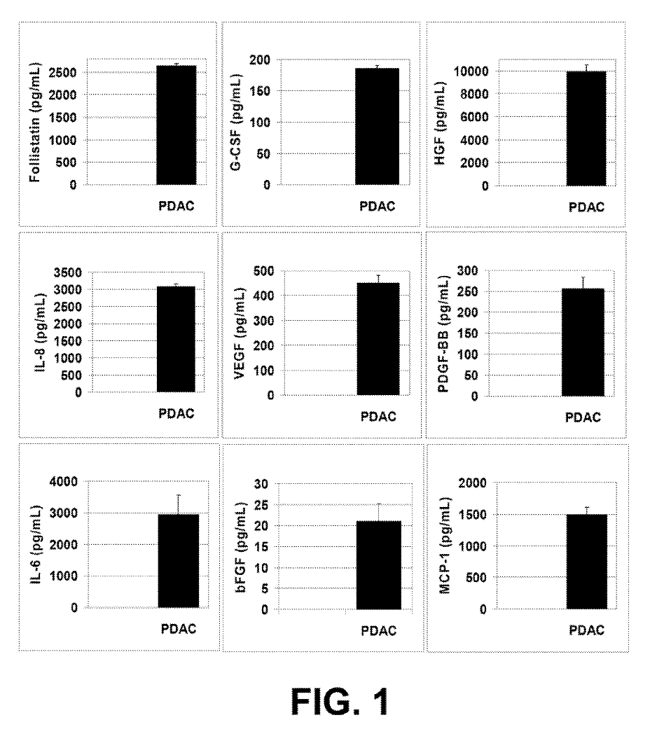

The level of secretion of various angiogenic proteins by PDAC is shown in FIG. 1.

TABLE 1
Multiplex and ELISA results for angiogenic markers

| | | | Secretome |
|---|---|---|---|
| | | | Analysis ELISA, |
| PDAC Marker | Positive | Negative | Multiplex |
| ANG | X | | X |
| EGF | X | | X |
| ENA-78 | X | | X |
| FGF2 | X | | X |
| Follistatin | X | | X |
| G-CSF | X | | X |
| GRO | X | | X |
| HGF | X | | X |
| IL-6 | X | | X |
| IL-8 | X | | X |
| Leptin | X | | X |
| MCP-1 | X | | X |
| MCP-3 | X | | X |
| PDGFB | X | | X |
| PLGF | X | | X |
| Rantes | X | | X |
| TGFB1 | X | | X |
| Thrombopoietin | X | | X |
| TIMP1 | X | | X |
| TIMP2 | X | | X |
| uPAR | X | | X |
| VEGF | X | | X |
| VEGFD | X | | X |

In a separate experiment, PDACs were confirmed to also secrete angiopoietin-1, angiopoietin-2, PECAM-1 (CD31; platelet endothelial cell adhesion molecule), laminin and fibronectin.

### 6.2 Example 2: Functional Characterization of Placental Cells

This Example demonstrates different characteristics of placental cells (CD10⁺, CD34⁻, CD105⁺, CD200⁺ placental stem cells, also called PDACs) associated with angiogenesis and differentiation capability.

6.2.1 HUVEC Tube Formation for Evaluation of Angiogenic Potency of PDACs

Human Umbilical Vein Endothelial Cells (HUVEC) were subcultured at passage 3 or less in EGM-2 medium (Cambrex, East Rutherford, N.J.) for 3 days, and harvested at a confluency of approximately 70%-80%. HUVEC were washed once with basal medium/antibiotics (DMEM/F12 (Gibco)) and resuspended in the same medium at the desired concentration. HUVEC were used within 1 hour of preparation. Human placental collagen (HPC) was brought to a concentration of 1.5 mg/mL in 10 mM HCl (pH 2.25), was neutralized with buffer to pH 7.2, and kept on ice until used. The HPC was combined with the HUVEC suspension at a final cell concentration of 4000 cells/μ1. The resulting HUVEC/HPC suspension was immediately pipetted into 96-well plates at 3 μl per well (plate perimeter must be pre-filled with sterile PBS to avoid evaporation, n=5 per condition). HUVEC drops were incubated at 37° C. and 5% CO₂ for 75-90 minutes without medium addition to allow for collagen polymerization. Upon completion of “dry” incubation, each well was gently filled with 200 μl of conditioned PDAC medium (n=2 cell lines) or control medium (e.g., DMEM/F12 as the negative control, and EGM-2 as the positive control) and incubated at 37° C. and 5% CO₂ for 20 hrs. Conditioned medium was prepared by incubating PDACs at passage 6 in growth medium for 4-6 hours; after attachment and spreading, the medium was changed to DMEM/F12 for 24 hours. After incubation, the medium was removed from the wells without disturbing the HUVEC drops and the wells were washed once with PBS. The HUVEC drops were then fixed for 10 seconds and stained for 1 minute using a Diff-Quik cell staining kit and subsequently rinsed 3× times with sterile water. The stained drops were allowed to air dry and images of each well were acquired using the Zeiss SteReo Discovery V8 microscope. The images were then analyzed using the computer software package ImageJ and/or MatLab. Images were converted from color to 8-bit grayscale images and thresholded to convert to a black and white image. The image was then analyzed using the particle analysis features, which provided pixel density data, including count (number of individual particles), total area, average size (of individual particles), and area fraction, which equates to the amount endothelial tube formation in the assay.

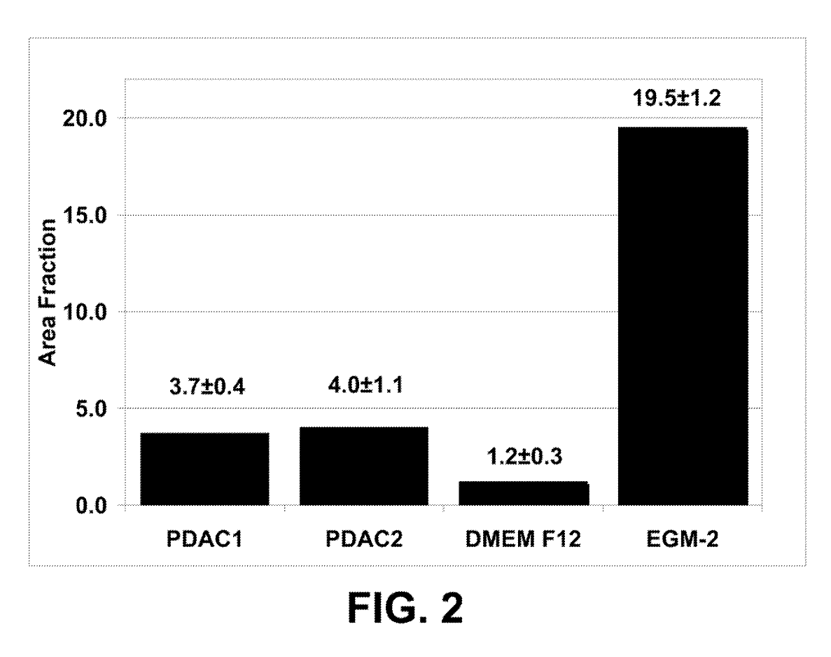

The conditioned medium exerted an angiogenic effect on endothelial cells, as demonstrated by the induction of tube formation (see FIG. 2).

6.2.2 HUVEC Migration Assay

This experiment demonstrated the angiogenic capacity of placental derived adherent cells. HUVECs were grown to monolayer confluence in a fibronectin (FN)-coated 12-well plate and the monolayer was “wounded” with a 1 mL plastic pipette tip to create an acellular line across the well. HUVEC migration was tested by incubating the “wounded” cells with serum-free conditioned medium (EBM2; Cambrex) obtained from PDACs after 3 days of growth. EBM2 medium without cells was used as the control. After 15 hours, the cell migration into the acellular area was recorded (n=3) using an inverted microscope. The pictures were then analyzed using the computer software package ImageJ and/or MatLab. Images were converted from color to 8-bit grayscale images and thresholded to convert to a black and white image. The image was then analyzed using the particle analysis features, which provided pixel density data, including count (number of individual particles), total area, average size (of individual particles), and area fraction, which equates to the amount endothelial migration in the assay. The degree of cell migration was scored against the size of the initially recorded wound line and the results were normalized to 1×10⁶ cells.

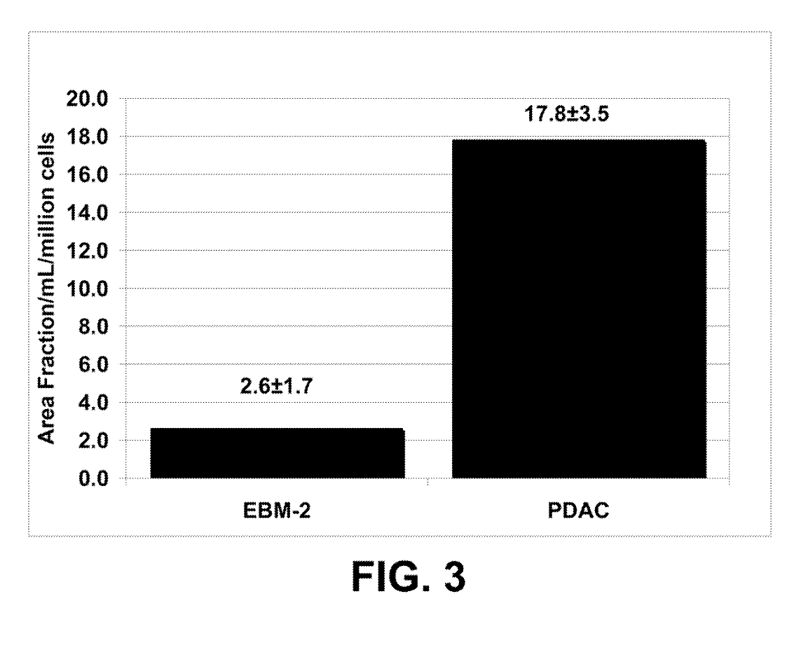

The trophic factors secreted by placental derived adherent cells exerted angiogenic effects on endothelial cells, as demonstrated by the induction of cell migration (FIG. 3).

In a separate experiment, HUVECs were cultured in the bottom of 24 well-plates for overnight establishment in EGM2, followed by a half-day starvation in EBM. Concurrently, media-cultured PDAC were thawed and cultured in transwells (8 μM) overnight. After the EC starvation, the conditioned serum-free DMEM, along with the transwell, was transferred over to the ECs for overnight proliferation. 4 replicates were included in each experiment, and proliferation after 24 hrs was assessed with Promega's Cell Titer Glo Assay. EBM-2 medium was used as the negative control, and EGM-2 was used as the positive control. Error bars denote standard deviations of analytical replicates (n=3).

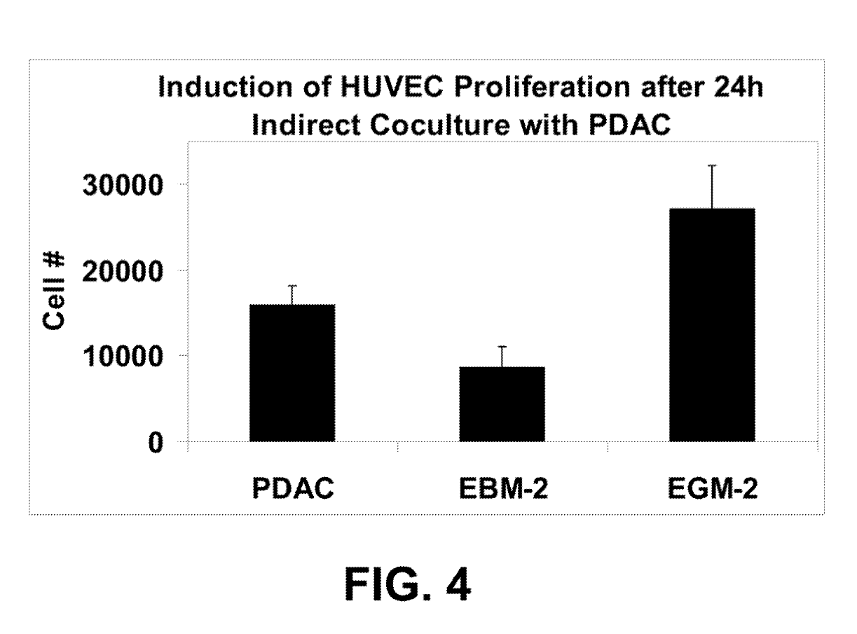

The trophic factors secreted by PDACs resulted in an increase in HUVEC cell number, which is indicative of HUVEC proliferation. See FIG. 4.

6.2.3 Tube Formation for Evaluation of Angiogenic Potency of Placental Derived Adherent Cells

PDACs were grown either in growth medium without VEGF or EGM2-MV with VEGF to evaluate the angiogenic potency of the cells in general, as well as the effect of VEGF on the differentiation potential of the cells. HUVECs, as control cells for tube formation, were grown in EGM2-MV. The cells were cultured in the respective media for 4 to 7 days until they reached 70-80% confluence. Cold (4° C.) MATRIGEL™ solution (50 μL; BD Biosciences) was dispensed into wells of a 12-well plate and the plate was incubated for 60 min at 37° C. to allow the solution to gel. The PDAC and HUVEC cells were trypsinized, resuspended in the appropriate media (with and without VEGF) and 100 μl of diluted cells (1 to 3×10⁴ cells) were added to each of the MATRIGEL™-containing wells. The cells on the polymerized MATRIGEL™, in the presence or absence of 0.5 to 100 ng VEGF, were placed for 4 to 24 hours in a 5% CO2 incubator at 37° C. After incubation the cells were evaluated for signs of tube formation using standard light microscopy.

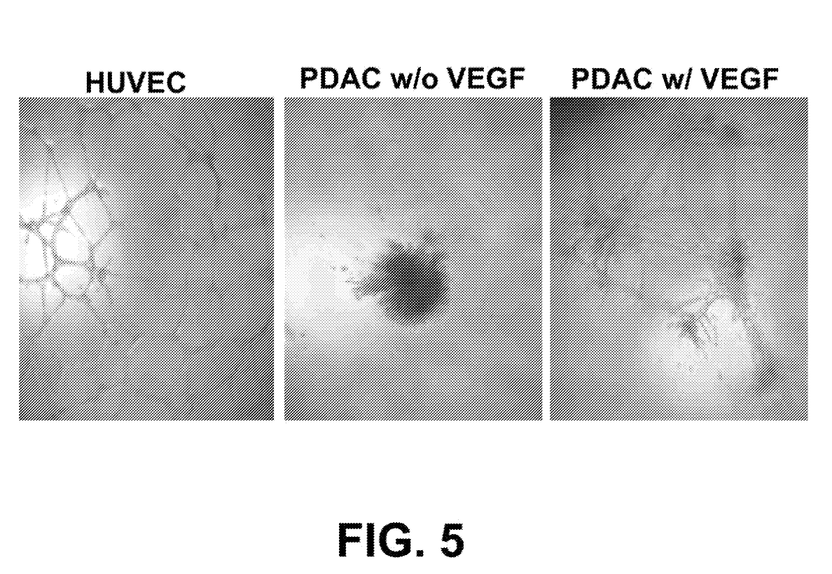

PDACs displayed minimal tube formation in the absence of VEGF, but were induced/differentiated to form tube-like structures through stimulation with VEGF. See FIG. 5.

6.2.4 Hypoxia Responsiveness for Evaluation of Angiogenic Potency of Placental Derived Adherent Cells

To evaluate the angiogenic functionality of endothelial cells and/or endothelial progenitors, cells can be assessed in regard to their capability to secrete angiogenic growth factors under hypoxic and normoxic conditions. Culture under hypoxic conditions usually induces an increased secretion of angiogenic growth factors by either endothelial cells or endothelial progenitor cells, which can be measured in the conditioned media. Placental derived adherent cells were plated at equal cell numbers in their standard growth medium and grown to approximately 70-80% confluence. Subsequently, the cells were switched to serum-free medium (EBM-2) and incubated under normoxic (21% O2) or hypoxic (1% O2) conditions for 24 h. The conditioned media were collected and the secretion of angiogenic growth factors was analyzed using commercially available ELISA kits from R&D Systems. The ELISA assays were performed according to manufacturer's instructions and the amount of the respective angiogenic growth factors (VEGF and IL-8) in the conditioned media was normalized to 1×10⁶ cells.

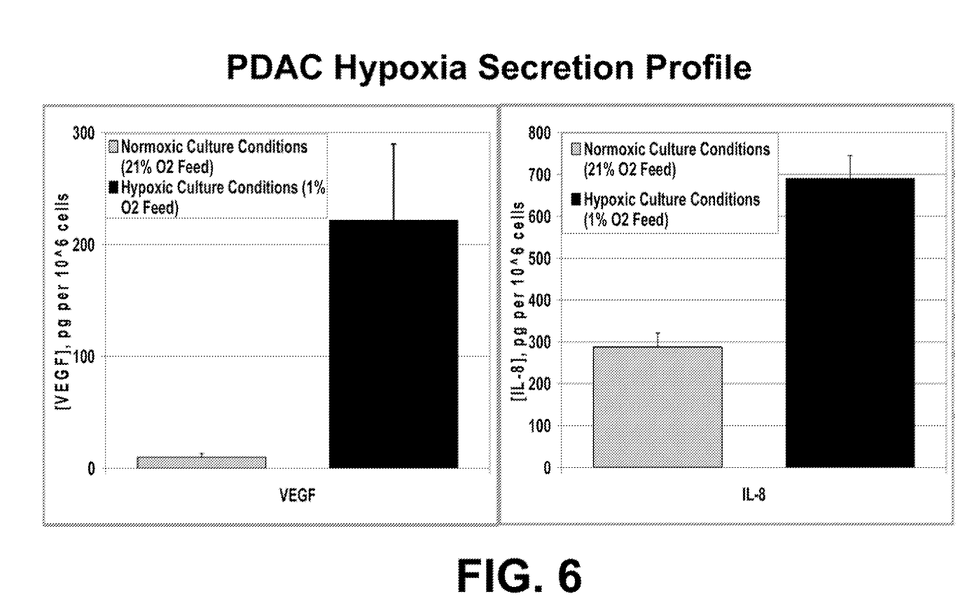

Placental derived adherent cells displayed elevated secretion of various angiogenic growth factors under hypoxic conditions. See FIG. 6.

6.2.5 HUVEC Response to PDAC-Conditioned Medium

PDACs were cultured for 48 hours in growth medium containing 60% DMEM-LG (Gibco); 40% MCBD-201 (Sigma); 2% FBS (Hyclone Labs), 1× insulin-transferrin-selenium (ITS); 10 ng/mL linoleic acid-bovine serum albumin (LA-BSA); 1 n-dexamethasone (Sigma); 100 μM ascorbic acid 2-phosphate (Sigma); 10 ng/mL epidermal growth factor (R & D Systems); and 10 ng/mL platelet-derived growth factor (PDGF-BB) (R & D Systems), and then cultured for an additional 48 hrs in serum-free media. Conditioned medium from PDAC culture was collected and used to stimulate serum-starved HUVECs for 5, 15, and 30 minutes. The HUVECs were subsequently lysed and stained with a BD™ CBA (Cytometric Bead Assay) Cell Signaling Flex Kit (BD Biosciences) for phosphoproteins known to play a role in angiogenic pathway signaling. PDACs were found to be strong activators of AKT-1 (which inhibits apoptotic processes), AKT-2 (which is an important signaling protein in the insulin signaling pathway, and ERK 1/2 cell proliferation pathways in HUVECs. These results further demonstrate the angiogenic capability of PDACs.

### 6.3 Example 3: Induction of Angiogenesis by PDACS

This Example demonstrates that PDACs, as described in Example 1, above, promote angiogenesis in an in vivo assay using chick chorioallantoic membrane (CAM).

Two separate CAM assays were conducted. In the first CAM assay, intact cell pellets from different preparations of PDAC were evaluated. In the second CAM assay, supernatants of different PDAC preparations were evaluated. Fibroblast growth factor (bFGF) was used as a positive control, and MDA-MB-231 human breast cancer cells as a reference, vehicle and medium controls were used as negative controls. The endpoint of the study was to determine the blood vessel densities of all treatment and control groups.

6.3.1 CAM Assay Using PDAC

PDACs, prepared as described above and cryopreserved, were used. PDACs were thawed for dosing and the number of cells dosed on the CAM was determined.

Study Design: The study included 5 groups with 10 embryos in each group. The design of the study is described in Table 2.

TABLE 2
Study groups, chick chorioallantoic membrane angiogenesis assay.

| Group | # of | | |
|---|---|---|---|
| No. | Embryos | Treatment | End Point |
| 1 | 10 | Vehicle control (40 μl of PBS/ | Blood vessel density |
| | | MATRIGEL ™ mixture, 1:1 by volume) | score |
| 2 | 10 | Positive control, treated with bFGF (100 ng/ | Same as group 1 |
| | | CAM in 40 μl of DMEM/ | |
| | | MATRIGEL ™ mixture, 1:1) | |
| 3 | 10 | Medium control (40 μl of DMEM) | Same as group 1 |
| 4 | 10 | PDAC | Same as group 1 |
| 5 | 10 | MDA-MB-231 cells P34, Lot No. 092608 | Same as group 1 |

CAM Assay Procedure: Fresh fertile eggs were incubated for 3 days in a standard egg incubator at 37° C. for 3 days. On Day 3, eggs were cracked under sterile conditions and embryos were placed into twenty 100 mm plastic plates and cultivated at 37° C. in an embryo incubator with a water reservoir on the bottom shelf. Air was continuously bubbled into the water reservoir using a small pump so that the humidity in the incubator was kept constant. On Day 6, a sterile silicon “0” ring was placed on each CAM, and then PDAC at a density of 7.69×105 cells/40 μL of medium/MATRIGEL™ mixture (1:1) were delivered into each “O” ring in a sterile hood. Tables 2A and 2B represent the number of cells used and the amount of medium added to each cell preparation for dosing. Vehicle control embryos received 40 μL of vehicle (PBS/MATRIGEL™, 1:1), positive controls received 100 ng/ml bFGF in 40 μl of DMEM medium/MATRIGEL™ mixture (1:1), and medium controls received 40 μl of DMEM medium alone. Embryos were returned to the incubator after each dosing was completed. On Day 8, embryos were removed from the incubator and kept at room temperature while blood vessel density was determined under each “O” ring using an image capturing system at a magnification of 100×.

Blood vessel density was measured by an angiogenesis scoring system that used arithmetic numbers 0 to 5, or exponential numbers 1 to 32, to indicate the number of blood vessels present at the treatment sites on the CAM. Higher scoring numbers represented higher vessel density, while 0 represented no angiogenesis. The percent of inhibition at each dosing site was calculated using the score recorded for that site divided by the mean score obtained from control samples for each individual experiment. The percent of inhibition for each dose of a given compound was calculated by pooling all results obtained for that dose from 8-10 embryos.

TABLE 3
Amount of medium added to each cell preparation for
normalization of the final cell suspension for dosing

| | | | Final Volume |
|---|---|---|---|
| | Pellet | Normalization with DMEM | of Cell |
| Cell Line | size | and MATRIGEL ™ | Suspension |
| PDAC | 260 μL | 0 μL + 260 μL MATRIGEL ™ | 520 μL |
| MDA- | 40 μL | 220 μL + 260 μL MATRIGEL ™ | 520 μL |
| MB-231 | | | |

PDAC were used at Passage 6.

### Results

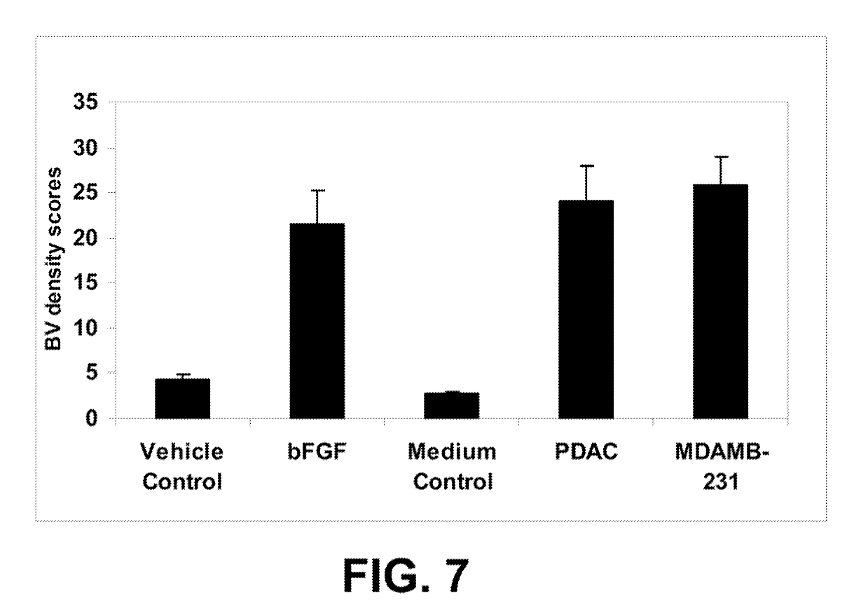

The results of blood vessel density scores are presented in FIG. 7. The results clearly indicate that the blood vessel density scores of chick chorioallantoic membranes treated with PDAC cell suspensions, or 100 ng/mL of bFGF, or MDAMB231 breast cancer cell suspensions were statistically significantly higher compared to those of the vehicle control CAMs (P<0.001, Student's “t” test). The medium used for culturing PDACs (negative control) did not have any effect on the blood vessel density. Further, the induction of blood vessel density of PDAC preparations showed some variation, but the variations were not statistically significant.

6.3.2 CAM Assay Using PDAC Supernatants

Supernatant samples from MDA-MB-231 cells and PDAC were used in a second CAM assay as described above. bFGF and MDA-MB-231 supernatants were used as positive controls, medium and vehicle were used as negative controls.

Study Design: The study included 5 groups with 10 embryos in each group. The design of the study is described in Table 4.

TABLE 4
Study Design - CAM assay using cell supernatants

| Group | # of | | |
|---|---|---|---|
| No. | Embryos | Treatment | End Point |
| 1 | 10 | Vehicle control (40 μl of | Blood vessel density |
| | | PBS/MATRIGEL ™ mixture, | score |
| | | 1:1 by volume) | |
| 2 | 10 | Positive control, treated with | Same as group 1 |
| | | bFGF (100 ng/CAM in 40 μl | |
| | | of DMEM/MATRIGEL ™ | |
| | | mixture, 1:1) | |
| 3 | 10 | Medium control (40 μl of | Same as group 1 |
| | | DMEM) | |
| 4 | 10 | Supernatant of PDAC | Same as group 1 |
| 5 | 10 | Supernatant of MDAMB231 | Same as group 1 |
| | | cells (P34) | |

PDAC supernatants were obtained from cells at Passage 6.

CAM Assay Procedure: The assay procedure was the same as described in section 6.3.1, above. The only difference was that supernatant from each stem cell preparation or from MDA-MB-231 cells was used as test material. For dosing, each supernatant was mixed with MATRIGEL™ (1:1 by volume) and 40 μL of the mixture was dosed to each embryo.

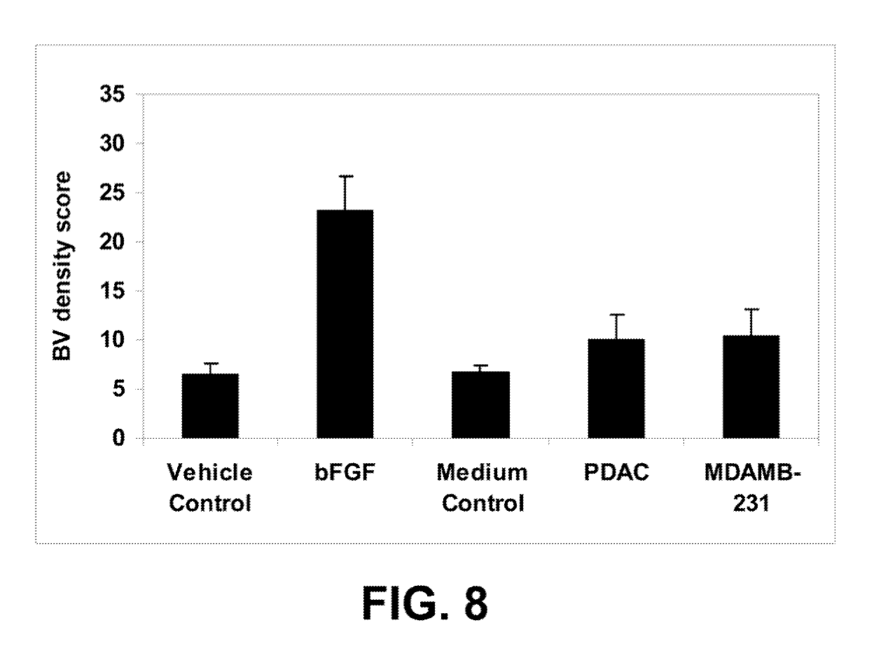

Results: Blood vessel density scores (see FIG. 8) indicate that the induction of blood vessel formation by the supernatant of each stem cell preparation differed. Supernatant samples from PDAC showed significant effect on blood vessel induction with P<0.01, P<0.001, and P<0.02 (Student's “t” test) respectively. As expected, positive control bFGF also showed potent induction of blood vessel formation as seen above in CAM assay no. 1 (P<0.001, Student's “t” test). However, supernatant from MDA-MB-231 human breast cancer cells did not show significant induction on blood vessel formation compared to the vehicle controls. As previously shown, culture medium alone did not have any effect.

### 6.4 Example 4: PDAC Exhibit Neuroprotective Effect

This Example demonstrates that PDAC have a neuroprotective effect in low-oxygen and low-glucose conditions using an oxygen-glucose deprivation (OGD) insult assay, and reduce reactive oxygen species. As such, these results indicate that PDAC would be useful in treating ischemic conditions such as stroke or peripheral vascular disease.

Human neurons (ScienCell, catalog #1520) were cultured as per manufacturer's recommendations. Briefly, culture vessels were coated with Poly-L-Lysine (2 μg/mL) in sterile distilled water for 1 hour at 37° C. The vessel was washed with double distilled H₂O three times. Neuron Medium (ScienCell) was added to vessel and equilibrated to 37° C. in an incubator. Neurons were thawed, and added directly into the vessels without centrifugation. During subsequent culture, medium was changed the day following culture initiation, and every other day thereafter. The neurons were typically ready for insult by day 4.

OGD medium (Dulbecco's Modified Eagle's Medium-Glucose Free) was prepared by first warming the medium in a water bath, in part to reduce the solubility of oxygen in the liquid medium. 100% nitrogen was bubbled for 30 minutes through the medium using a 0.5 μm diffusing stone to remove dissolved oxygen. HEPES buffer was added to a final concentration of 1 mM. Medium was added directly to the neurons at the end of the sparge. A small sample of the medium was aliquoted for confirmation of oxygen levels using a dip-type oxygen sensor. Oxygen levels were typically reduced to 0.9% to about 5.0% oxygen.

A hypoxia chamber was prepared by placing the chamber in an incubator at 37° C. for at least 4 hours (overnight preferred) prior to gassing. Medium in the culture vessels was removed and replaced with de-gassed medium, and the culture vessels were placed in the hypoxia chamber. The hypoxia chamber was then flushed with 95% N2/5% CO2 gas through the system at a rate of 20-25 Lpm for at least 5 minutes. The system was incubated in the incubator at 37° C. for 4 hours, with degassing of the chamber once more after 1 hour.

At the conclusion of the insult procedure, OGD medium was aspirated and warm medium was added to the neurons. 24-28 hours later, PDAC and neurons were plated at equal numbers at 100,000 cells each per well of a 6-well plate suspended in Neuronal Medium were added to the neurons and co-cultured for 6 days.

Photomicrographs were taken of random fields in a 6-well plate for each condition. Cells having a typical neuron morphology were identified, and neurite lengths were recorded. The average length of the neurites positively correlated to neuronal health, and were longer in co-cultures of neurons and PDAC, indicating that the PDAC were protecting the cells from the insult.

### Reactive Oxygen Species Assay

PDAC were determined to express superoxide dismutase, catalase, and heme oxygenase gene during hypoxia. The ability of PDAC to scavenge reactive oxygen species, and to protect cells from such species, was determined in an assay using hydrogen peroxide as a reactive oxygen species generator.

Assay Description: Target cells (Astrocytes, ScienCell Research Laboratories) were seeded in 96-well black well plates pre-coated with poly-L-lysine at 6000/cm2. The astrocytes are allowed to attach overnight in growth medium at 37° C. with 5% carbon dioxide. The following day, the culture media was removed and the cells were incubated with cell permeable dye DCFH-DA (Dichlorofluorescin diacetate), which is a fluorogenic probe. Excess dye was removed by washing with Dulbecco's Phosphate Buffered Saline or Hank's Buffered Salt Solution. The cells were then insulted with reactive oxygen species by addition of 1000 μM hydrogen peroxide for 30-60 minutes. The hydrogen peroxide-containing medium was then removed, and replaced with serum-free, glucose-free growth medium. PDAC were added at 6000/cm2, and the cells were cultured for another 24 hours. The cells were then read in a standard fluorescence plate reader at 480Ex and 530Em. The reactive oxygen species content of the medium was directly proportional to the levels of DCFH-DA in the cell cytosol. The reactive oxygen species content was measured by comparison to pre-determined DCF standard curve. All experiments were done with N=24.

For the assay, 1×DCFH-DA was prepared immediately prior to use by diluting a 20×DCFH-DA stock solution to 1× in cell culture media without fetal bovine serum, and stirring to homogeneity. Hydrogen Peroxide (H202) dilutions were prepared in DMEM or DPBS as necessary. A standard curve was prepared as a 1:10 dilution series in concentration range 0 μM to 10 μM by diluting 1 mM DCF standard in cell culture media, transferring 100 μl of DCF standard to a 96 well plate suitable for fluorescent measurement, and adding 100 μl of cell lyses buffer. Fluorescence was read at 480Ex and 530Em.

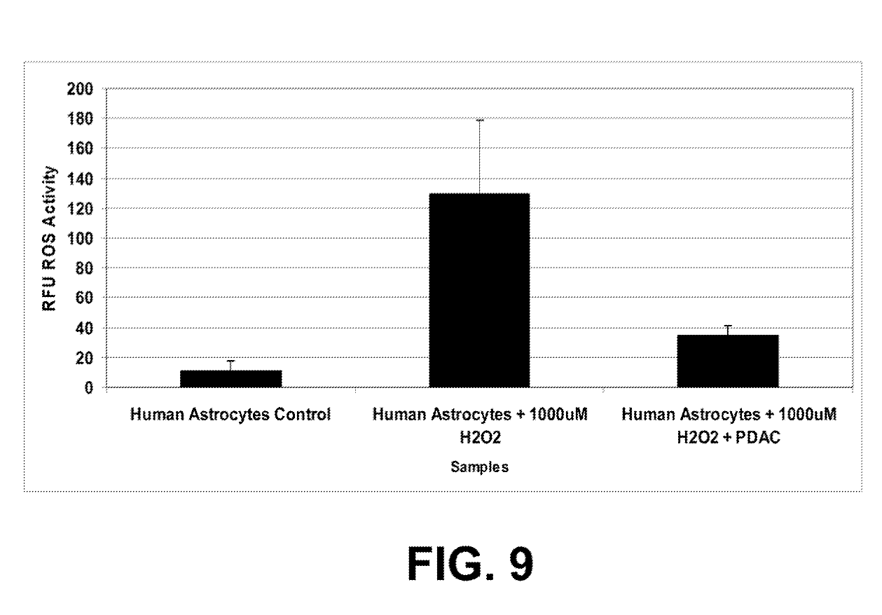

Results: PDAC significantly reduced the concentration of reactive oxygen species in the astrocyte co-cultures. See FIG. 9.

### 6.5 Example 5: Method of Treatment

6.5.1 Treatment of Diabetic Foot Ulcer Using Placental Stem Cells

A 52 year old male with type I diabetes presents with an ulcer on his left foot. A diagnosis of diabetic foot ulcer is made. After diagnosis, the subject is treated with CD10+, CD34−, CD105+, CD200+ placental stem cells according to the following regimen: 1×10⁶ to 3×10⁷ CD10+, CD34−, CD105+, CD200+ placental stem cells are administered intramuscularly. The individual is monitored over the next 24 months for signs of improvement in any symptom of the DFU, particularly to determine whether the DFU has reduced in size or closed. Therapeutic effectiveness is established if any of the symptoms of the DFU improve during the monitoring period.

### 6.6 Example 6: Dfu Treatment Protocol

Subjects having diabetic foot ulcer (DFU) with peripheral arterial disease (PAD), aged 18-80, are treated with CD10⁺, CD34⁻, CD105⁺, CD200⁺ placental stem cells. The placental stem cells are administered intramuscularly on days 1 (the first day of treatment) and 8 at the following doses: (i): 3×10⁶ CD10⁺, CD34⁻, CD105⁺, CD200⁺ placental stem cells; (ii): 1×10⁷ CD10⁺, CD34⁻, CD105⁺, CD200⁺ placental stem cells; or (iii) 3×10⁷ CD10⁺, CD34⁻, CD105⁺, CD200⁺ placental stem cells.

Clinical Endpoints

A primary clinical endpoint for efficacy of CD10⁺, CD34⁻, CD105⁺, CD200⁺ placental stem cells for treating DFU can be closure of the DFU or DFUs being treated. Ulcer closure can be represented by skin closure without drainage or need for dressing. Complete closure can be represented by retention of ulcer closure for at least four weeks following determination of closure. Ulcer closure can be assessed at three months following treatment with the placental stem cells.

Other clinical endpoints for efficacy of CD10+, CD34−, CD105+, CD200+ placental stem cells for treating DFU can include: (i) reduction of the frequency and severity of adverse events, which can be assessed up to 24-months following treatment; (ii) time to ulcer closure, which can be assessed at six months following treatment; (ii) improvement in ankle brachial index (ABI), which can be assessed at six months following treatment; (iii) improvement in toe brachial index (TBI), which can be assessed at six months following treatment; (iv) reduction in the size and number of DFUs, which can be assessed up to 24-months following treatment; (v) improvement in transcutaneous oxygen level, which can be assessed at six months following treatment; (vi) improvement in pulse volume recording, which can be assessed at six months following treatment; (vii) time to major amputation, which can be assessed up to 24-months following treatment; (viii) improvement on the Wagner Grading Scale, which can be assessed up to 24-months following treatment; (ix) improvement in Rutherford criteria, which can be assessed at six months following treatment; and (x) improvement in leg rest pain score, which can be assessed up to 24-months following treatment; and (xi) improvement in quality of life of the subject as assessed by (i) a 36-item Short Form Health Survey (SF-36) (see, e.g., Ware et al., Medical Care 30(6):473-483); (ii) the Diabetic Foot Ulcer Scale Short Form (DFS-SF) (see, e.g., Bann et al., Pharmacoeconomics, 2003, 21(17):1277-90); (iii) the Patient Global Impression of Change Scale (see, e.g., Kamper et al., J. Man. Manip. Ther., 2009, 17(3):163-170); and/or (iv) the EuroQol5D (EQ-5D™) Scale.

Subject Selection

The following eligibility criteria may be used to select subjects for whom treatment with CD10+, CD34−, CD105+, CD200+ placental stem cells is considered appropriate. All relevant medical and non-medical conditions are taken into consideration when deciding whether this treatment protocol is suitable for a particular subject.

Subjects should meet the following conditions to be eligible for the treatment protocol:
Males and females, at least 18 years of age or older.
Understand and voluntarily sign an informed consent document prior to any study related assessments/procedures are conducted.
Able to adhere to the study visit schedule and other protocol requirements.
Diabetes mellitus Type 1 or Type 2.
Diabetic foot ulcer with severity of Grade 1 (full thickness only) or Grade 2 on the Wagner Grading Scale of greater than one month duration which has not adequately responded to conventional ulcer therapy with a size of at least of 1 cm² except if present on the toe. The maximum lesion size range in the index ulcer is ≦6.25 cm². The measurement of the index ulcer is to be evaluated and measured after debridement (if necessary) at the Screening Visit.
Subjects that meet one or more of the following criteria of arterial insufficiency in the foot with the index ulcer:
   a. Peripheral arterial disease with ABI≧0.4 and ≦0.8 or TBI>0.30 and ≦0.65.
   b. Transcutaneous oxygen (TcPO2) measurement between 20-40 mmHg. The area measured with TcPO2 should be free of edema and thickened skin.
No planned revascularization or amputation over the next 3 months after screening visit.
Screening should not begin until at least 14 days after a failed reperfusion intervention and at least 30 days after a successful reperfusion intervention.
Subjects should be receiving appropriate medical therapy for hypertension and diabetes and any other chronic medical conditions for which they require ongoing care.
A female of childbearing potential (FCBP) must have a negative serum pregnancy test at Screening and a negative urine pregnancy test prior to treatment with study therapy. In addition, sexually active FCBP must agree to use 2 of the following adequate forms of contraception methods simultaneously such as: oral, injectable, or implantable hormonal contraception; tubal ligation; IUD; barrier contraceptive with spermicide or vasectomized partner for the duration of the study and the Follow-up Period.
Males (including those who have had a vasectomy) must agree to use barrier contraception (latex condoms) when engaging contraception (latex condoms) in reproductive sexual activity with FCBP for the duration of the study and the Follow-up Period

Subjects having one or more of the following conditions can be excluded from the treatment protocol:
Any significant medical condition, laboratory abnormality, or psychiatric illness that would prevent the subject from participating in the study.
Any condition including the presence of laboratory abnormalities, which places the subject at unacceptable risk if he or she were to participate in the study.
Any condition that confounds the ability to interpret data from the study.
Known to be positive for human immunodeficiency virus, Hepatitis C virus, or active infection with Hepatitis B virus.
Pregnant or lactating females.
Subjects with a body mass index >40 at Screening.
AST (SGOT) or ALT (SGPT)>2.5× the upper limit of normal (ULN) at Screening.
Estimated glomerular filtration rate (eGFR)<30 mL/min/1.73 m² at Screening calculated using the Modification of Diet in Renal Disease Study equation (Levey, 2006) or history of eGFR decline >15 mL/min/1.73 m² in the past year.
Alkaline phosphatase >2.5× the ULN at Screening.
Bilirubin level >2 mg/dL (unless subject has known Gilbert's disease) at Screening.
Untreated chronic infection or treatment of any infection with systemic antibiotics, including the ulcer site, must be free of antibiotics within 1 week prior to dosing with IP.
Active osteomyelitis, infection, or cellulitis at or adjacent to the index ulcer.
Index ulcer that has decreased or increased in size by ≧30% during the Screening/Run-In Period.
Pain at rest due to limb ischemia.
Transcutaneous oxygen measurements ≦20 mmHg in the foot with the index ulcer.
Heel ulcers.
Uncontrolled hypertension (defined as diastolic blood pressure >100 mmHg or systolic blood pressure >180 mmHg during Screening at 2 independent measurements taken while subject is sitting and resting for at least 5 minutes).
Poorly controlled diabetes mellitus (hemoglobin A1c>12% or a screening serum glucose of 300 mg/dl).
Untreated proliferative retinopathy.
History of malignant ventricular arrhythmia, CCS Class III-IV angina pectoris, myocardial infarction/percutaneous coronary intervention (PCI)/coronary artery bypass graft (CABG) in the preceding 6 months prior to signing the informed consent form (ICF), pending coronary revascularization in the following 3 months, transient ischemic attack/cerebrovascular accident in the preceding 6 months, prior to signing the ICF, and/or New York Heart Association [NYHA] Stage III or IV congestive heart failure.
Abnormal ECG: new right bundle branch block (BBB)≧120 msec in the preceding 3 months prior to signing the ICF.
Uncontrolled hypercoagulation.
Life expectancy less than 2 years at the time of signing the ICF due to concomitant illnesses.
In the opinion of the Investigator, the subject is unsuitable for cellular therapy.
History of malignancy within 5 years prior to signing the ICF except basal cell or squamous cell carcinoma of the skin or remote history of cancer now considered cured or positive Pap smear with subsequent negative follow-up.
History of hypersensitivity to any of the components of the product formulation (including bovine or porcine products, dextran 40, and dimethyl sulfoxide [DMSO]).
Subject has received an investigational agent—an agent or device not approved by the US Food and Drug Administration (FDA) for marketed use in any indication—within 90 days (or 5 half-lives, whichever is longer) prior to treatment with study therapy or planned participation in another therapeutic study prior to the completion of this study.
Subject has received previous investigational gene or cell therapy.

Clinical Outcome

Efficacy of the CD10⁺, CD34⁻, CD105⁺, CD200⁺ placental stem cells in treatment of DFU is confirmed if improvement in one or more clinical endpoints is demonstrated.

### 6.7 Example 7: Alternate Dfu Treatment Protocol

Subjects having diabetic foot ulcer (DFU) with peripheral arterial disease (PAD), at least 18 years of age, are treated with CD10⁺, CD34⁻, CD105⁺, CD200⁺ placental stem cells. Subject Group I: 3×10⁶CD10⁺, CD34⁻, CD105⁺, CD200⁺ placental stem cells are administered intramuscularly on days 1 (the first day of treatment), 29, and 57. Subject Group II: 3×10⁷ CD10⁺, CD34⁻, CD105⁺, CD200⁺ placental stem cells are administered intramuscularly on days 1 (the first day of treatment), 29, and 57. Subject Group III: placebo is administered intramuscularly on days 1 (the first day of treatment), 29, and 57.

Clinical Endpoints

A primary clinical endpoint for efficacy of CD10⁺, CD34⁻, CD105⁺, CD200⁺ placental stem cells for treating DFU can be improvement in limb vascular function as assessed by measurement of ankle brachial index (ABI); transcutaneous oximetry (TCOM), near infrared spectroscopy, Fludeoxyglucose positron emission tomography/computed tomography (FGD PET/CT), Doppler ultrasound, magnetic resonance imaging (MRI), angiography, and/or oximetry. Improvement in limb vascular function can be assessed at approximately one year following treatment.

Other clinical endpoints for efficacy of CD10+, CD34−, CD105+, CD200+ placental stem cells for treating DFU can include: (i) ulcer closure and complete wound closure of the index ulcer (ulcer closure can be represented by skin closure without drainage or need for dressing; complete closure can be represented by retention of ulcer closure for at least four weeks following determination of closure), which can be assessed at approximately one year following treatment; (ii) reduction of the frequency and severity of adverse events, which can be assessed at approximately one year following treatment; (iii) reduction in the number, size of all ulcers and 50% closure of the index ulcer, which can be assessed at approximately one year following treatment; (iv) a reduction in time to major amputation of the treated leg, which can be assessed at approximately one year following treatment; (v) improvement on the Wagner Grading Scale, which can be assessed at approximately one year following treatment; (vi) improvement in Rutherford criteria, which can be assessed at approximately one year following treatment; (vii) improvement in leg rest pain score, which can be assessed at approximately one year following treatment; and (viii) improvement in quality of life of the subject as assessed using the Patient Global Impression of Change in Neuropathy (PGICN).

Subject Selection

The following eligibility criteria may be used to select subjects for whom treatment with CD10⁺, CD34⁻, CD105⁺, CD200⁺ placental stem cells is considered appropriate. All relevant medical and non-medical conditions are taken into consideration when deciding whether this treatment protocol is suitable for a particular subject.

Subjects should meet the following conditions to be eligible for the treatment protocol:
Males and females, at least 18 years of age or older.
Diabetes mellitus Type 1 or Type 2.
Diabetic foot ulcer with severity of Grade 1 (full thickness only) or Grade 2 on the Wagner Grading Scale of greater than one month duration which has not adequately responded to conventional ulcer therapy.
Subjects that meet one or more of the following criteria of arterial insufficiency in the foot with the index ulcer:
   a. Peripheral arterial disease with ABI≧0.4 and ≦0.8 or TBI≧0.30 and ≦0.65.
   b. Transcutaneous oxygen (TcPO2) measurement between 20-40 mmHg.
No planned revascularization or amputation over the next 3 months after screening visit.
Dosing should not begin until at least 14 days after a failed reperfusion intervention and at least 30 days after a successful reperfusion intervention.

Subjects having one or more of the following conditions can be excluded from the treatment protocol:
Any significant medical condition, laboratory abnormality, or psychiatric illness that would prevent the subject from participating in the study.
Any condition including the presence of laboratory abnormalities, which places the subject at unacceptable risk if he or she were to participate in the study.
Pregnant or lactating females.
Subjects with a body mass index >40 at Screening.
Estimated glomerular filtration rate (eGFR)<30 mL/min/1.73 m² at Screening calculated using the Modification of Diet in Renal Disease Study equation (Levey, 2006) or history of eGFR decline >15 mL/min/1.73 m² in the past year.
Untreated chronic infection or treatment of any infection with systemic antibiotics, including the ulcer site, must be free of antibiotics within 1 week prior to dosing with IP.
Known osteomyelitis, infection, or cellulitis at or adjacent to the index ulcer.
Limb pain at rest due to limb ischemia.
Uncontrolled hypertension (defined as diastolic blood pressure >100 mmHg or systolic blood pressure >180 mmHg during Screening at 2 independent measurements taken while subject is sitting and resting for at least 5 minutes).
Poorly controlled diabetes mellitus (hemoglobin A1c>12% or a screening serum glucose of ≧300 mg/dl).
Untreated proliferative retinopathy.
History of malignant ventricular arrhythmia, CCS Class III-IV angina pectoris, myocardial infarction/percutaneous coronary intervention (PCI)/coronary artery bypass graft (CABG) in the preceding 6 months prior to signing the informed consent form (ICF), pending coronary revascularization in the following 3 months, transient ischemic attack/cerebrovascular accident in the preceding 6 months, prior to signing the ICF, and/or New York Heart Association [NYHA] Stage III or IV congestive heart failure.
Abnormal ECG: new right bundle branch block (BBB)≧120 msec in the preceding 3 months prior to signing the ICF.
Uncontrolled hypercoagulation.
Life expectancy less than 2 years at the time of signing the ICF due to concomitant illnesses.
In the opinion of the Investigator, the subject is unsuitable for cellular therapy.
History of malignancy within 5 years prior to signing the ICF except basal cell or squamous cell carcinoma of the skin or remote history of cancer now considered cured or positive Pap smear with subsequent negative follow-up.
History of hypersensitivity to any of the components of the product formulation (including bovine or porcine products, dextran 40, and dimethyl sulfoxide [DMSO]).
Subject has received an investigational agent—an agent or device not approved by the US Food and Drug Administration (FDA) for marketed use in any indication—within 90 days (or 5 half-lives, whichever is longer) prior to treatment with study therapy or planned participation in another therapeutic study prior to the completion of this study.
Subject has received previous investigational gene or cell therapy.

Clinical Outcome

Efficacy of the CD10⁺, CD34⁻, CD105⁺, CD200⁺ placental stem cells in treatment of DFU is confirmed if improvement in one or more clinical endpoints is demonstrated.

### 6.8 Example 8: Results of Dfu Treatment Protocol

Subjects with diabetic foot ulcer (DFU) were treated with CD10⁺, CD34⁻, CD105⁺, CD200⁺ placental stem cells in a Phase I clinical study, similar to the one outlined in Example 6, above.

Fifteen subjects were enrolled. Placental stem cells were administered according to the following regimen: (i) 3×10⁶ CD10+, CD34−, CD105+, CD200+ placental stem cells were administered intramuscularly to 3 subjects; (ii) 1×10⁷ CD10+, CD34−, CD105+, CD200+ placental stem cells were administered intramuscularly to 3 subjects; (iii) 3×10⁷ CD10+, CD34−, CD105+, CD200+ placental stem cells were administered intramuscularly to 3 subjects; and (iv) 1×10⁸ CD10+, CD34−, CD105+, CD200+ placental stem cells were administered intramuscularly to 6 subjects.

No treatment-related serious adverse events (SAE) or treatment-related deaths were recorded.

Seven of the treated subjects demonstrated positive results within three months of administration of placental stem cells. Five subjects demonstrated ulcer closure; two subjects demonstrated partial (˜50%) ulcer healing. A trend toward an increase in ankle brachial index (ABI) was observed among the treated patients. Additionally, an increase in ABI was observed in patients whose DFU healed as compared with patients whose DFU did not heal.

The results indicate that intramuscular administration of CD10⁺, CD34⁻, CD105⁺, CD200⁺ placental stem cells to human subjects having diabetic foot ulcer was safe and well-tolerated. Further, the results indicate that treatment of human subjects having diabetic foot ulcer with CD10⁺, CD34⁻, CD105⁺, CD200⁺ placental stem cells can result in improvement in symptoms of the diabetic foot ulcer, as well as in closure of the diabetic foot ulcer altogether.

### 6.9 Example 9: Placental Stem Cells Promote Wound Healing in an Animal Model of Peripheral Artery Disease

6.9.1 PDACs Promote Blood Flow and Angiogram Score in Ischemic Mice

To assess the efficacy of CD10⁺, CD34⁻, CD105⁺, CD200⁺ placental stem cells on vascular regeneration and wound healing, a surgical mouse model of diabetic limb ischemia was used. To model the effects of diabetic limb ischemia, db/db diabetic mice were subjected to hindlimb ischemia (HLI) surgery and monitored for various levels of wound repair, inflammation, and revascularization with and without subsequent administration CD10⁺, CD34⁻, CD105⁺, CD200⁺ placental stem cells.

Hindlimb ischemia (HLI) surgery was performed as described. See, e.g., Goto et al., Tokai J. Exp. Clin. Med. 31(3):128-132 (2006).

One day following HLI surgery, cryopreserved placental stem cells were thawed at 37° C. Within two hours post-thaw, either 50 μl placental stem cells or 50 μl vehicle control was administered intramuscularly near the site of the surgical wound. Placental stem cells were administered at dosages of 3,000 cells, 30,000 cells, or 300,000 cells and blood flow and angiogram score were determined at 1 day, 14 days, 28 days, 42 days, and 49 days post-surgery. Blood flow was measured using non-contact laser Doppler and was normalized to the blood flow of the corresponding non-surgical limb. Angiogram score was measured according to Bollinger et al., Atherosclerosis, 1981, 38(3-4):339-46.

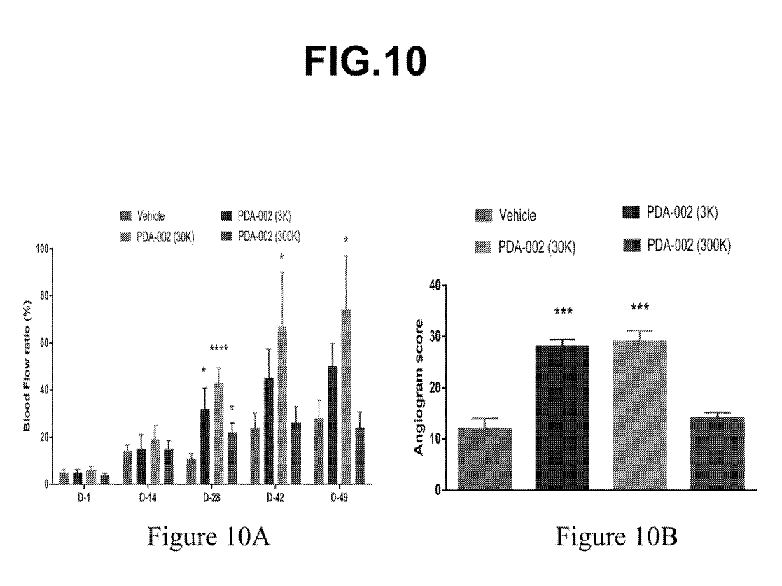

As shown in FIG. 10A, administration of CD10⁺, CD34⁻, CD105⁺, CD200⁺ placental stem cells resulted in an increase in blood flow following HLI beginning 28 days post-surgery and persisting through the end of the observation period. All dosages of administered placental stem cells were effective at day 28, and dosages of 30,000 cells per administration were effective at all time points measured as compared to vehicle control-treated animals. Similarly, as shown in FIG. 10B, administration of either 3,000 or 30,000 placental stem cells was effective at improving Angiogram score as compared to vehicle control treated animals. These data indicate that the administration of CD10⁺, CD34⁻, CD105⁺, CD200⁺ placental stem cells can improve clinical measures of ischemic wound healing in an animal model of peripheral artery disease and type 2 diabetes.

### 6.9.2 CD10⁺, CD34⁻, CD105⁺, CD200⁺ Placental Stem Cells Promote Capillary Density and Vessel Maturation in Ischemic Mice

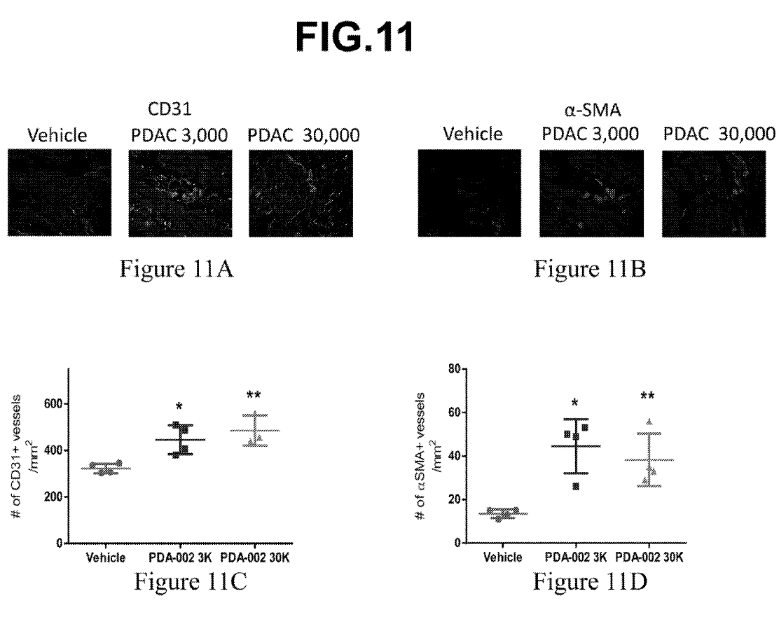

HLI surgery was performed followed by intramuscular administration of CD10⁺, CD34⁻, CD105⁺, CD200⁺ placental stem cells as in Section 6.9.1, supra. Following administration of either (i) vehicle control, (ii) 3,000 PDACs, or (iii) 30,000 PDACs, quadriceps muscles were removed and fixed in HOPE fixative and embedded in paraffin. Tissues were sectioned and stained according to standard procedures. Three sections from three representative animals in each treatment group were digitally imaged and quantified. As shown in FIG. 11, animals treated with either dosage of PDACs showed an increase in CD31 staining of blood vessels (FIGS. 11A and 11C) and an increase in α-smooth muscle actin of blood vessel walls (FIGS. 11B and 11D) as compared to vehicle control treated animals. These data indicate that the treatment of ischemic limbs with CD10⁺, CD34⁻, CD105⁺, CD200⁺ placental stem cells can promote the growth and maturation of new blood vessels following acute ischemic injury.

6.9.3 CD10⁺, CD34⁻, CD105⁺, CD200⁺ Placental Stem Cells Promote Muscle Repair and Reduce Adipose Infiltration in Ischemic Tissue

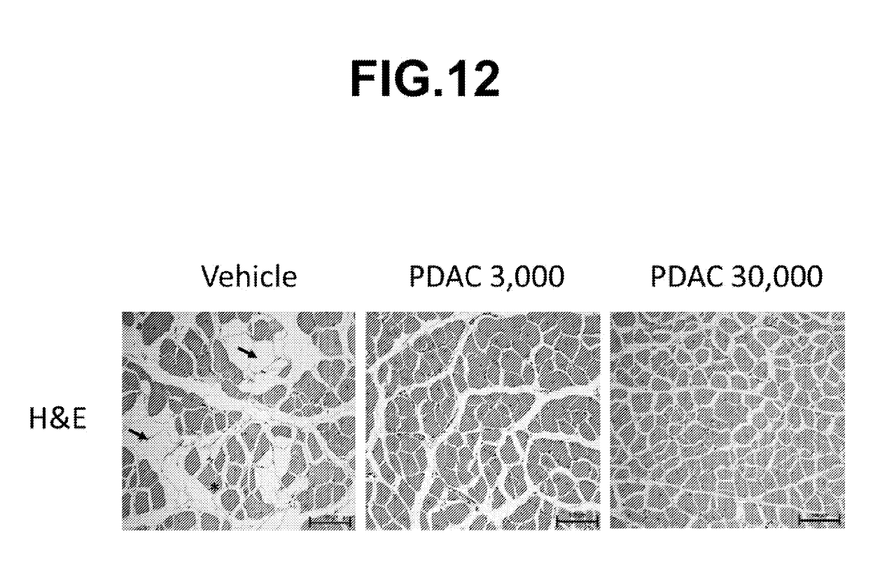

HLI surgery and treatment with CD10⁺, CD34⁻, CD105⁺, CD200⁺ placental stem cells followed by quadriceps isolation and sectioning were performed as in Sections 6.9.1 and 6.9.2, supra. Sectioned tissues were stained with H&E according to standard procedures. As shown in FIG. 12, placental stem cell treated ischemic animals had a higher number of myofibers with central nuclei and less infiltration of adipose tissue (see arrow) as compared to vehicle control treated animals. Nucleated myofibers are associated with muscular regeneration, while adipose tissue infiltration of muscle tissue is associated with pro-inflammatory immune responses thought to contribute to muscle weakening and degeneration (Donath & Shoelson, Nat Rev Immunol. 2011 February; 11(2):98-107). Together, these data suggest that CD10⁺, CD34⁻, CD105⁺, CD200⁺ placental stem cells promote muscle regeneration in ischemic animals and may prevent inflammatory signaling following acute ischemic injury.

6.9.4 CD10⁺, CD34⁻, CD105⁺, CD200⁺ Placental Stem Cells Administration Shifts Macrophage Differentiation Towards M2 Phenotype in Adipose Tissue Following Hindlimb Ischemia

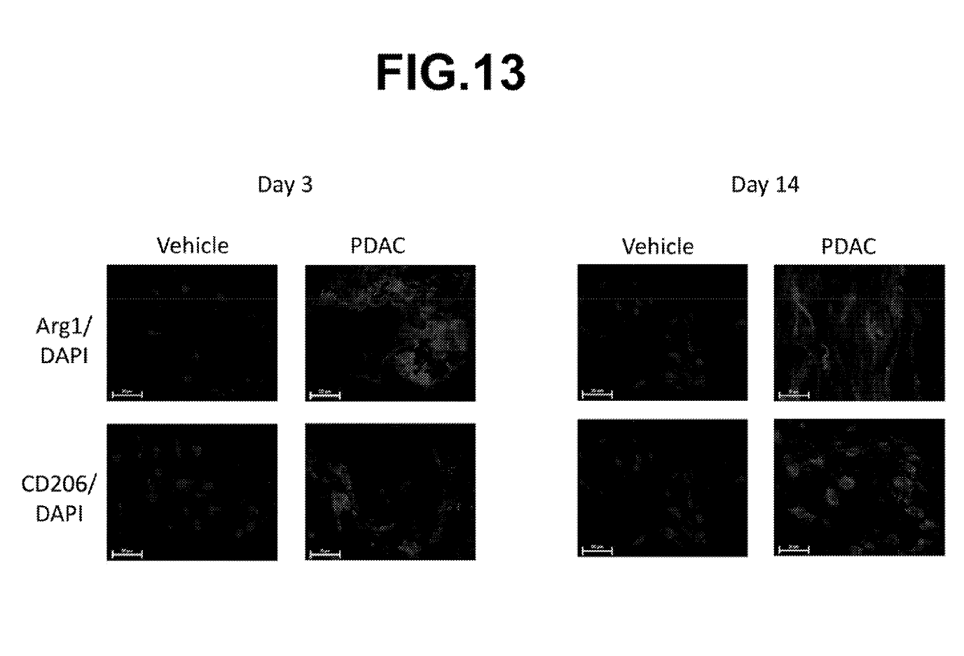

HLI surgery and treatment with CD10⁺, CD34⁻, CD105⁺, CD200⁺ placental stem cells was performed as in Section 6.9.1, supra. Inguinal fat pads from 3 days and 14 days post-surgery were isolated and stained with DAPI and antibodies to either Arg1 or CD206 (markers of M2 macrophages) to determine macrophage phenotype. As shown in FIG. 13, PDAC-treated animals exhibited higher levels of anti-inflammatory M2 macrophages at both 3 days and 14 days post-surgery as compared to vehicle control treated animals. These data show that M2 macrophages are present a much higher level in CD10⁺, CD34⁻, CD105⁺, CD200⁺ placental stem cell treated animals as compared to vehicle control treated animals following ischemic injury.

6.9.5 CD10⁺, CD34⁻, CD105⁺, CD200⁺ Placental Stem Cells Administration Modulates Adipokine Production in Adipose Tissue

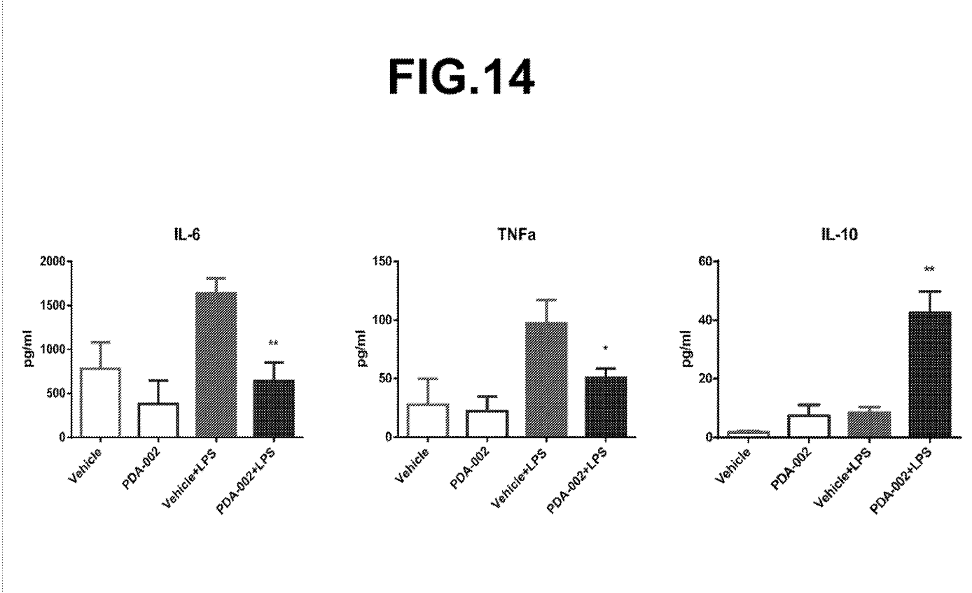

Following HLI surgery and treatment with CD10⁺, CD34⁻, CD105⁺, CD200⁺ placental stem cells followed by inguinal fat pad isolation as in Section 6.9.4, supra, inguinal fat pads were dissociated into single cell suspension and analyzed for the secretion of several cytokines. Briefly, 1×10¹⁵ adipose cells from placental stem cell treated and vehicle control treated animals were plated in a 96-well plate, and half of the wells were stimulated with 1 μg/ml lipopolysaccharide (LPS) for 24 hours. Supernatants from stimulated and unstimulated cells were then isolated and cytokine levels were determined using the MAP Cytokine/Chemokine Magnetic Bead Panel (Millipore). As shown in FIG. 14, the pro-inflammatory cytokines IL-6 and TNFα were reduced in placental stem cell treated cell suspensions after LPS stimulation as compared to vehicle control treated animals, while the anti-inflammatory cytokine IL-10 was increased in placental stem cell treated cells suspensions after LPS stimulation as compared to vehicle control treated animals. These results indicate that CD10⁺, CD34⁻, CD105⁺, CD200⁺ placental stem cells can modulate the inflammatory status in adipose cells by both inhibiting pro-inflammatory cytokines and simultaneously promoting anti-inflammatory cytokines.

### 6.10 Example 10: DFU Treatment Protocol

Subjects having diabetic foot ulcer (DFU) with or without peripheral arterial disease (PAD), aged 18-80, are treated with CD10⁺, CD34⁻, CD105⁺, CD200⁺ placental stem cells. The placental stem cells are administered intramuscularly on days 1 (the first day of treatment) and 8 at the following doses: (i): 3×10⁶ CD10⁺, CD34⁻, CD105⁺, CD200⁺ placental stem cells; (ii): 1×10⁷ CD10⁺, CD34⁻, CD105⁺, CD200⁺ placental stem cells; or (iii) 3×10⁷ CD10⁺, CD34⁻, CD105⁺, CD200⁺ placental stem cells.

Clinical Endpoints

A primary clinical endpoint for efficacy of CD10⁺, CD34⁻, CD105⁺, CD200⁺ placental stem cells for treating DFU can be closure of the DFU or DFUs being treated. Ulcer closure can be represented by skin closure without drainage or need for dressing. Complete closure can be represented by retention of ulcer closure for at least four weeks following determination of closure. Ulcer closure can be assessed at three months following treatment with the placental stem cells.

Other clinical endpoints for efficacy of CD10+, CD34−, CD105+, CD200+ placental stem cells for treating DFU can include: (i) reduction of the frequency and severity of adverse events, which can be assessed up to 24-months following treatment; (ii) time to ulcer closure, which can be assessed at six months following treatment; (ii) improvement in ankle brachial index (ABI), which can be assessed at six months following treatment; (iii) improvement in toe brachial index (TBI), which can be assessed at six months following treatment; (iv) reduction in the size and number of DFUs, which can be assessed up to 24-months following treatment; (v) improvement in transcutaneous oxygen level, which can be assessed at six months following treatment; (vi) time to major amputation, which can be assessed up to 24-months following treatment; (vii) improvement on the Wagner Grading Scale, which can be assessed up to 24-months following treatment; (viii) improvement in Rutherford criteria, which can be assessed at six months following treatment; (ix) improvement in leg rest pain score, which can be assessed up to 24-months following treatment; and (x) improvement in quality of life of the subject as assessed by (a) a 36-item Short Form Health Survey (SF-36) (see, e.g., Ware et al., Medical Care 30(6):473-483); (b) the Diabetic Foot Ulcer Scale Short Form (DFS-SF) (see, e.g., Bann et al., Pharmacoeconomics, 2003, 21(17):1277-90); and/or (c) the Patient Global Impression of Change Scale (see, e.g., Kamper et al., J. Man. Manip. Ther., 2009, 17(3):163-170); and/or (iv) the EuroQol5D (EQ-5D™) Scale.

Subject Selection

The following eligibility criteria may be used to select subjects for whom treatment with CD10+, CD34−, CD105+, CD200+ placental stem cells is considered appropriate. All relevant medical and non-medical conditions are taken into consideration when deciding whether this treatment protocol is suitable for a particular subject.

Subjects should meet the following conditions to be eligible for the treatment protocol:
Males and females, at least 18 years of age or older.
Understand and voluntarily sign an informed consent document prior to any study related assessments/procedures are conducted.
Able to adhere to the study visit schedule and other protocol requirements.
Diabetes mellitus Type 1 or Type 2.
Diabetic foot ulcer with severity of Grade 1 (full thickness only) or Grade 2 on the Wagner Grading Scale of greater than one month duration which has not adequately responded to conventional ulcer therapy with a size of at least of 1 cm² except if present on the toe. The maximum lesion size range in the index ulcer is ≦10 cm². The measurement of the index ulcer is to be evaluated and measured after debridement (if necessary) at the Screening Visit.
No planned revascularization or amputation over the next 3 months after screening visit.
Screening should not begin until at least 14 days after a failed reperfusion intervention and at least 30 days after a successful reperfusion intervention.
Subjects should be receiving appropriate medical therapy for hypertension and diabetes and any other chronic medical conditions for which they require ongoing care.
A female of childbearing potential (FCBP) must have a negative serum pregnancy test at Screening and a negative urine pregnancy test prior to treatment with study therapy. In addition, sexually active FCBP must agree to use 2 of the following adequate forms of contraception methods simultaneously such as: oral, injectable, or implantable hormonal contraception; tubal ligation; IUD; barrier contraceptive with spermicide or vasectomized partner for the duration of the study and the Follow-up Period.
Males (including those who have had a vasectomy) must agree to use barrier contraception (latex condoms) when engaging contraception (latex condoms) in reproductive sexual activity with FCBP for the duration of the study and the Follow-up Period

Subjects having one or more of the following conditions can be excluded from the treatment protocol:
Any significant medical condition, laboratory abnormality, or psychiatric illness that would prevent the subject from participating in the study.
Any condition including the presence of laboratory abnormalities, which places the subject at unacceptable risk if he or she were to participate in the study.
Any condition that confounds the ability to interpret data from the study.
Known to be positive for human immunodeficiency virus, Hepatitis C virus, or active infection with Hepatitis B virus.
Pregnant or lactating females.
Subjects with a body mass index >45 at Screening.
AST (SGOT) or ALT (SGPT)>2.5× the upper limit of normal (ULN) at Screening.
On renal dialysis for abnormal kidney function.
An ABI<0.4 and or TBI<0.3 in the leg with the index ulcer.
Alkaline phosphatase >2.5× the ULN at Screening.
Bilirubin level >2 mg/dL (unless subject has known Gilbert's disease) at Screening.
Untreated chronic infection or treatment of any infection with systemic antibiotics, including the ulcer site, must be free of antibiotics within 1 week prior to dosing with IP.
Active osteomyelitis, infection, or cellulitis at or adjacent to the index ulcer.
Index ulcer that has decreased or increased in size by ≧30% during the Screening/Run-In Period.
Pain at rest due to limb ischemia.
Transcutaneous oxygen measurements ≦20 mmHg in the foot with the index ulcer.
Heel ulcers.
Uncontrolled hypertension (defined as diastolic blood pressure >100 mmHg or systolic blood pressure >180 mmHg during Screening at 2 independent measurements taken while subject is sitting and resting for at least 5 minutes).
Poorly controlled diabetes mellitus (hemoglobin A1c>12% or a screening serum glucose of ≧300 mg/dl).
Untreated proliferative retinopathy.
History of malignant ventricular arrhythmia, CCS Class III-IV angina pectoris, myocardial infarction/percutaneous coronary intervention (PCI)/coronary artery bypass graft (CABG) in the preceding 6 months prior to signing the informed consent form (ICF), pending coronary revascularization in the following 3 months, transient ischemic attack/cerebrovascular accident in the preceding 6 months, prior to signing the ICF, and/or New York Heart Association [NYHA] Stage III or IV congestive heart failure.
Abnormal ECG: new right bundle branch block (BBB)≧120 msec in the preceding 3 months prior to signing the ICF.
Uncontrolled hypercoagulation.
Life expectancy less than 2 years at the time of signing the ICF due to concomitant illnesses.
In the opinion of the Investigator, the subject is unsuitable for cellular therapy.
History of malignancy within 5 years prior to signing the ICF except basal cell or squamous cell carcinoma of the skin or remote history of cancer now considered cured or positive Pap smear with subsequent negative follow-up.
History of hypersensitivity to any of the components of the product formulation (including bovine or porcine products, dextran 40, and dimethyl sulfoxide [DMSO]).
Subject has received an investigational agent—an agent or device not approved by the US Food and Drug Administration (FDA) for marketed use in any indication—within 90 days (or 5 half-lives, whichever is longer) prior to treatment with study therapy or planned participation in another therapeutic study prior to the completion of this study.
Subject has received previous investigational gene or cell therapy.

Clinical Outcome

Efficacy of the CD10⁺, CD34⁻, CD105⁺, CD200⁺ placental stem cells in treatment of DFU is confirmed if improvement in one or more clinical endpoints is demonstrated.

### 6.11 Example 11: Circulating Endothelial Cells as a Biomarker for Treatment Efficacy

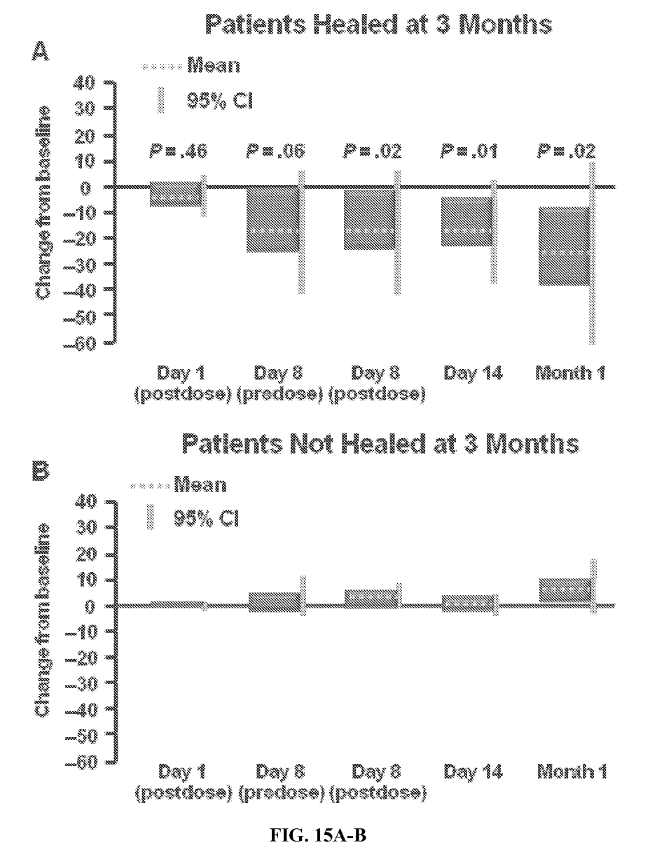

Numbers of circulating endothelial cells in subjects described in Example 8, above, were assessed at days 1, 8, 14, and 29 of the study period using the Veridex platform (Janssen Diagnostics). A statistically significant decrease in circulating endothelial cells was observed in subjects with healing DFU (n=5) throughout study day 8 to study day 29. See FIG. 15A; P values for change from baseline (Day 1, predose) were calculated using a nonparametric test of location of the median. Such a decrease was not observed in subjects with non-healing DFU. See FIG. 15B.

This example demonstrates that numbers of circulating endothelial cells can be used as biomarker to assess treatment efficicacy of DFU with CD10⁺, CD34⁻, CD105⁺, CD200⁺ placental stem cells.

### EQUIVALENTS

The present disclosure is not to be limited in scope by the specific embodiments described herein. Indeed, various modifications of the subject matter provided herein, in addition to those described, will become apparent to those skilled in the art from the foregoing description and accompanying figures. Such modifications are intended to fall within the scope of the appended claims.

Various publications, patents and patent applications are cited herein, the disclosures of which are incorporated by reference in their entireties.

What is claimed:

1. A method of treating a subject having a diabetic foot ulcer, comprising administering to the subject a composition comprising CD10⁺, CD34⁻, CD105⁺, CD200⁺ placental stem cells.

2. The method of claim 1, wherein said subject has more than one diabetic foot ulcers.

3. The method of claim 1 or 2, wherein said subject has peripheral arterial disease.

4. The method of any one of claims 1-3, wherein said composition comprising placental stem cells is administered intramuscularly.

5. The method of any one of claims 1-4, wherein said composition comprises between 1×10⁵ to 1×10⁶, 1×10⁶ to 3×10⁶, 3×10⁶ to 5×10⁶, 5×10⁶ to 1×10⁷, 1×10⁷ to 3×10⁷, 3×10⁷ to 5×10⁷, 5×10⁷ to 1×10⁸, 1×10⁸ to 3×10⁸, 3×10⁸ to 5×10⁸, 5×10⁸ to 1×10⁹, 1×10⁹ to 5×10⁹, or 5×10⁹ to 1×10¹⁰ placental stem cells.

6. The method of any one of claims 1-4, wherein said composition comprises about 1×10⁵, 3×10⁵, 5×10⁵, 1×10⁶, 3×10⁶, 5×10⁶, 1×10⁷, 3×10⁷, 5×10⁷, 1×10⁸, 3×10⁸, 5×10⁸, 1×10⁹, 5×10⁹, or 1×10¹⁰ placental stem cells.

7. The method of claim 6, wherein said composition comprises about 3×10⁶ placental stem cells.

8. The method of claim 6, wherein said composition comprises about 1×10⁷ placental stem cells.

9. The method of claim 6, wherein said composition comprises about 3×10⁷ placental stem cells.

10. The method of any one of claims 1-9, wherein said treatment results in a reduction in size of said diabetic foot ulcer.

11. The method of any one of claims 1-9, wherein said treatment results in closure of said diabetic foot ulcer.

12. The method of any one of claims 1-9, wherein said treatment results in improvement in one or more symptoms of said diabetic foot ulcer.

13. The method of claim 12, wherein said one or more symptoms is sores, ulcers, or blisters on the foot and/or lower leg of the subject; pain in the foot or feet and/or lower leg of the subject; difficulty walking; discoloration in the foot or feet of the subject; and/or infection of the foot or feet of the subject.

14. The method of any one of claims 1-9, wherein said treatment results in increased time to closure of the diabetic foot ulcer, improvement in ankle brachial index (ABI) of the subject, improvement in toe brachial index (TBI) of the subject, improvement in transcutaneous oxygen level of the subject, improvement in pulse volume recording of the subject, reduced time to major amputation, improvement on the Wagner Grading Scale, improvement in Rutherford criteria, and/or improvement in leg rest pain score of the subject.

15. A method for treating DFU in a subject in need of treatment, comprising (a) determining the number of endothelial cells circulating in the bloodstream of the subject; (b) administering a composition comprising CD10⁺, CD34⁻, CD105⁺, CD200⁺ placental stem cells to the subject; and (c) determining the number of endothelial cells circulating in the bloodstream of the subject following administration of the placental stem cells, wherein a decrease in the number of circulating endothelial cells following administration of placental stem cells as compared to the number of circulating endothelial cells before administration of placental stem cells indicates that treatment of DFU in said subject is effective.

16. A method for treating DFU in a subject in need of treatment, comprising: (a) administering a composition comprising CD10⁺, CD34⁻, CD105⁺, CD200⁺ placental stem cells to the subject; (b) determining the number of endothelial cells circulating in the bloodstream of the subject at a first time point following administration of the placental stem cells; and (c) determining the number of endothelial cells circulating in the bloodstream of the subject at a second time point following administration of the placental stem cells, wherein a decrease in the number of circulating endothelial cells measured at the second time point as compared to the number of circulating endothelial cells measured at the first time indicates that treatment of DFU in said subject is effective.

17. The method of claim 15 or 16, wherein the subject is administered a subsequent dose of a composition comprising CD10⁺, CD34⁻, CD105⁺, CD200⁺ placental stem cells if treatment of DFU in said subject is effective.

18. The method of any one of claims 15-17, wherein said composition comprises between 1×10⁵ to 1×10⁶, 1×10⁶ to 3×10⁶, 3×10⁶ to 5×10⁶, 5×10⁶ to 1×10⁷, 1×10⁷ to 3×10⁷, 3×10⁷ to 5×10⁷, 5×10⁷ to 1×10⁸, 1×10⁸ to 3×10⁸, 3×10⁸ to 5×10⁸, 5×10⁸ to 1×10⁹, 1×10⁹ to 5×10⁹, or 5×10⁹ to 1×10¹⁰ placental stem cells.

19. The method of any one of claims 15-17, wherein said composition comprises about 1×10⁵, 3×10⁵, 5×10⁵, 1×10⁶, 3×10⁶, 5×10⁶, 1×10⁷, 3×10⁷, 5×10⁷, 1×10⁸, 3×10⁸, 5×10⁸, 1×10⁹, 5×10⁹, or 1×10¹⁰ placental stem cells.
